(19) United States
(12) Patent Application Publication
(10) Pub. No.: US 2026/0216179 A1
(43) Pub. Date: Jul. 30, 2026

(54) 1,5-NAPHTHYRIDINE DERIVATIVES AS KRAS ONCOPROTEIN INHIBITORS

(71) Applicant: Beta Pharma, Inc., Wilmington, DE (US)

(72) Inventors: Don Zhang, Princeton, NJ (US); Jirong Peng, Mequon, WI (US); Michael John Costanzo, Bonney Lake, WA (US); Michael Alan Green, Easton, PA (US); Michael Nicholas Greco, Lansdale, PA (US); Stephen Bolgunas, Swedesboro, NJ (US)

(21) Appl. No.: 19/123,462

(22) PCT Filed: Oct. 25, 2023
(86) PCT No.: PCT/US2023/077701

§ 371 Date: Apr. 23, 2025

Related U.S. Application Data
(60) Provisional application No. 63/420,831, filed on Oct. 31, 2022.
(60) Provisional application No. 63/510,236, filed on Jun. 26, 2023.

(51) Int. Cl. A61K 31/4995; A61K 31/496; A61K 31/5377; A61K 45/06; A61P 35/00; C07D 471/04; C07D 519/00

(52) U.S. Cl. CPC A61K 31/4995; A61K 31/496; A61K 31/5377; A61K 45/06; A61P 35/00; C07D 471/04; C07D 519/00

(57) ABSTRACT

The present invention is directed to inhibitors of Kirsten rat sarcoma virus (KRAS) oncoproteins, and more particularly to certain 1,5-napthyridine derivatives of Formula I:

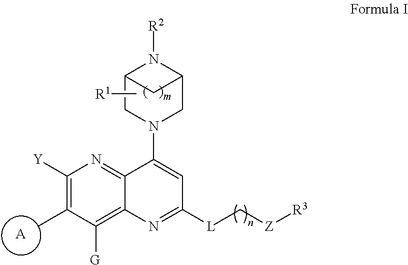

as well as compositions comprising Formula I and methods of using the compound of Formula I for the treatment or prevention of a disease, disorder, or medical condition mediated through KRAS, especially the KRAS G12C or KRAS G12D oncoproteins.

### CROSS-REFERENCE TO RELATED APPLICATIONS

This application claims the benefit of U.S. Provisional Application Ser. No. 63/420,831 filed Oct. 31, 2022, and U.S. Ser. No. 63/510,236 filed Jun. 26, 2023. Both of these applications are incorporated by reference herein in their entireties.

### BACKGROUND OF THE INVENTION

### Field of the Invention

The present invention is directed to inhibitors of Kirsten rat sarcoma virus (KRAS) oncoproteins, and more particularly to certain 1,5-napthyridine derivatives, compositions and methods for the treatment or prevention of a disease, disorder, or medical condition mediated through KRAS, especially the KRAS G12C and KRAS G12D oncoproteins. The diseases include various cancers.

### Brief Description of the Related Art

Ras is a superfamily of small guanosine triphosphate (GTP) binding proteins consisting of various isoforms. Ras genes can mutate to oncogenes that are associated with numerous cancers such as lung, pancreas, and colon. Ras is one of the most frequently mutated oncogenes. KRAS, (Kirsten rat sarcoma virus) an isoform of Ras, is one of the most frequently mutated Ras genes, comprising approximately 86% of all known mutations. KRAS functions as an On/Off switch in cell signaling. KRAS proteins are GTPases that operate between inactive (GDP-bound) and active (GTP-bound) states to control a variety of functions, including cell proliferation. However, mutated KRAS proteins lead to uncontrolled cell proliferation and cancer. The KRAS-4B proteoform is the major isoform in cancers of the colon (30-40%), lung (15-20%) and pancreas (90%) (Liu, P. et al., Acta Pharmaceutica Sinica B 2019, 9 (5), 871-879). Consequently, inhibitors of mutated KRAS proteins binding to GTP represent potential therapeutic agents for the treatment of various cancers.

Past attempts to design KRAS oncoprotein inhibitors have been mostly unsuccessful, due in large part to the high affinity of the KRAS oncoproteins for GTP. However, more recent approaches that target KRAS G12C have shown more promise. This mutation exists in roughly 50% of lung cancers and approximately 10-20% of all KRAS G12 mutations. The cysteine residue of the mutation is positioned within the active site such that the sulfhydryl functionality can form a covalent bond with a suitably functionalized bound ligand (Liu, Acta Pharmaceutica Sinica B 2019). This approach has identified irreversible, covalent inhibitors of KRAS G12C that are undergoing clinical study. The KRAS G12D mutation is present in approximately 4% of all non-small cell lung cancers, 13% of all colorectal cancers, 25% of pancreatic ductal adenocarcinomas, and 1.7% of small cell lung cancers (Cerami, E. and Sawyers, C. L. Cancer Discovery 2017, 7 (8), 818-831). Given the prominent role that both KRAS G12C and KRAS G12D play as drivers of many malignancies, a need for new KRAS G12C and KRAS G12D inhibitors with improved selectivity, safety, and efficacy profiles exists.

### SUMMARY OF THE INVENTION

In one aspect, the present invention is directed to a compound of Formula I:

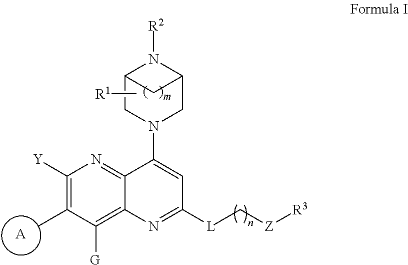

or a pharmaceutically acceptable salt, solvate, or prodrug thereof, wherein
A is chosen from aryl or heteroaryl optionally substituted with one or more of hydrogen, halogen, hydroxy, —C₁₋₆ alkyl, —C₂₋₆ alkenyl, —C₂₋₆ alkynyl, —C₀₋₃ alkyl(C₃₋₆ cycloalkyl),
C₁₋₆ alkyl(halo), —C₁₋₆ alkyl(OH), —O(C₁₋₄ alkyl), —C₁₋₃ alkyl(C₁₋₄ alkoxy), —CN, —CO₂R⁴, —CO₂N(R⁴)₂, —NO₂, —N(R⁴)₂, —P(O)(R⁵)₂, —SR₄, —S(O)R⁴, —SO₂R⁴ or a 5-6 membered heterocyclic ring;
   Y and G may be the same or different and chosen from hydrogen, halogen, C₁₋₄ alkyl, C₁₋₄ perdeuteroalkyl, —(C₀₋₂ alkyl)alkenyl, —(C₀₋₂ alkyl)alkynyl, —(C₀₋₂ alkyl)cycloalkyl, —C₁₋₄ haloalkyl, —O(C₁₋₄ alkyl), —S(C₁₋₄ alkyl), —(C₀₋₂ alkyl) cyano, —O(C₁₋₄ haloalkyl) or —S(C₁₋₄ haloalkyl);
   L is a bond, O, S, or NR⁴;
   m is 0-2;
   n is 0-2;
   Z is C (R⁴)₂ or a cyclic compound chosen from C₃₋₇ cycloalkyl, a saturated or partially unsaturated 4- to 7-membered nitrogen-containing ring, a saturated or partially unsaturated 7- to 10-membered nitrogen-containing bridged bicyclic ring;
   R¹ is chosen from hydrogen, hydroxy, halogen, —C₁₋₃ alkyl, —C₁₋₃ alkyl (OH), —C₁₋₃ alkyl(halo), —C₁₋₃ alkyl(C₁₋₃ alkoxy), —C₁₋₃ alkyl (CN) or —C₁₋₃ alkyl(P(O)R⁵₂);
   R² is chosen from hydrogen, —C(O)CH═CH, —C(O)CF═CH or —C(O)CCl═CH with the proviso that when R² is hydrogen, then m is either 1 or 2;
   R³ is chosen from hydrogen, halogen, hydroxy, —C₁₋₄ alkyl, —C₂₋₄ alkenyl, —C₂₋₄ alkynyl, —C₀₋₃ alkyl(C₃₋₆ cycloalkyl), —C₁₋₄ alkyl(halo), —C₁₋₄ alkyl (OH), —O(C₁₋₄ alkyl), —C₁₋₃ alkyl(C₁₋₃ alkoxy), —CN, —CO₂R⁴, —CO₂N(R⁴)₂, —NO₂, —N(R⁴)₂, —PO(R⁵)₂, —SR₄, —S(O)R⁴, —SO₂R⁴, or —(C₀₋₃ alkyl) R⁶;

R⁴ is chosen from is chosen from hydrogen, C₁₋₄ alkyl, aryl or heteroaryl;

R⁵ is chosen from hydrogen, hydroxy, C₁₋₄ alkyl, aryl, heteroaryl, C₁₋₄ alkoxy, aryloxy or heteroaryloxy;

R⁶ is chosen from N(R⁴)₂ or a 4- to 7-membered saturated or unsaturated heterocyclic ring containing one or more heteroatoms selected from the group N, O and S.

In another aspect, the present invention is directed to a pharmaceutical composition comprising the compound of Formula I, or a salt, solvate, or prodrug thereof, together with a pharmaceutically acceptable carrier.

In another aspect, the present invention is directed to a method of treating a disease, disorder, or medical condition in a patient, comprising the step of providing to a patient in need thereof a therapeutic agent, wherein the therapeutic agent is a compound of Formula I or a salt, solvate, or prodrug thereof.

### DETAILED DESCRIPTION OF THE INVENTION

### Terminology

Compounds are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced items. The term “or” means “and/or”. The terms “comprising,” “having,” “including,” and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include ¹¹C, ¹³C, and ¹⁴C. Accordingly, the compounds disclosed herein may include heavy or radioactive isotopes in the structure of the compounds or as substituents attached thereto. Examples of useful heavy or radioactive isotopes include ¹⁸F, ¹⁵N, ¹⁸O, ⁷⁶Br, ¹²⁵I and ¹³¹I.

All formulae disclosed herein include all salts of such Formulae.

The opened ended term “comprising” includes the intermediate and closed terms “consisting essentially of” and “consisting of.”

The term “substituted” means that any one or more hydrogens on the designated atom or group is replaced with a selection from the indicated group, provided that the designated atom's normal valence is not exceeded. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent.

A dash (“-”) that is not between two letters or symbols is used to indicate a point of attachment for a substituent.

“Alkyl” includes both branched and straight chain saturated aliphatic hydrocarbon groups, having the specified number of carbon atoms, generally from 1 to about 8 carbon atoms. The terms C₁₋₆ alkyl, C₁-C₆ alkyl and C₁-C₆ alkyl as used herein all indicate an alkyl group having from 1, 2, 3, 4, 5, or 6 carbon atoms. Other embodiments include alkyl groups having from 1 to 8 carbon atoms, 1 to 4 carbon atoms or 1 or 2 carbon atoms, e.g., C₁₋₈ alkyl, C₁₋₄ alkyl, and C₁₋₂ alkyl. When C₀₋ₙ alkyl is used herein in conjunction with another group, for example, —C₀₋₄ alkyl(phenyl), the indicated group, in this case phenyl, is either directly bound by a single covalent bond (C₀ alkyl), or attached by an alkyl chain having the specified number of carbon atoms, in this case 1, 2, 3, or 4 carbon atoms. Alkyls can also be attached via other groups such as heteroatoms as in —OC₀₋₄ alkyl(C₃₋₇ cycloalkyl). Examples of alkyl include, but are not limited to, methyl, ethyl, n-propyl, isopropyl, n-butyl, 3-methylbutyl, t-butyl, n-pentyl, and sec-pentyl.

“Alkoxy” is an alkyl group as defined above with the indicated number of carbon atoms covalently bound to the group it substitutes by an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or a “thioalkyl” group is an alkyl group as defined above with the indicated number of carbon atoms covalently bound to the group it substitutes by a sulfur bridge (—S—). Similarly, “alkenyloxy”, “alkynyloxy”, and “cycloalkyloxy” refer to alkenyl, alkynyl, and cycloalkyl groups, in each instance covalently bound to the group it substitutes by an oxygen bridge (—O—).

“Halo” or “halogen” means fluoro, chloro, bromo, or iodo, and are defined herein to include all isotopes of same, including heavy isotopes and radioactive isotopes. Examples of useful halo isotopes include ¹⁸F, ⁷⁶Br, and ¹³¹I. Additional isotopes will be readily appreciated by one of skill in the art.

“Haloalkyl” means both branched and straight-chain alkyl groups having the specified number of carbon atoms, substituted with 1 or more halogen atoms, generally up to the maximum allowable number of halogen atoms. Examples of haloalkyl include, but are not limited to, trifluoromethyl, difluoromethyl, 2-fluoroethyl, and penta-fluoroethyl.

“Haloalkoxy” is a haloalkyl group as defined above attached through an oxygen bridge (oxygen of an alcohol radical).

“Peptide” means a molecule which is a chain of amino acids linked together via amide bonds (also called peptide bonds).

“Pharmaceutical compositions” means compositions comprising at least one active agent, such as a compound or salt of Formula I, and at least one other substance, such as a carrier. Pharmaceutical compositions meet the U.S. FDA's GMP (good manufacturing practice) standards for human or non-human drugs.

“Carrier” means a diluent, excipient, or vehicle with which an active compound is administered. A “pharmaceutically acceptable carrier” means a substance, e.g., excipient, diluent, or vehicle, that is useful in preparing a pharmaceutical composition that is generally safe, non-toxic and neither biologically nor otherwise undesirable, and includes a carrier that is acceptable for veterinary use as well as human pharmaceutical use. A “pharmaceutically acceptable carrier” includes both one and more than one such carrier.

A “patient” means a human or non-human animal in need of medical treatment. Medical treatment can include treatment of an existing condition, such as a disease or disorder or diagnostic treatment. In some embodiments the patient is a human patient.

“Providing” means giving, administering, selling, distributing, transferring (for profit or not), manufacturing, compounding, or dispensing.

“Treatment” or “treating” means providing an active compound to a patient in an amount sufficient to measurably reduce any disease symptom, slow disease progression or cause disease regression. In certain embodiments treatment of the disease may be commenced before the patient presents symptoms of the disease.

A “therapeutically effective amount” of a pharmaceutical composition means an amount effective, when administered to a patient, to provide a therapeutic benefit such as an amelioration of symptoms, decrease disease progression, or cause disease regression.

A “therapeutic compound” means a compound which can be used for diagnosis or treatment of a disease. The compounds can be small molecules, peptides, proteins, or other kinds of molecules.

A significant change is any detectable change that is statistically significant in a standard parametric test of statistical significance such as Student's T-test, where p<0.05.

### Chemical Description

Compounds of the Formulae disclosed herein may contain one or more asymmetric elements such as stereogenic centers (e.g., asymmetric carbon atoms), stereogenic axes, rotamers with restricted rotation (e.g., atropisomers) and the like, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

All forms (for example solvates, optical isomers, enantiomeric forms, polymorphs, prodrugs, free base compound and salts) of the compounds of the invention may be employed either alone or in combination.

The term “chiral” refers to molecules, which have the property of non-superimposability of the mirror image partner.

“Stereoisomers” are compounds, which have identical chemical constitution, but differ with regard to the arrangement of the atoms or groups in space.

The term “solvate” refers to a chemical complex formed by the interaction of a solvent and a solute, such as the chemical compounds of the present invention.

The term “prodrug” refers to a biologically inactive compound which can be metabolized inside the body to produce a drug.

A “diastereomer” is a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis, crystallization in the presence of a resolving agent, or chromatography, using, for example a chiral HPLC column.

“Enantiomers” refer to two stereoisomers of a compound, which are non-superimposable mirror images of one another. A 50:50 mixture of enantiomers is referred to as a racemic mixture or a racemate, which may occur where there has been no stereoselection or stereospecificity in a chemical reaction or process.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory.

A “racemic mixture” or “racemate” is an equimolar (or 50:50) mixture of two enantiomeric species, devoid of optical activity. A racemic mixture may occur where there has been no stereoselection or stereospecificity in a chemical reaction or process.

A “chelating group” or “chelator” is a ligand group which can form two or more separate coordinate bonds to a single central atom, which is usually a metal ion. Chelating groups as disclosed herein are organic groups which possess multiple N, O, or S heteroatoms, and have a structure which allows two or more of the heteroatoms to form bonds to the same metal ion.

“Salts” include derivatives of the disclosed compounds in which the parent compound is modified by making inorganic and organic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media such as ether, ethyl acetate, ethanol, isopropanol, or acetonitrile are used, where practicable. Salts of the present compounds further include solvates of the compounds and of the compound salts. In an embodiment, the compounds of the present invention are synthesized or isolated as trifluoroacetic acid (TFA) salts.

In one embodiment, the salt forms of the compounds of the present invention described above may include pharmaceutically acceptable salts. Examples of pharmaceutically acceptable salts include, but are not limited to, non-toxic mineral or organic acid salts of basic residues such as amines; alkali or organic salts of acidic residues such as carboxylic acids; and the like. The pharmaceutically acceptable salts include the conventional salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in G. Steffen Paulekuhn, et al., Journal of Medicinal Chemistry 2007, 50, 6665 and Handbook of Pharmaceutically Acceptable Salts: Properties, Selection and Use, P. Heinrich Stahl and Camille G. Wermuth, Editors, Wiley-VCH, 2002.

In the preferred embodiments, the compounds of Formula I are represented by the structures 1a-1ah and 2a-2aw shown below, including pharmaceutically acceptable salts, solvates, or prodrugs thereof:

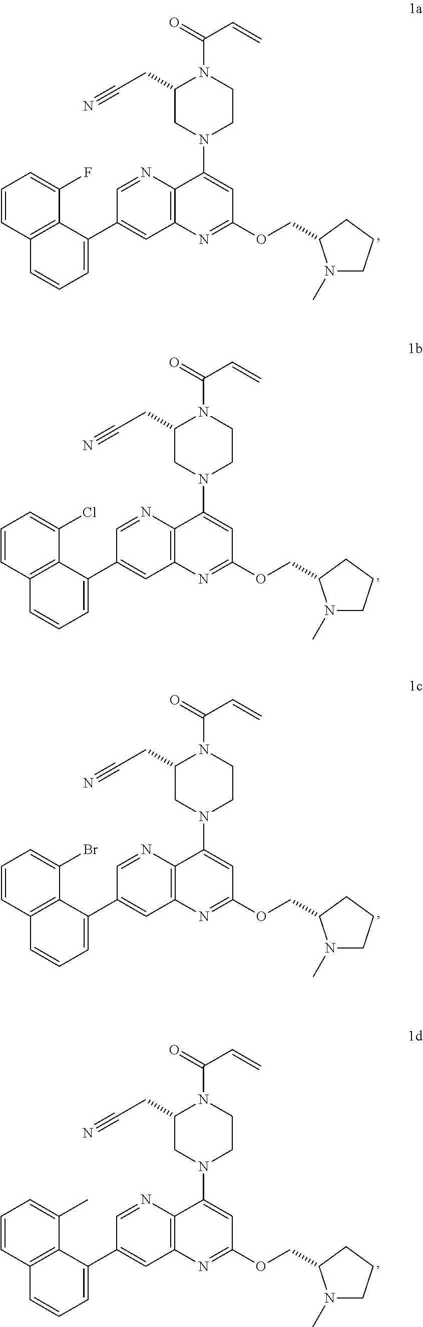

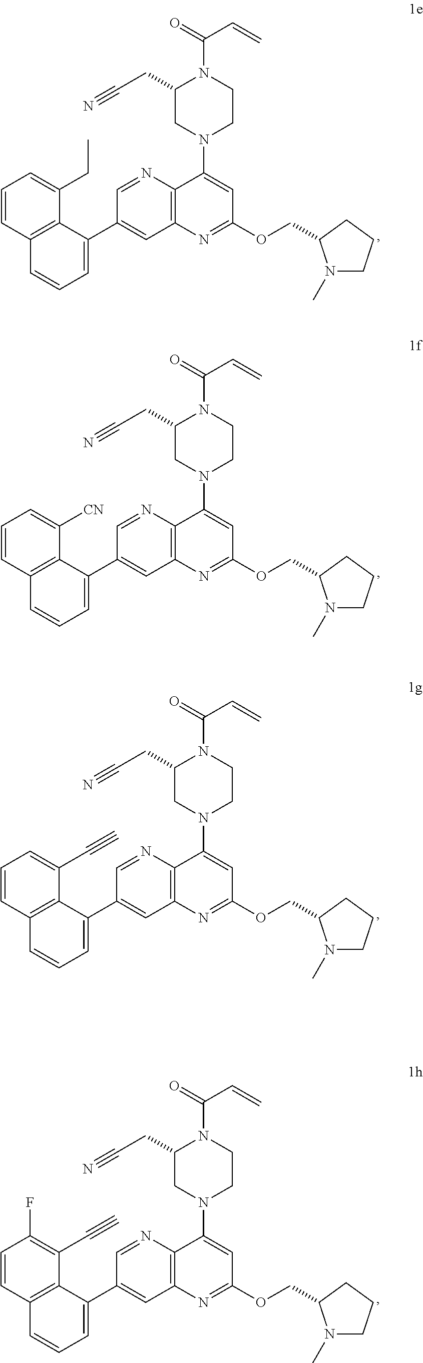

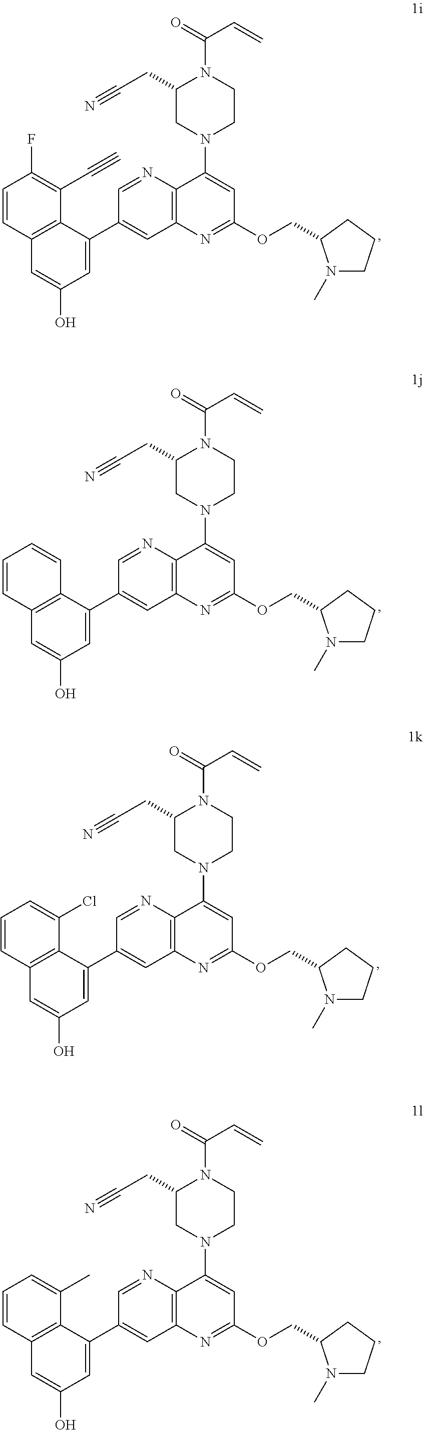

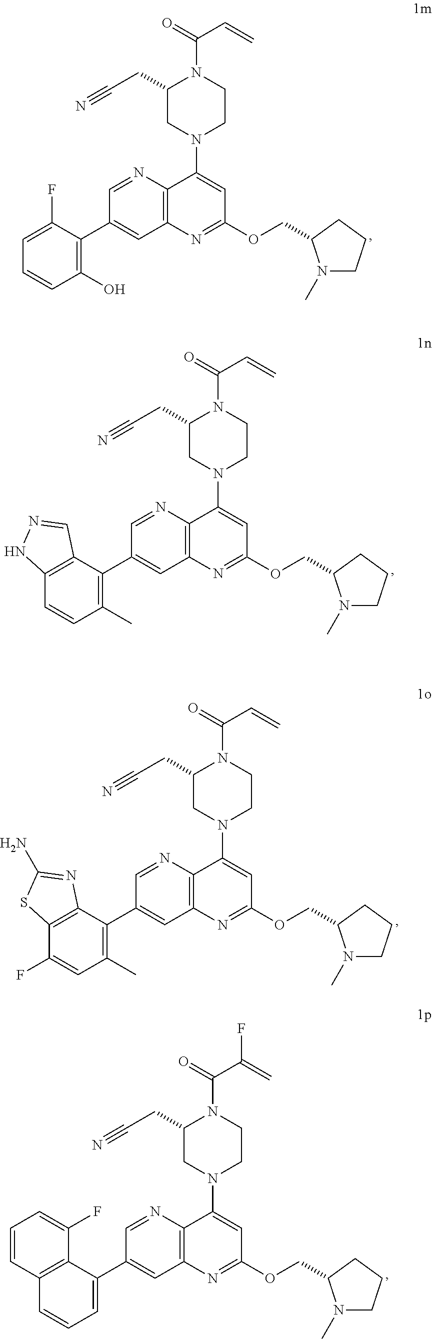

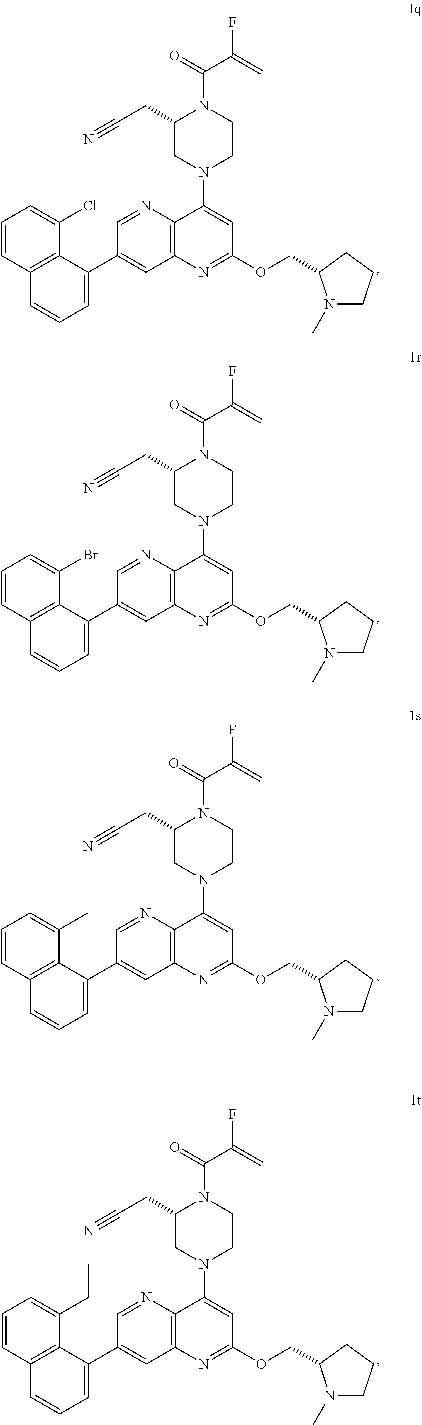

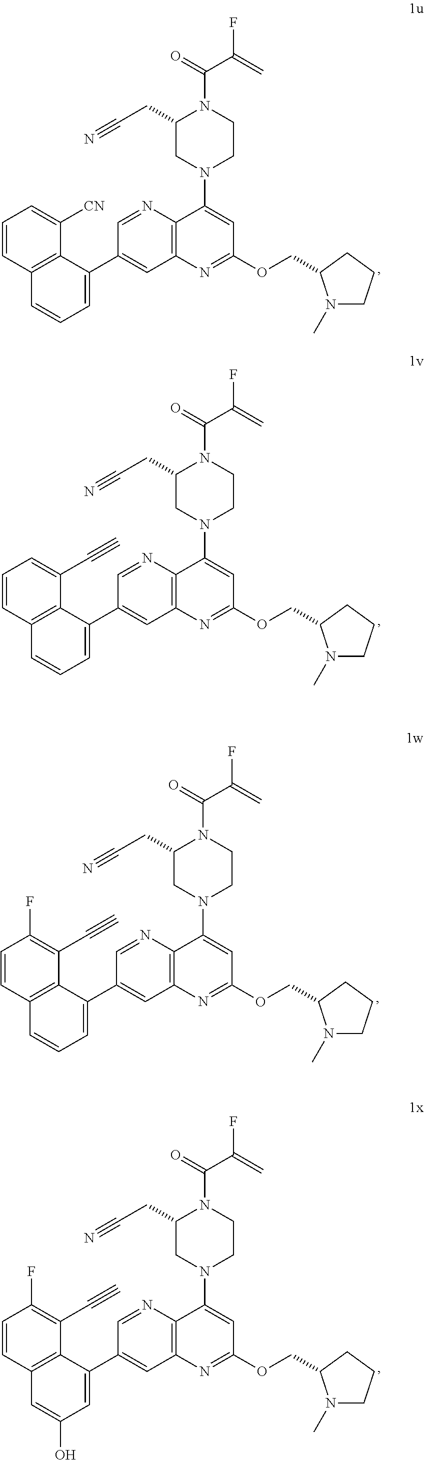

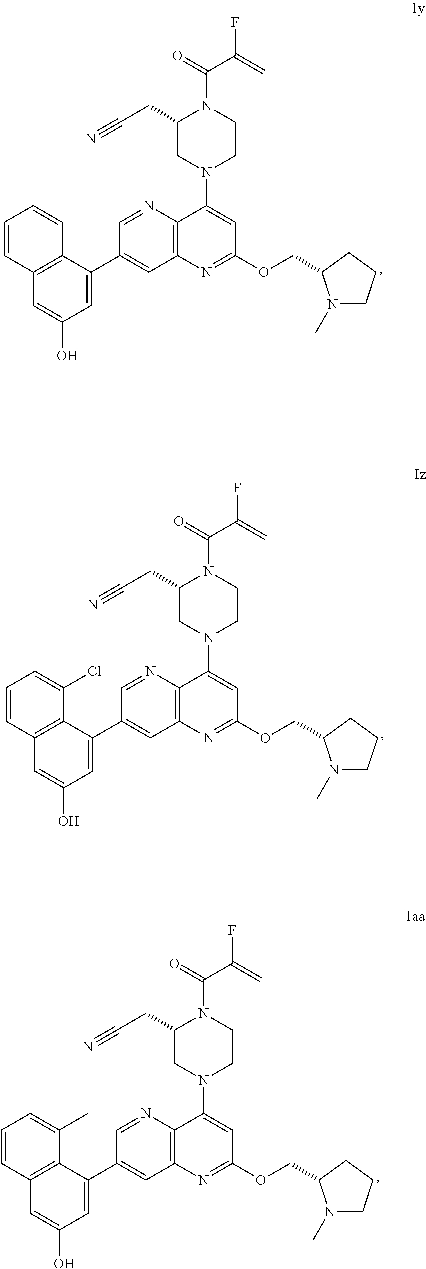

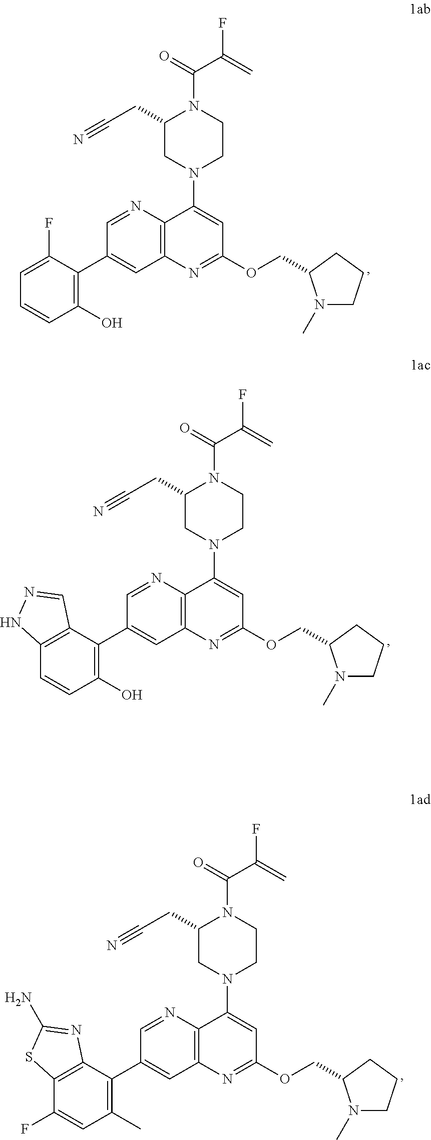

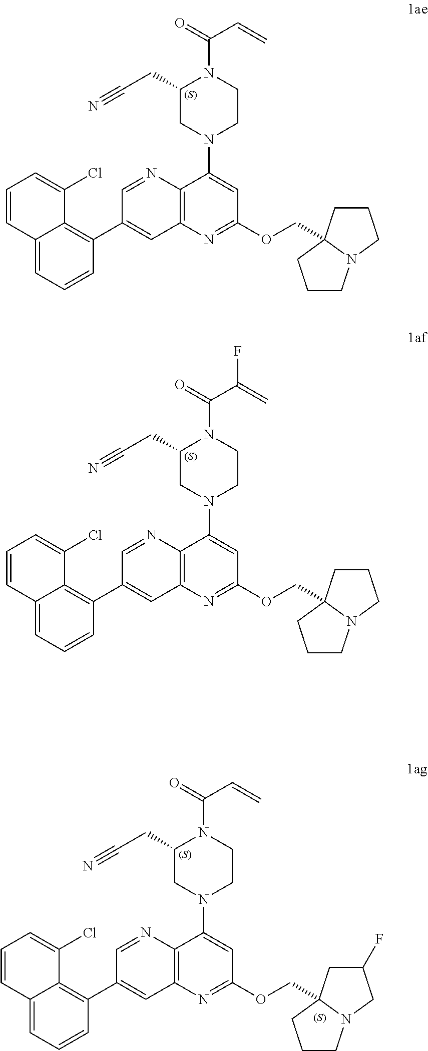

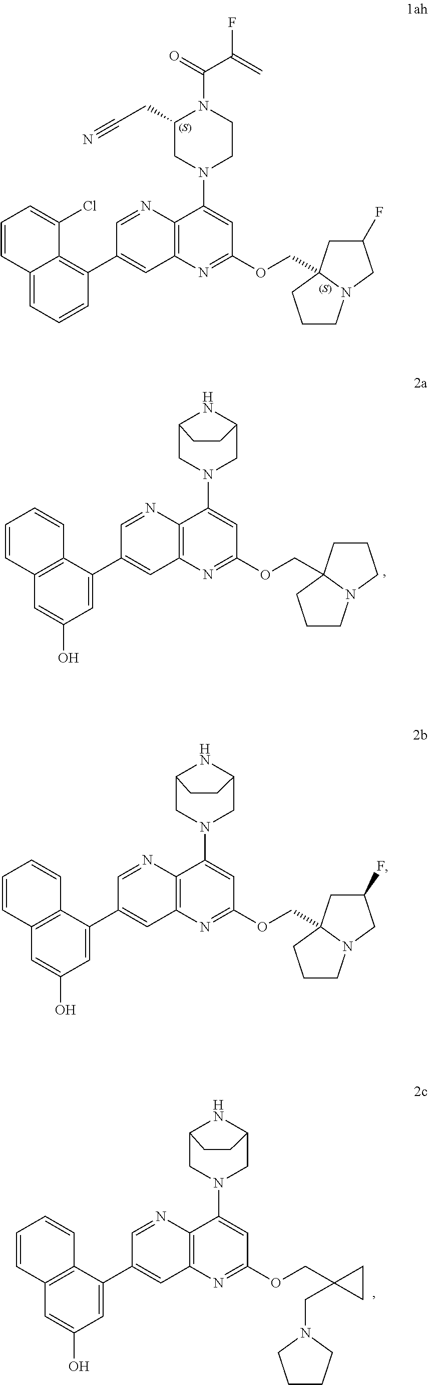

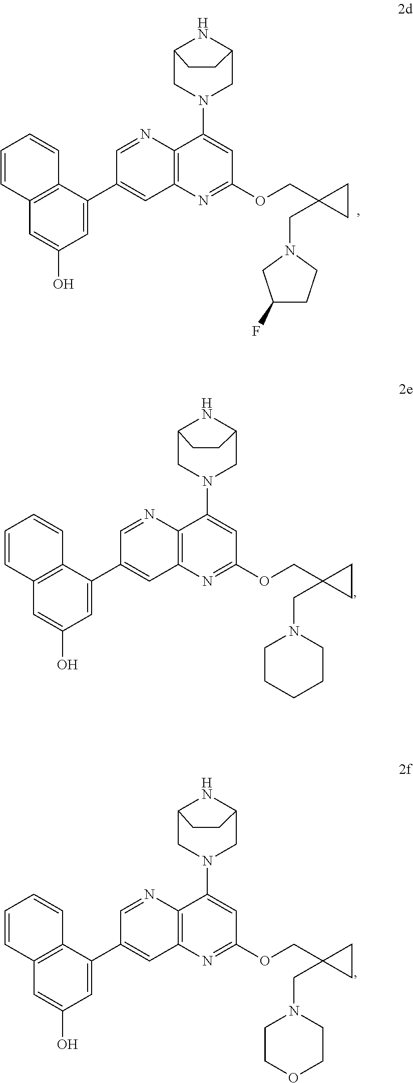 [SMILES: Oc1cc(-c2cnc3c(N4CC5CCC(C4)N5)cc(OCC4(CN5CC[C@@H](F)C5)CC4)nc3c2)c2ccccc2c1 ; Oc1cc(-c2cnc3c(N4CC5CCC(C4)N5)cc(OCC4(CN5CCCCC5)CC4)nc3c2)c2ccccc2c1 ; Oc1cc(-c2cnc3c(N4CC5CCC(C4)N5)cc(OCC4(CN5CCOCC5)CC4)nc3c2)c2ccccc2c1]

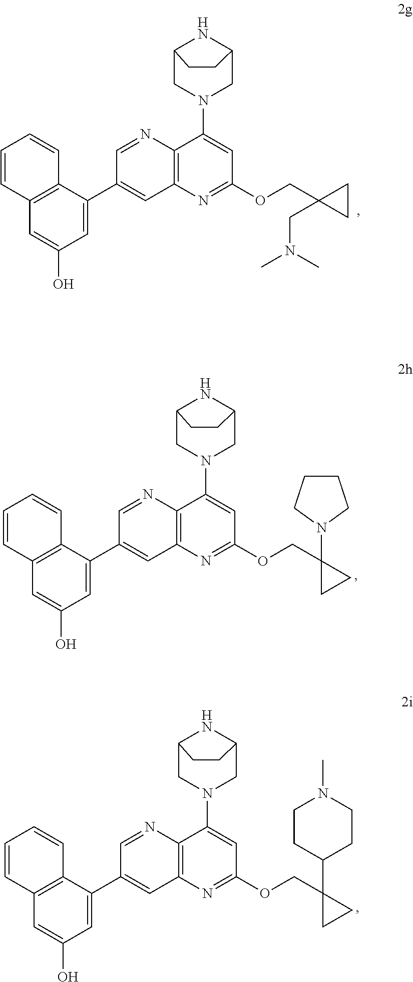 [SMILES: CN(C)CC1(COc2cc(N3CC4CCC(C3)N4)c3ncc(-c4cc(O)cc5ccccc45)cc3n2)CC1 ; Oc1cc(-c2cnc3c(N4CC5CCC(C4)N5)cc(OCC4(N5CCCC5)CC4)nc3c2)c2ccccc2c1 ; CN1CCC(C2(COc3cc(N4CC5CCC(C4)N5)c4ncc(-c5cc(O)cc6ccccc56)cc4n3)CC2)CC1]

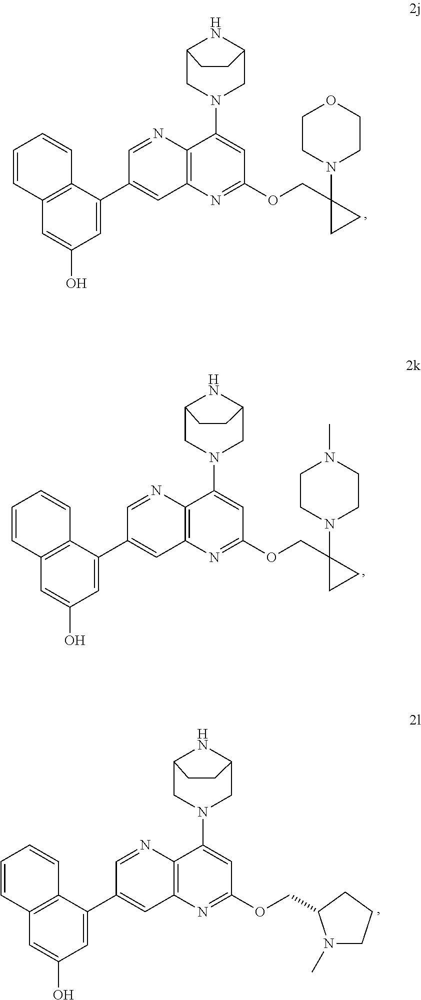

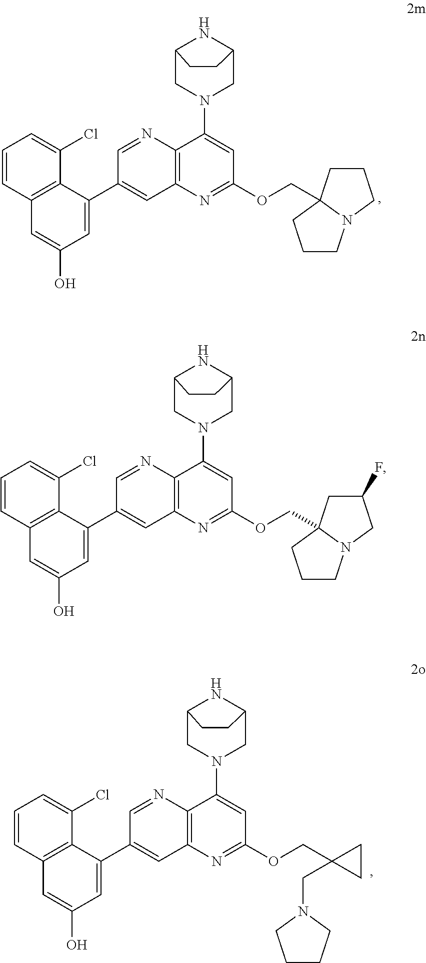

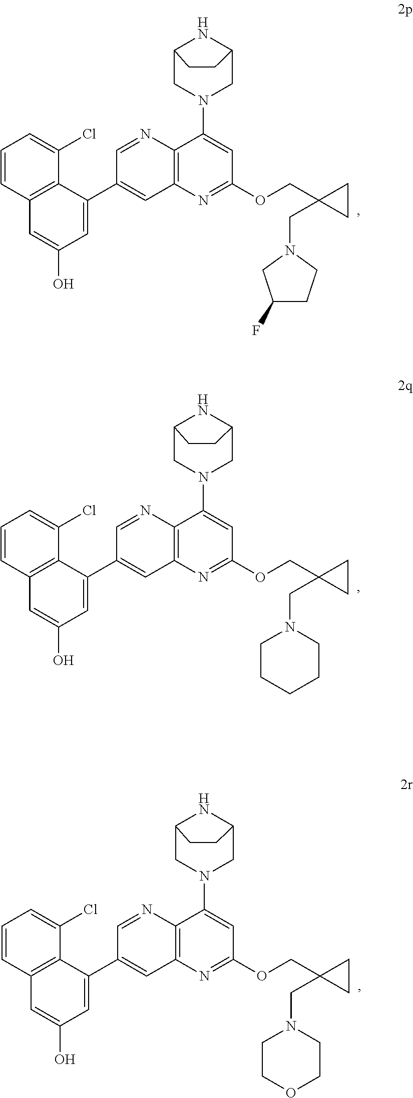 [SMILES: Oc1cc(-c2cnc3c(N4CC5CCC(C4)N5)cc(OCC4(CN5CC[C@@H](F)C5)CC4)nc3c2)c2c(Cl)cccc2c1 ; Oc1cc(-c2cnc3c(N4CC5CCC(C4)N5)cc(OCC4(CN5CCCCC5)CC4)nc3c2)c2c(Cl)cccc2c1 ; Oc1cc(-c2cnc3c(N4CC5CCC(C4)N5)cc(OCC4(CN5CCOCC5)CC4)nc3c2)c2c(Cl)cccc2c1]

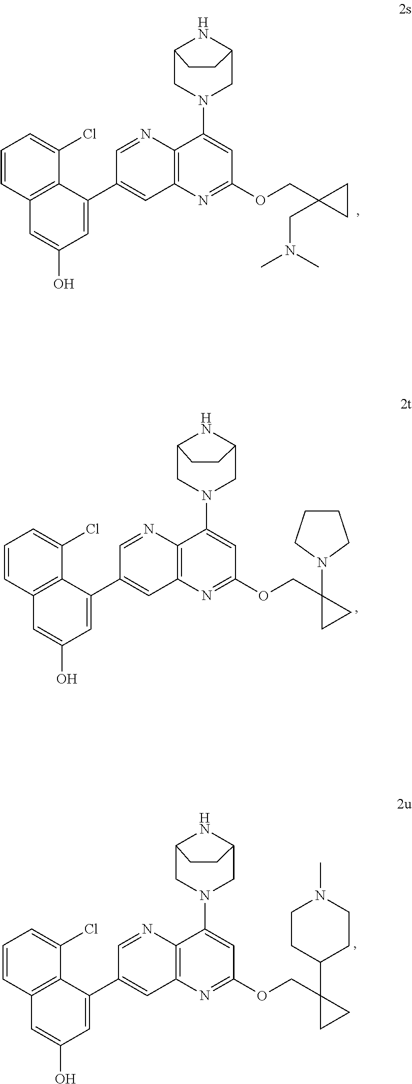 [SMILES: CN(C)CC1(COc2cc(N3CC4CCC(C3)N4)c3ncc(-c4cc(O)cc5cccc(Cl)c45)cc3n2)CC1 ; Oc1cc(-c2cnc3c(N4CC5CCC(C4)N5)cc(OCC4(N5CCCC5)CC4)nc3c2)c2c(Cl)cccc2c1 ; CN1CCC(C2(COc3cc(N4CC5CCC(C4)N5)c4ncc(-c5cc(O)cc6cccc(Cl)c56)cc4n3)CC2)CC1]

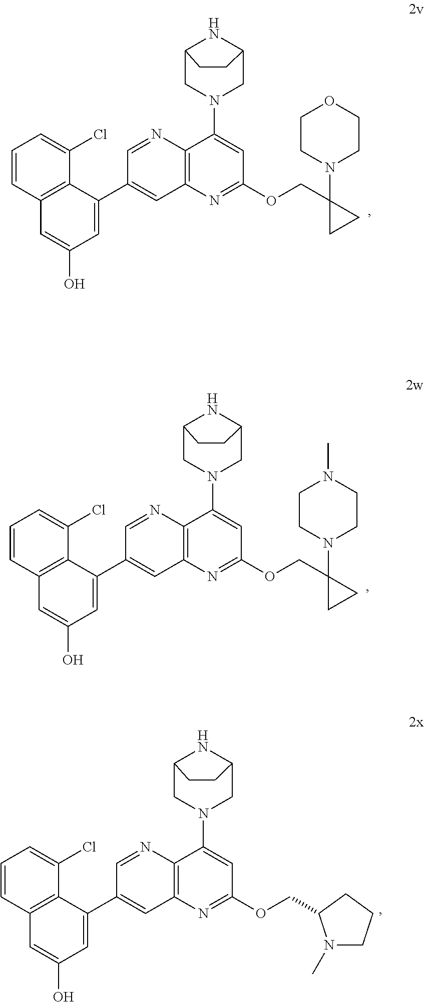

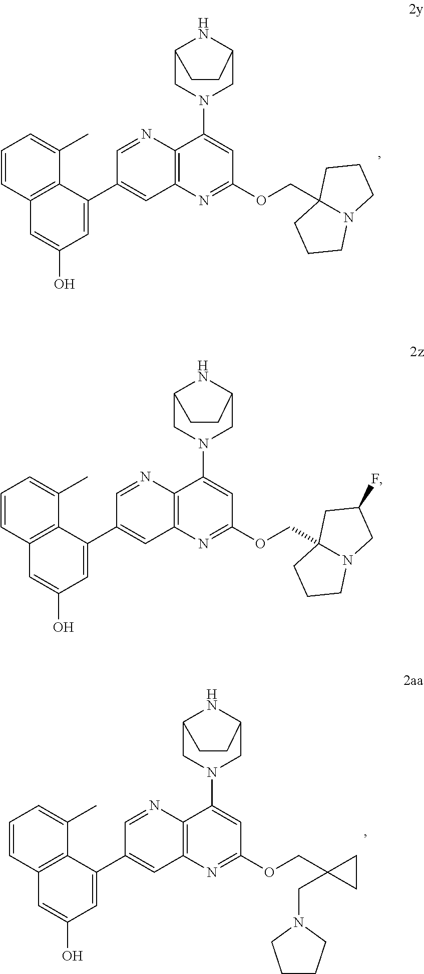 [SMILES: Cc1cccc2cc(O)cc(-c3cnc4c(N5CC6CCC(C5)N6)cc(OCC56CCCN5CCC6)nc4c3)c12 ; Cc1cccc2cc(O)cc(-c3cnc4c(N5CC6CCC(C5)N6)cc(OC[C@@]56CCCN5C[C@H](F)C6)nc4c3)c12 ; Cc1cccc2cc(O)cc(-c3cnc4c(N5CC6CCC(C5)N6)cc(OCC5(CN6CCCC6)CC5)nc4c3)c12]

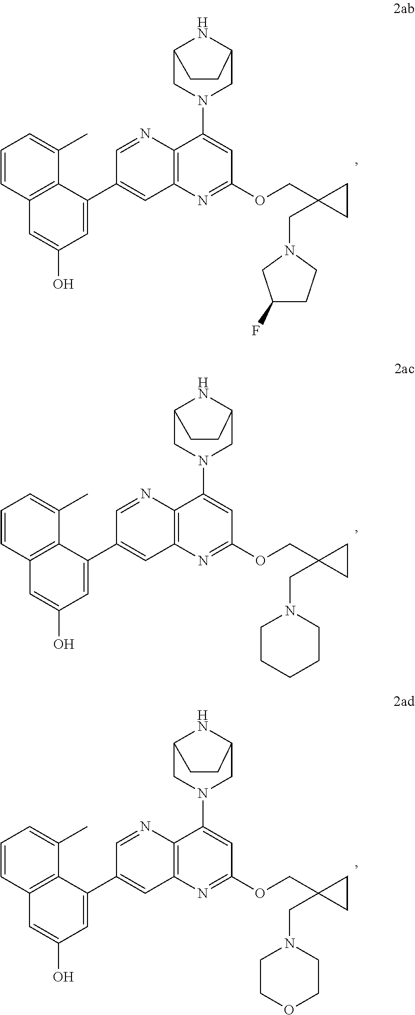 [SMILES: Cc1cccc2cc(O)cc(-c3cnc4c(N5CC6CCC(C5)N6)cc(OCC5(CN6CC[C@@H](F)C6)CC5)nc4c3)c12 ; Cc1cccc2cc(O)cc(-c3cnc4c(N5CC6CCC(C5)N6)cc(OCC5(CN6CCCCC6)CC5)nc4c3)c12 ; Cc1cccc2cc(O)cc(-c3cnc4c(N5CC6CCC(C5)N6)cc(OCC5(CN6CCOCC6)CC5)nc4c3)c12]

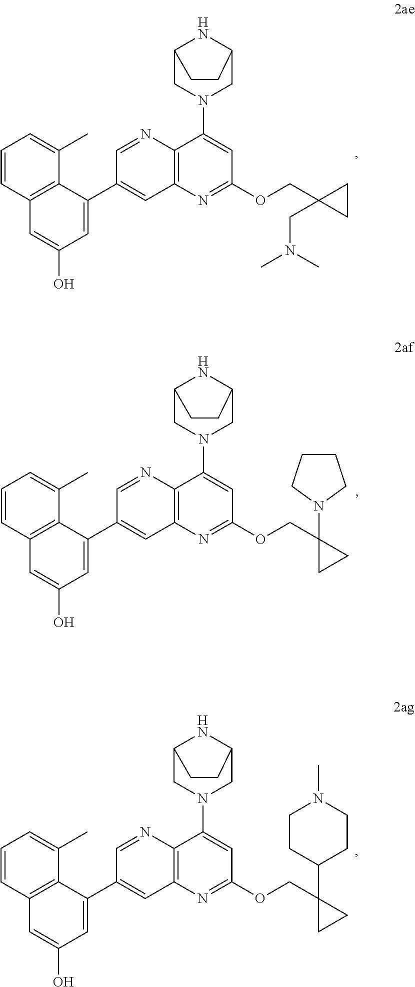 [SMILES: Cc1cccc2cc(O)cc(-c3cnc4c(N5CC6CCC(C5)N6)cc(OCC5(CN(C)C)CC5)nc4c3)c12 ; Cc1cccc2cc(O)cc(-c3cnc4c(N5CC6CCC(C5)N6)cc(OCC5(N6CCCC6)CC5)nc4c3)c12 ; Cc1cccc2cc(O)cc(-c3cnc4c(N5CC6CCC(C5)N6)cc(OCC5(C6CCN(C)CC6)CC5)nc4c3)c12]

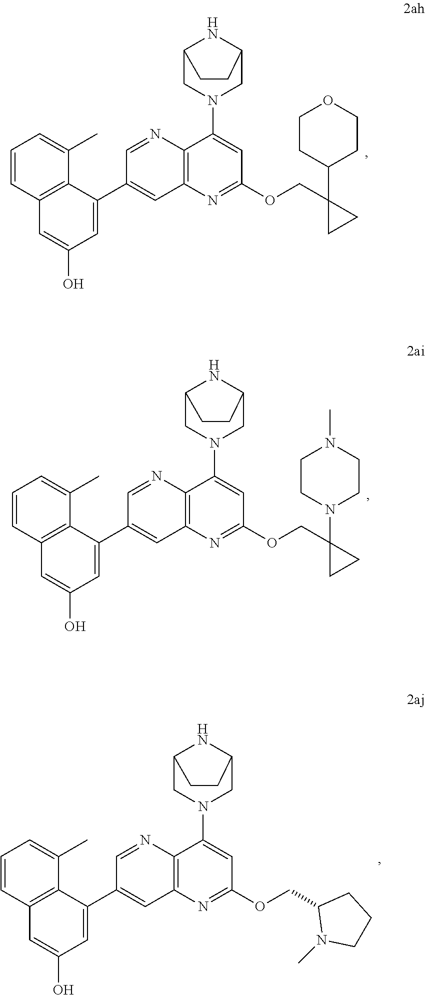

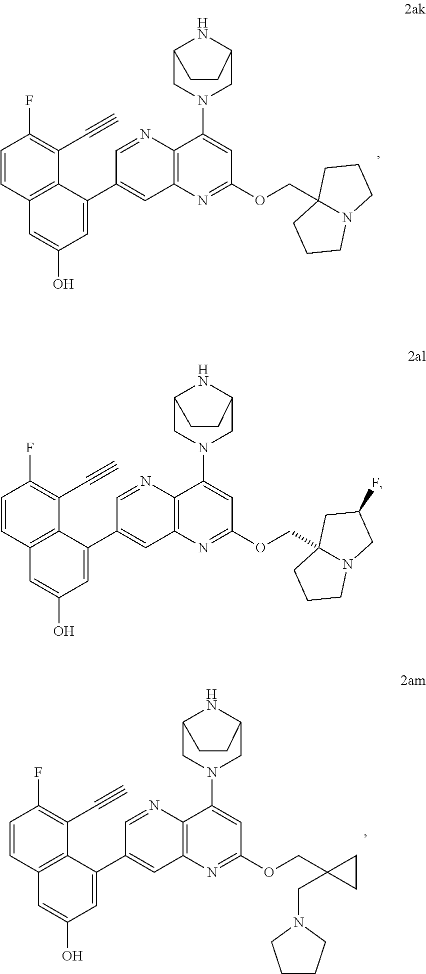 [SMILES: C#Cc1c(F)ccc2cc(O)cc(-c3cnc4c(N5CC6CCC(C5)N6)cc(OCC56CCCN5CCC6)nc4c3)c12 ; C#Cc1c(F)ccc2cc(O)cc(-c3cnc4c(N5CC6CCC(C5)N6)cc(OC[C@@]56CCCN5C[C@H](F)C6)nc4c3)c12 ; C#Cc1c(F)ccc2cc(O)cc(-c3cnc4c(N5CC6CCC(C5)N6)cc(OCC5(CN6CCCC6)CC5)nc4c3)c12]

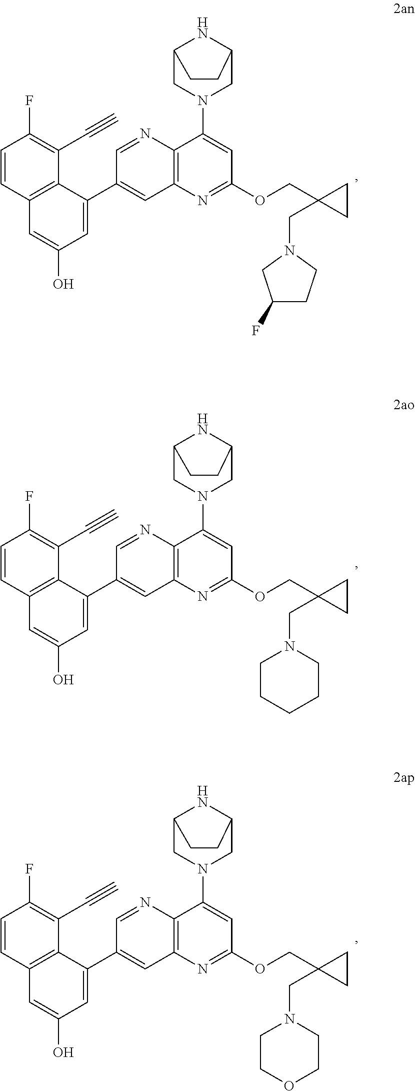 [SMILES: C#Cc1c(F)ccc2cc(O)cc(-c3cnc4c(N5CC6CCC(C5)N6)cc(OCC5(CN6CC[C@@H](F)C6)CC5)nc4c3)c12 ; C#Cc1c(F)ccc2cc(O)cc(-c3cnc4c(N5CC6CCC(C5)N6)cc(OCC5(CN6CCCCC6)CC5)nc4c3)c12 ; C#Cc1c(F)ccc2cc(O)cc(-c3cnc4c(N5CC6CCC(C5)N6)cc(OCC5(CN6CCOCC6)CC5)nc4c3)c12]

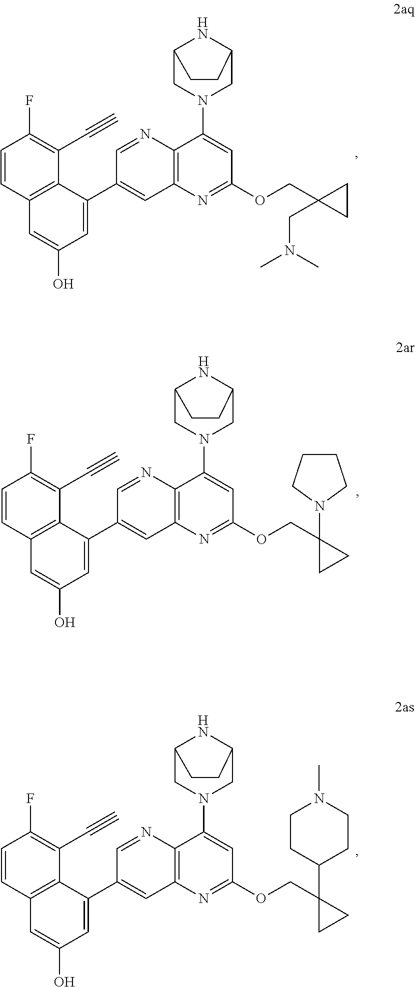 [SMILES: C#Cc1c(F)ccc2cc(O)cc(-c3cnc4c(N5CC6CCC(C5)N6)cc(OCC5(CN(C)C)CC5)nc4c3)c12 ; C#Cc1c(F)ccc2cc(O)cc(-c3cnc4c(N5CC6CCC(C5)N6)cc(OCC5(N6CCCC6)CC5)nc4c3)c12 ; C#Cc1c(F)ccc2cc(O)cc(-c3cnc4c(N5CC6CCC(C5)N6)cc(OCC5(C6CCN(C)CC6)CC5)nc4c3)c12]

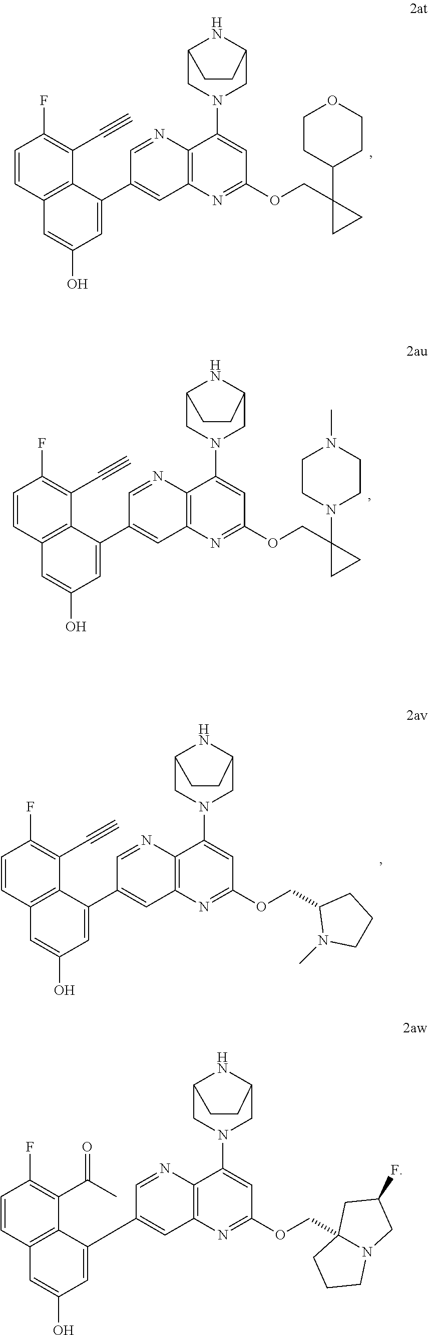

Particularly preferred compounds shown above are 1ae, 1af, 1ag, 1ah, 1b, 1d, 1i, 1q, 1s, 1x, 2z, 2al, 2an, 2ap and 2aq.

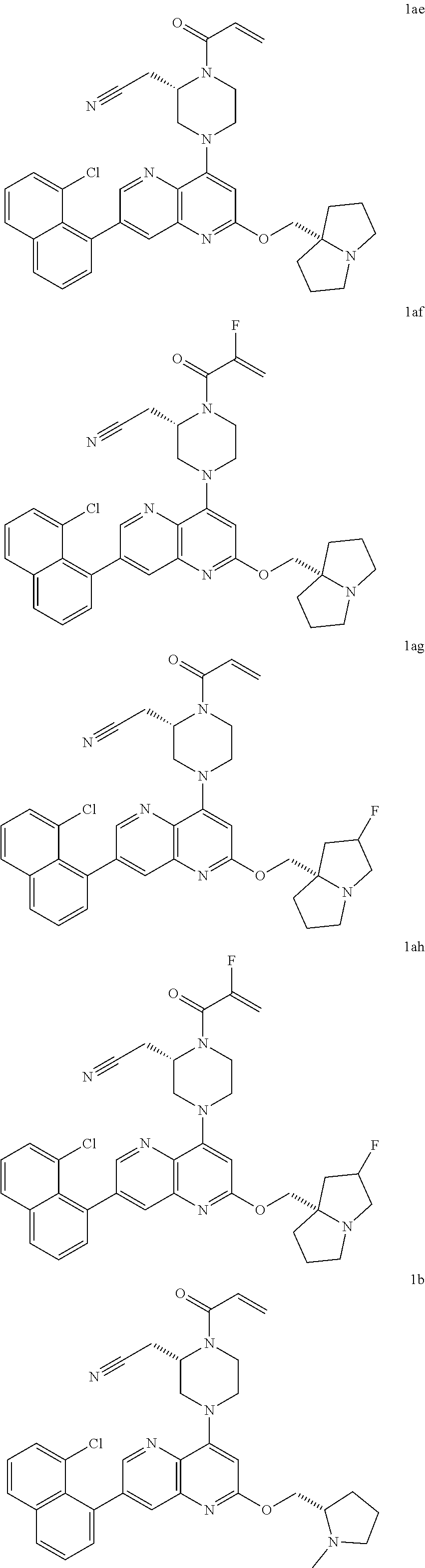

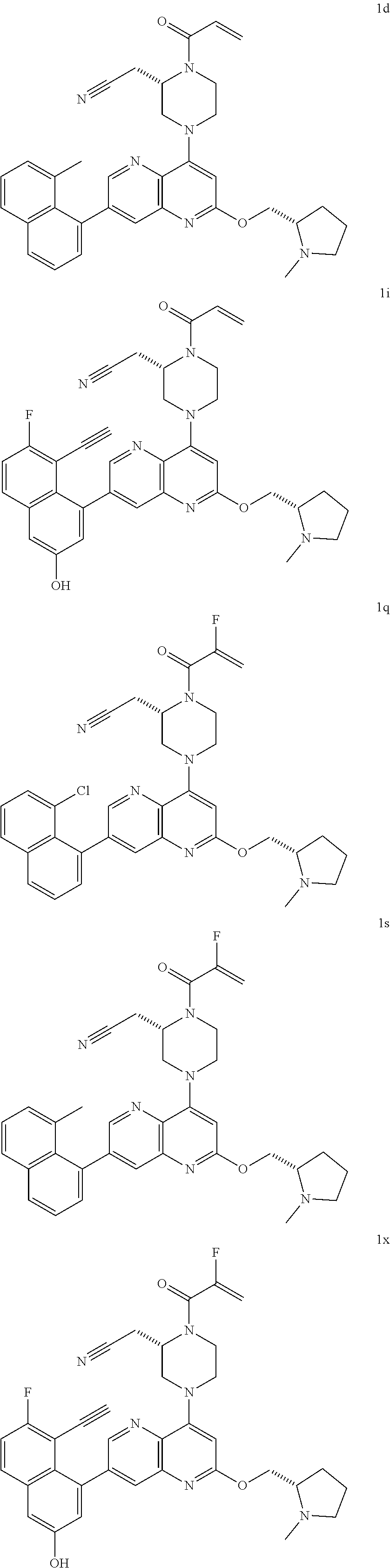

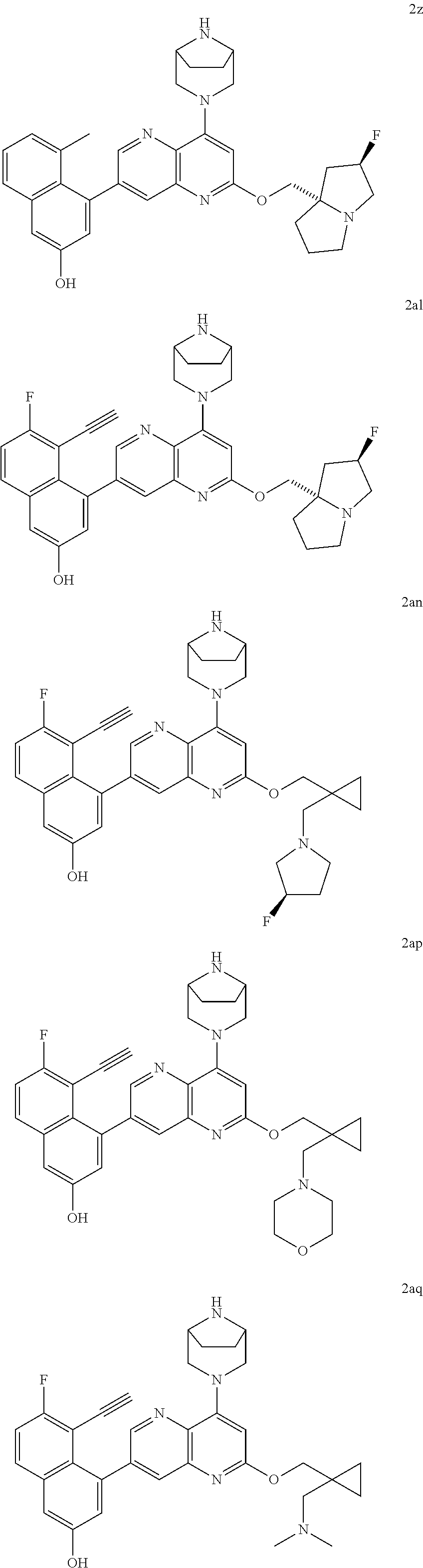

Compounds disclosed herein can be administered to a patient as the neat or freebase chemical, but are preferably administered as a pharmaceutical composition. Accordingly, the invention encompasses pharmaceutical compositions comprising a compound or a salt (including a pharmaceutically acceptable salt) of a compound, such as a compound of Formula I, together with at least one pharmaceutically acceptable carrier. The pharmaceutical composition may contain a compound or salt of Formula I as the only active agent, but is preferably contains at least one additional active agent. In certain embodiments the pharmaceutical composition is in a dosage form that contains from about 0.1 mg to about 2000 mg, from about 10 mg to about 1000 mg, from about 100 mg to about 800 mg, or from about 200 mg to about 600 mg of a compound of Formula I and optionally from about 0.1 mg to about 2000 mg, from about 10 mg to about 1000 mg, from about 100 mg to about 800 mg, or from about 200 mg to about 600 mg of an additional active agent in a unit dosage form. The pharmaceutical composition may also include a molar ratio of a compound, such as a compound of Formula I, and an additional active agent. For example, the pharmaceutical composition may contain a molar ratio of about 0.5:1, about 1:1, about 2:1, about 3:1 or from about 1.5:1 to about 4:1 of an additional active agent to a compound of Formula I. Particularly preferred forms of Formula I for use in a pharmaceutical composition include compounds 1ae-1ah, 1b, 1d, 1q, 2z, or 2al or a salt, solvate or prodrug thereof, together with a pharmaceutically acceptable carrier.

Compounds disclosed herein may be administered orally, topically, parenterally, by inhalation or spray, sublingually, transdermally, via buccal administration, rectally, as an ophthalmic solution, or by other means, in dosage unit formulations containing conventional pharmaceutically acceptable carriers. The pharmaceutical composition may be formulated as any pharmaceutically useful form, e.g., as an aerosol, a cream, a gel, a pill, a capsule, a tablet, a syrup, a transdermal patch, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin, talc, and vegetable oils. Optional active agents may be included in a pharmaceutical composition, which do not substantially interfere with the activity of the compound of the present invention.

The pharmaceutical compositions/combinations can be formulated for oral administration. These compositions contain between 0.1 and 99 weight % (wt %) of a compound of Formula I and usually at least about 5 wt % of a compound of Formula I. Some embodiments contain from about 25 wt % to about 50 wt % or from about 5 wt % to about 75 wt % of the compound of Formula I.

### Treatment Methods

The compounds of Formula I, as well as pharmaceutical compositions comprising the compounds, are useful for diagnosis or treatment of a disease, disorder, or medical condition mediated through KRAS, especially the KRAS mutants G12C and G12D, and including various cancers, such as glioma (glioblastoma), acute myelogenous leukemia, acute myeloid leukemia, myelodysplastic/myeloproliferative neoplasms, sarcoma, chronic myelomonocytic leukemia, non-Hodgkin lymphoma, astrocytoma, melanoma, non-small cell lung cancer, cholangiocarcinomas, chondrosarcoma, colon cancer or pancreatic cancer.

According to the present invention, a method of KRAS-mediated diseases or conditions comprises providing to a patient in need of such treatment a therapeutically effective amount of a compound of Formula I. In one embodiment, the patient is a mammal, and more specifically a human. As will be understood by one skilled in the art, the invention also encompasses methods of treating non-human patients such as companion animals, e.g. cats, dogs, and livestock animals.

A therapeutically effective amount of a pharmaceutical composition is preferably an amount sufficient to reduce or ameliorate the symptoms of a disease or condition. In the case of KRAS-mediated diseases for example, a therapeutically effective amount may be an amount sufficient to reduce or ameliorate cancer. A therapeutically effective amount of a compound or pharmaceutical composition described herein will also provide a sufficient concentration of a compound of Formula I when administered to a patient. A sufficient concentration is preferably a concentration of the compound in the patient's body necessary to prevent or combat the disorder. Such an amount may be ascertained experimentally, for example by assaying blood concentration of the compound, or theoretically, by calculating bioavailability.

According to the invention, the methods of treatment disclosed herein include providing certain dosage amounts of a compound of Formula I to a patient. Dosage levels of each compound of from about 0.1 mg to about 140 mg per kilogram of body weight per day are useful in the treatment of the above-indicated conditions (about 0.5 mg to about 7 g per patient per day). The amount of compound that may be combined with the carrier materials to produce a single dosage form will vary depending upon the patient treated and the particular mode of administration. Dosage unit forms will generally contain between from about 1 mg to about 500 mg of each active compound. In certain embodiments 25 mg to 500 mg, or 25 mg to 200 mg of a compound of Formula I are provided daily to a patient. Frequency of dosage may also vary depending on the compound used and the particular disease treated. However, for treatment of most KRAS-mediated diseases and disorders, a dosage regimen of 4 times daily or less can be used and in certain embodiments a dosage regimen of 1 or 2 times daily is used.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

A compound of Formula I may be administered singularly (i.e., sole therapeutic agent of a regime) to treat or prevent KRAS-mediated diseases and conditions such as various cancers, or may be administered in combination with another active agent. One or more compounds of Formula I may be administered in coordination with a regime of one or more other active agents such as anticancer cytotoxic agents. In an embodiment, a method of treating or diagnosing KRAS-mediated cancer in a mammal includes administering to said mammal a therapeutically effective amount of a compound of Formula I, optionally in combination with one or more additional active ingredients.

As will be appreciated by one skilled in the art, the methods of treatment provided herein are also useful for treatment of mammals other than humans, including for veterinary applications such as to treat horses and livestock, e.g., cattle, sheep, cows, goats, swine and the like, and pets (companion animals) such as dogs and cats.

For diagnostic or research applications, a wide variety of mammals will be suitable subjects including rodents (e.g., mice, rats, hamsters), rabbits, primates, and swine such as inbred pigs and the like. Additionally, for in vitro applications, such as in vitro diagnostic and research applications, body fluids (e.g., blood, plasma, serum, cellular interstitial fluid, saliva, feces, and urine) and cell and tissue samples of the above subjects will be suitable for use.

In one embodiment, the invention provides a method of treating a disease, disorder, or medical condition mediated through KRAS, especially the KRAS mutant G12C, including various cancers, in a patient identified as in need of such treatment, the method comprising providing to the patient an effective amount of a compound of Formula I. The compounds of Formula I provided herein may be administered alone, or in combination with one or more other active agents.

In another embodiment, the method of treating or diagnosing KRAS-mediated diseases or conditions may additionally comprise administering the compound of Formula I in combination with one or more additional compounds, wherein at least one of the additional compounds is an active agent, to a patient in need of such treatment. The one or more additional compounds may include additional therapeutic compounds, including anticancer therapeutic compounds such as doxorubicin, paclitaxel, docetaxel, cisplatin, camptothecin, temozolomide, avastin, Herceptin, Erbitux, EGFR inhibitors, osimertinib, rezivertinib, CDK 4/6 inhibitors, abemaciclib, palbociclib, ribociclib, c-MET inhibitors, capmatinib, volitinib, ALK inhibitors, crizotinib, alectinib, ceritinib, brigatinib, entrectinib, lorlatinib, PD-1 antagonists, PD-L1 antagonists, ipilimumab, embrolizumab, nivolumab, and the like.

### EXAMPLES

### Chemical Synthesis

The compounds of the Formula 1 described herein, and/or the pharmaceutically acceptable salts thereof, can be synthesized from commercially available starting materials by methods well known to those skilled in the art of synthetic organic chemistry. The following general synthetic Schemes 1 and 2 illustrate representative methods to prepare most of the example compounds. In the specific examples where the Suzuki cross-coupling reaction of arylboronic acids/esters with organohalides/pseudohalides (Beketskaya, I. P. et al., Coordin. Chem. Rev. 2019, 385, 137-173) is either impractical or unsuccessful, then the corresponding Stille cross-coupling reaction of organostannanes with organohalides/pseudohalides may be used as an alternative (Espinet, P. et al., ACS Catal. 2015, 5, 3040-3053). Many of the requisite intermediates can be prepared as described in WO2021041671. The listed starting materials, reactions, reagents, solvents, temperatures, catalysts and ligands are not limited to what is depicted for purely illustrative purposes. Certain abbreviations and acronyms well known to those trained in the art that may be used in Schemes 1 and 2 as well as in the Examples are listed below for clarity.

The synthesis of compounds of this invention is exemplified by the sequence of steps shown in Scheme 1. In Scheme 1, oxidation of 1,5-naphthyridine derivative 3 with mCPBA in a solvent such as CH₂Cl₂ generates N-oxide compound 4. Reaction of 4 with POCl₃ at an elevated temperature furnishes the corresponding chloro derivative 5. Reaction of 5 with 6 yields compound 7. Treatment of 8 with an appropriate base such as sodium hydride, Hünig's base, K₂CO₃ or a Cs₂CO₃/DABCO mixture followed by reaction with 7 in a polar aprotic solvent such as N-methyl-2-pyrrolidone at RT or elevated temperature generates compound 9. Suzuki-Miyaura coupling of 9 with a boronic ester such as 10 (or the corresponding boronic acid) under standard conditions in solvent mixture such as 1,4-dioxane and water can be employed to prepare 11. Removal of the Boc protecting group of 11 under acidic conditions such as anhydrous 4M HCl in 1,4-dioxane or TFA in CH₂Cl₂ generates compounds of the Formula I where R² is hydrogen (12). Acylation of 12 with acryloyl chloride 13 in a solvent such as methylene chloride containing a base such as triethylamine, will produce the corresponding compounds 14 of the Formula I where R² is either —C(O)CH═CH, —C(O)CF═CH or —C(O)CCl═CH.

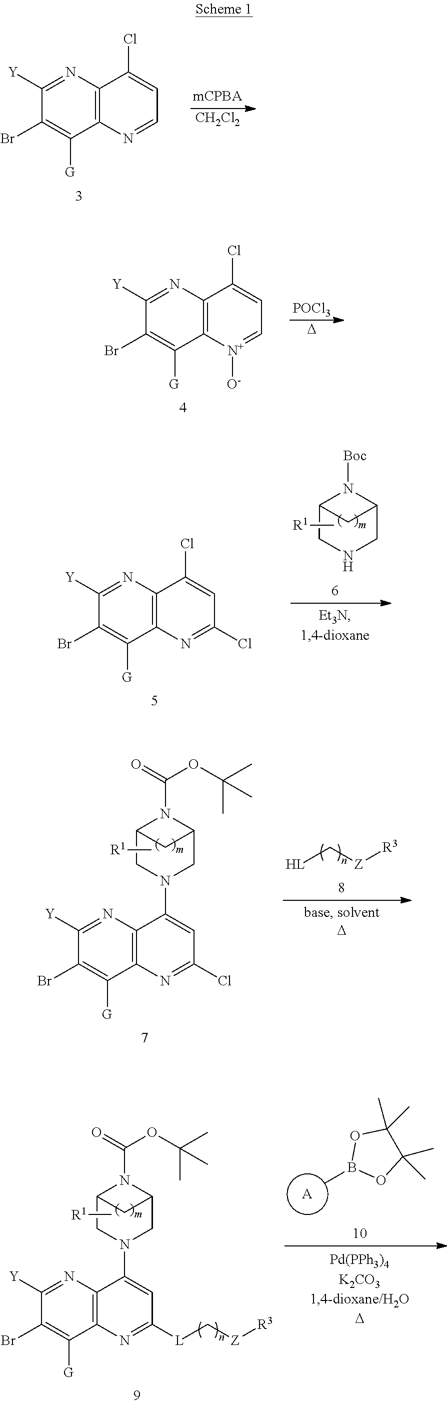

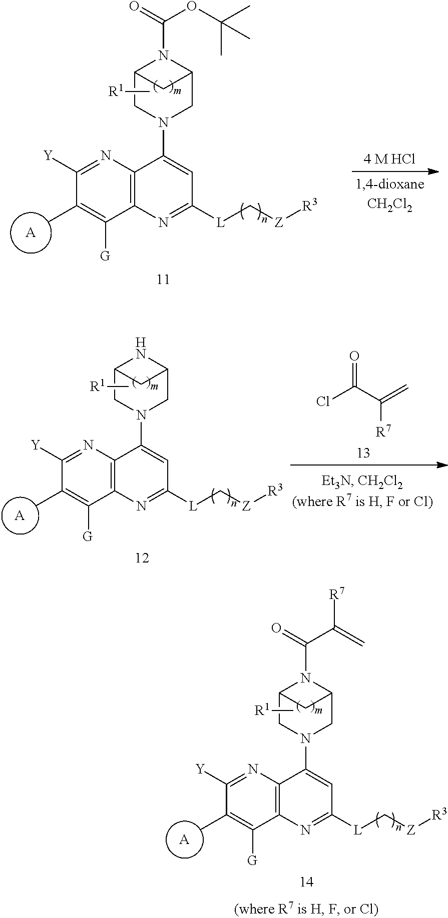

### Abbreviations and Acronyms

The following abbreviations and acronyms may be used in this application:
anhyd.=anhydrous;
aq.=aqueous;
B₂pin₂=bis(pinacolato)diboron;
Boc=tert-butoxycarbonyl;
n-Bu₃P=tri-n-butylphosphine;
Compd=compound;
d=day(s);
DCM=dichloromethane;
DIEA=DIPEA=N,N-diisopropylethylamine;
DMF=N,N-dimethylformamide;
DMSO=dimethylsulfoxide;
DMA=N,N-dimethylacetamide;
dppf=1,1′-bis(diphenylphosphino) ferrocene);
DTBPF=1,1′-bis(di-tert-butylphosphino) ferrocene;
EtOAc=ethyl acetate;
equiv=equivalents;
Ex=Example;
h=hour(s);
KOAc=potassium acetate;
LiHMDS=lithium bis(trimethylsilyl)amide [LiN(SiMe₃)_{2];}
mCPBA=meta-chloroperoxybenzoic acid:
MeOH=methanol;
NMP=N-methyl-2-pyrrolidone;
min=minutes;
Pd(dppf)Cl₂=[1,1′-bis(diphenylphosphino)ferrocene]dichloropalladium(II);
RT=room temperature;
satd.=saturated solution;
TEA=triethylamine;
TFA=trifluoroacetic acid;
THF=tetrahydrofuran;

The present inventive concept has been described in terms of exemplary principles and embodiments, but those skilled in the art will recognize that variations may be made and equivalents substituted for what is described without departing from the scope and spirit of the disclosure as defined by the following claims.

### Example 1

### 2-((S)-1-Acryloyl-4-(7-(8-chloronaphthalen-1-yl)-2-(((S)-1-methylpyrrolidin-2-yl)methoxy)-1,5-naphthyridin-4-yl)piperazin-2-yl)acetonitrile (1b)

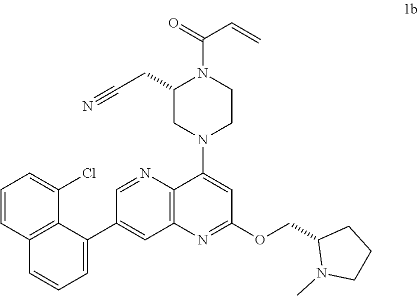

### Example 1 (1B) was Prepared as Shown Below in Scheme 2

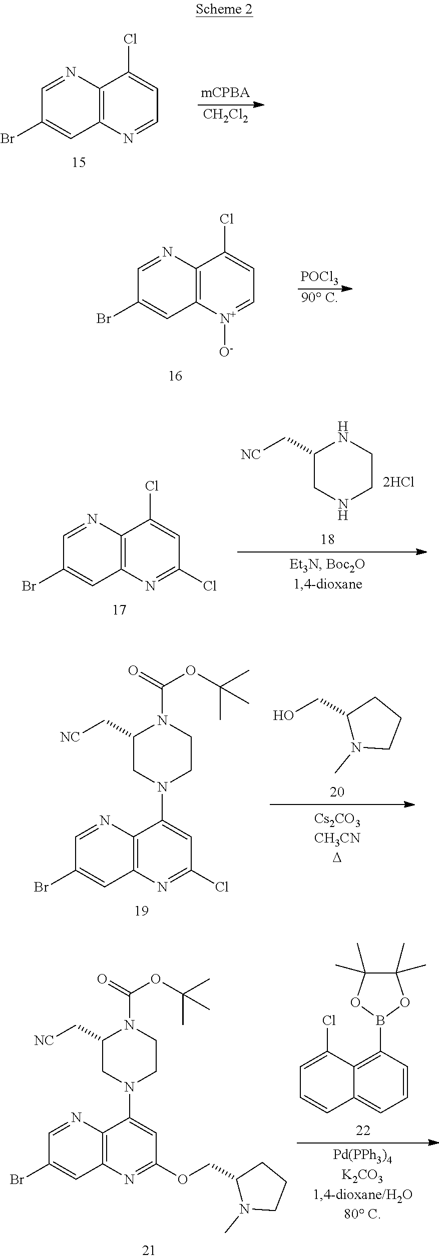

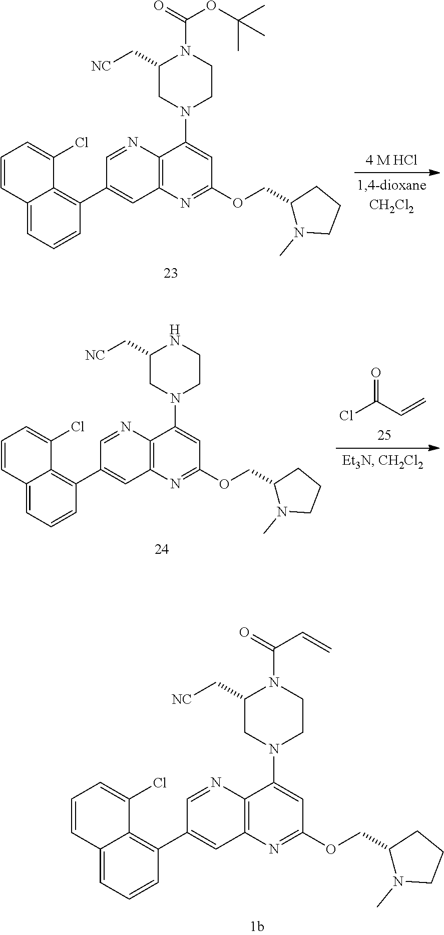

7-Bromo-4-chloro-1,5-naphthyridine-1-oxide (16). This compound was prepared as described on pages 64-65 in WO2020150114 from the reaction of mCPBA and 3-bromo-8-chloro-1,5-naphthyridine (15; CAS #97267-61-3; 1.70 g, 7.02 mmol) in CH₂Cl₂ to afford 1.50 g (83%) of 7-bromo-4-chloro-1,5-naphthyridine-1-oxide (16) as a pale-yellow solid: HPLC-MS (ES⁺) m/z [M+H⁺]=259, 261, 263; ¹H NMR (300 MHz, CDCl₃) δ 9.24 (d, J=2.2 Hz, 1H), 9.12 (d, J=2.2 Hz, 1H), 8.44 (d, J=6.7 Hz, 1H), 7.63 (d, J=6.7 Hz, 1H).

7-Bromo-2,4-dichloro-1,5-naphthyridine (17). This compound was prepared as described on page 65 in WO2020150114 from 7-bromo-4-chloro-1,5-naphthyridine-1-oxide (16; 775 mg, 3.00 mmol) to afford 750 mg (90%) of 7-bromo-2,4-dichloro-1,5-naphthyridine (17) as a pink solid: HPLC-MS (ES⁺) m/z [M+H⁺]=277, 279, 281, 283; ¹H NMR (300 MHz, CDCl₃) δ 9.05 (d, J=2.1 Hz, 1H), 8.51 (d, J=2.1 Hz, 1H), 7.78 (s, 1H).

tert-Butyl(S)-4-(7-bromo-2-chloro-1,5-naphthyridin-4-yl)-2-(cyanomethyl)piperazine-1-carboxylate (19). Triethylamine (1.90 mL, 13.6 mmol) was added to a suspension of(S)-2-(piperazin-2-yl)acetonitrile dihydrochloride (18; CAS #1589082-26-7; 538 mg, 2.72 mmol) in anhydrous 1,4-dioxane (10 mL) and stirred at RT. After 5 h, the mixture was cooled to 0° C. and anhydrous 1,4-dioxane (20 mL) was added followed by the portionwise addition of 7-bromo-2,4-dichloro-1,5-naphthyridine (17; 750 mg, 2.72 mmol). After complete addition, the ice bath was removed, warmed to RT and after 5 minutes the reaction mixture was heated at reflux for 23 h. The mixture was cooled to RT and di-tert-butyl dicarbonate (1.87 mL, 8.16 mmol) was added. After 16 h, the mixture was diluted with EtOAc, washed with satd. aq. NaCl (3×), dried (MgSO₄), filtered, and concentrated in vacuo. The crude product was purified by silica gel column chromatography eluting with a gradient of 10% to 40% EtOAc in hexane to afford 610 mg (48%) of tert-butyl(S)-4-(7-bromo-2-chloro-1,5-naphthyridin-4-yl)-2-(cyanomethyl)piperazine-1-carboxylate (19) as a white solid: HPLC-MS (ES⁺) m/z [M+H⁺]=466, 468, 470; ¹H NMR (300 MHz, CDCl₃) δ 8.89 (d, J=2.2 Hz, 1H), 8.37 (d, J=2.2 Hz, 1H), 6.83 (s, 1H), 4.94 (d, J=12.6 Hz, 1H), 4.63 (br s, 1H), 4.18 (br s, 1H), 3.76-3.68 (m, 1H), 3.33-3.06 (m, 4H), 2.79 (dd, J=5.3, 10.9 Hz, 1H), 1.53 (s, 9H).

tert-Butyl(S)-4-(7-bromo-2-(((S)-1-methylpyrrolidin-2-yl)methoxy)-1,5-naphthyridin-4-yl)-2-(cyanomethyl)piperazine-1-carboxylate (21). A mixture of tert-butyl(S)-4-(7-bromo-2-chloro-1,5-naphthyridin-4-yl)-2-(cyanomethyl)piperazine-1-carboxylate (19; 390 mg, 0.839 mmol), (2S)-1-methyl-2-pyrrolidinemethanol (20; CAS #34381-71-0; 1.0 mL, 8.0 mmol), and Cs₂CO₃ (545 mg, 1.68 mmol) in anhydrous CH₃CN (8 mL) was heated at reflux. After 72 h, the mixture was cooled to RT, diluted with EtOAc, washed with satd. aq. NaCl (3×), dried (MgSO₄), filtered, and concentrated in vacuo. The crude product was purified by silica gel column chromatography eluting with a gradient of 0% to 15% MeOH in DCM to afford 80 mg (17%) of tert-butyl(S)-4-(7-bromo-2-(((S)-1-methylpyrrolidin-2-yl)methoxy)-1,5-naphthyridin-4-yl)-2-(cyanomethyl)piperazine-1-carboxylate as a light brown solid: HPLC-MS (ES⁺) m/z [M+H⁺]=545, 547; ¹H NMR (300 MHz, CDCl₃) δ 8.72 (d, J=2.2 Hz, 1H), 8.22 (d, J=2.2 Hz, 1H), 6.41 (s, 1H), 4.74 (d, J=12.5 Hz, 1H), 4.62 (br s, 1H), 4.48 (dd, J=4.8, 6.4 Hz, 1H), 4.36 (dd, J=4.8, 6.4 Hz, 1H), 4.15 (br s, 1H), 3.60 (d, J=5.8 Hz, 1H), 3.22 (br s, 1H), 3.32 (dd, J=7.7, 8.8 Hz, 1H), 3.14 (t, J=7.4 Hz, 1H), 2.98 (td, J=3.3, 9.1 Hz, 2H), 2.83 (dd, J=5.6, 10.7 Hz, 1H), 2.67-2.57 (m, 1H), 2.48 (s, 3H), 2.34-2.22 (m, 1H), 2.06-1.94 (m, 1H), 1.93-1.71 (m, 3H), 1.52 (s, 9H).

tert-Butyl(S)-4-(7-(8-chloronaphthalen-1-yl)-2-(((S)-1-methylpyrrolidin-2-yl)methoxy)-1,5-naphthyridin-4-yl)-2-(cyanomethyl)piperazine-1-carboxylate (23). A mixture of tert-butyl(S)-4-(7-bromo-2-(((S)-1-methylpyrrolidin-2-yl)methoxy)-1,5-naphthyridin-4-yl)-2-(cyanomethyl)piperazine-1-carboxylate (21; 106 mg, 0.195 mmol), 2-(8-chloronaphthalen-1-yl)-4,4,5,5-tetramethyl-1,3,2-dioxaborolane (22; 225 mg, 0.780 mmol), and K₂CO₃ (487 mg, 3.53 mmol) in 1,4-dioxane (3 mL) and water (1.4 mL) was degassed by sparging with N₂ with stirring for 30 minutes. Tetrakis(triphenylphosphine) palladium (0) (33 mg, 0.029 mmol) was added and the reaction mixture degassed by sparging with N₂ with stirring for an additional 20 minutes. The reaction mixture was heated at 80° C. with stirring under a N₂ atmosphere for 16 h. The reaction mixture was cooled to RT, diluted with EtOAc, filtered through Celite and the filtrate was then washed with satd. aq. NaCl (3×), dried (MgSO₄), filtered and concentrated in vacuo. The crude product was purified by silica gel column chromatography eluting with a gradient of 0% to 10% MeOH in DCM to afford 48 mg (39%) of tert-butyl(S)-4-(7-(8-chloronaphthalen-1-yl)-2-(((S)-1-methylpyrrolidin-2-yl)methoxy)-1,5-naphthyridin-4-yl)-2-(cyanomethyl)piperazine-1-carboxylate (23) as an off-white solid: HPLC-MS (ES⁺) m/z [M+H⁺]=627, 629; ¹H NMR (300 MHz, CDCl₃) δ 8.67 (d, J=2.1 Hz, 1H), 8.02 (t, J=2.0 Hz, 1H), 7.94 (d, J=7.8 Hz, 1H), 7.87 (d, J=7.5 Hz, 1H), 7.55 (t, J=7.8 Hz, 2H), 7.42 (t, J=7.4 Hz, 2H), 6.45 (d, J=2.5 Hz, 1H), 4.92 (d, J=11.6 Hz, 1H), 4.79 (d, J=13.8 Hz, 1H), 4.67 (br s, 1H), 4.58-4.48 (m, 1H), 4.44-4.35 (m, 1H), 4.20 (br s, 1H), 3.70 (br t, J=10.3 Hz, 1H), 3.46-3.21 (m, 2H), 3.19-2.88 (m, 4H), 2.63 (br s, 1H), 2.49 (s, 3H), 2.34-2.22 (m, 1H), 2.08-1.69 (m, 3H), 1.53 (s, 9H).

2-((S)-4-(7-(8-Chloronaphthalen-1-yl)-2-(((S)-1-methylpyrrolidin-2-yl)methoxy)-1,5-naphthyridin-4-yl)piperazin-2-yl)acetonitrile (24). A solution of tert-butyl(S)-4-(7-(8-chloronaphthalen-1-yl)-2-(((S)-1-methylpyrrolidin-2-yl)methoxy)-1,5-naphthyridin-4-yl)-2-(cyanomethyl)piperazine-1-carboxylate (23; 44 mg, 0.070 mmol) in CH₂Cl₂ (2 mL) was treated with a solution of 4 M HCl/1,4-dioxane (0.2 mL) and stirred at RT. After 16 h, the mixture was treated with 0.1 M NaOH solution until the pH was basic. The layers were separated and the aqueous layer was extracted with CH₂Cl₂ (3×), dried (MgSO₄), filtered, and concentrated in vacuo to afford 31 mg (84%) of 2-((S)-4-(7-(8-chloronaphthalen-1-yl)-2-(((S)-1-methylpyrrolidin-2-yl)methoxy)-1,5-naphthyridin-4-yl)piperazin-2-yl)acetonitrile (24) as an off-white solid: HPLC-MS (ES⁺) m/z [M+H⁺]=527, 529; ¹H NMR (300 MHz, CDCl₃) δ 8.64 (ddd, J=1.4, 2.2, 2.8 Hz, 1H), 8.04-7.99 (m, 1H), 7.95 (dd, J=1.2, 7.0 Hz, 1H), 7.88 (dd, J=1.2, 7.0 Hz, 1H), 7.62-7.49 (m, 2H), 7.47-7.37 (m, 2H), 6.46 (s, 1H), 4.59-4.47 (m, 1H), 4.44-4.32 (m, 1H), 4.29-3.84 (m, 2H), 3.80-3.73 (m, 1H), 3.68-3.60 (m, 1H), 3.57-3.44 (m, 1H), 3.32-2.84 (m, 4H), 2.74-2.56 (m, 2H), 2.49 (s, 3H), 2.43-2.20 (m, 2H), 2.10-1.47 (m, 4H).

2-((S)-1-Acryloyl-4-(7-(8-chloronaphthalen-1-yl)-2-(((S)-1-methylpyrrolidin-2-yl)methoxy)-1,5-naphthyridin-4-yl)piperazin-2-yl)acetonitrile (1b). A solution of 2-((S)-4-(7-(8-chloronaphthalen-1-yl)-2-(((S)-1-methylpyrrolidin-2-yl)methoxy)-1,5-naphthyridin-4-yl)piperazin-2-yl)acetonitrile (24; 50 mg, 0.095 mmol) in CH₂Cl₂ (8 mL) was treated with Et₃N (16 μL, 0.114 mmol) and stirred at RT. The mixture was cooled to 0° C. and treated with acryloyl chloride (25; CAS #814-68-6; 10 μL, 0.114 mmol). After 1 h at 0° C., the mixture was diluted with CH₂Cl₂ and washed with H₂O (2×). The layers were separated and the CH₂Cl₂ layer was dried (MgSO₄), filtered and concentrated in vacuo. The crude product was purified by silica gel column chromatography eluting with a gradient of 0% to 10% MeOH in DCM containing 5% NH₄OH (v/v) to afford 20 mg (36%) of 2-((S)-1-acryloyl-4-(7-(8-chloronaphthalen-1-yl)-2-(((S)-1-methylpyrrolidin-2-yl)methoxy)-1,5-naphthyridin-4-yl)piperazin-2-yl)acetonitrile (1b) as an off-white solid: HPLC-MS (ES⁺) m/z [M+H⁺]=581, 583; ¹H NMR (300 MHz, CDCl₃) δ 8.69 (bs, 1H), 8.04 (t, J=2.1 Hz, 1H), 7.96 (d, J=8.3 Hz, 1H), 7.89 (d, J=8.0 Hz, 1H), 7.62-7.05 (m, 2H), 7.47-7.38 (m, 2H), 6.65 (bs, 1H), 6.46 (d, J=2.8 Hz, 1H), 6.40 (dd, J=1.7, 18.2 Hz, 1H), 5.82 (d, J=10.5 Hz, 1H), 4.61-4.48 (m, 1H), 4.46-4.34 (m, 1H), 4.16-3.29 (m, 4H), 3.25-2.88 (m, 5H), 2.63 (bs, 1H), 2.49 (s, 3H), 2.39-2.18 (m, 1H), 2.10-1.68 (m, 5H).

### Example 2

### 2-((S)-4-(7-(8-Chloronaphthalen-1-yl)-2-(((S)-1-methylpyrrolidin-2-yl)methoxy)-1,5-naphthyridin-4-yl)-1-(2-fluoroacryloyl)piperazin-2-yl)acetonitrile (1q)

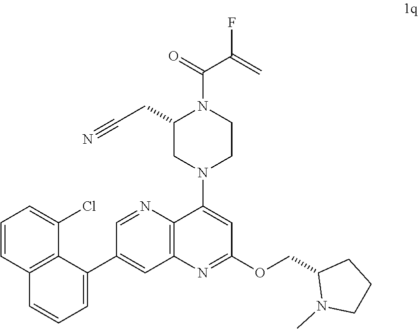

### Example 2 (1q) was Prepared as Shown Below in Scheme 3

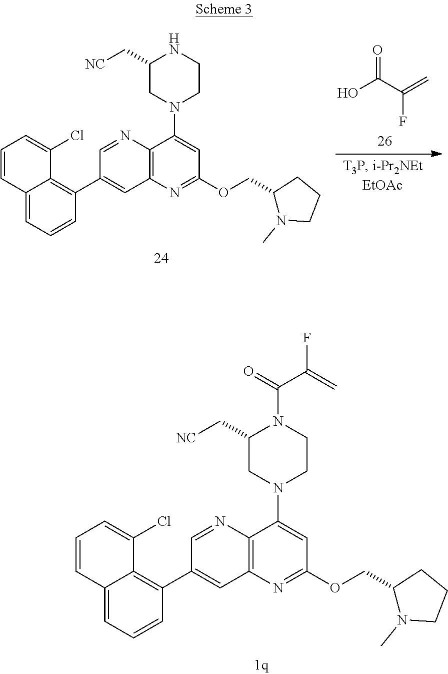

### 2-((S)-4-(7-(8-Chloronaphthalen-1-yl)-2-(((S)-1-methylpyrrolidin-2-yl)methoxy)-1,5-naphthyridin-4-yl)-1-(2-fluoroacryloyl)piperazin-2-yl)acetonitrile (1q)

1-Propanephosphonic anhydride solution (T₃P, 0.2 mL, 0.275 mmol, 50% in EtOAc) was added to a solution of 2-((S)-4-(7-(8-chloronaphthalen-1-yl)-2-(((S)-1-methylpyrrolidin-2-yl)methoxy)-1,5-naphthyridin-4-yl)piperazin-2-yl)acetonitrile (24; 29 mg, 0.055 mmol), 2-fluoroprop-2-enoic acid (26; CAS #430-99-9; 14 mg, 0.154 mmol) and diisopropylethylamine (0.1 mL, 0.55 mmol) in EtOAc (4 mL) under a N₂ atmosphere and the mixture stirred at RT. After 40 minutes, the reaction mixture was diluted with EtOAc and washed with sat. aq. NaHCO₃ (3×) and sat. aq. NaCl (2×). The organic layer was subsequently dried (MgSO₄), filtered, and concentrated in vacuo. The resulting crude product was purified by silica gel column chromatography eluting with a gradient of 10% to 100% EtOAc containing 1% Et₃N (v/v) in DCM to afford 17 mg (51%) of 2-((S)-4-(7-(8-chloronaphthalen-1-yl)-2-(((S)-1-methylpyrrolidin-2-yl)methoxy)-1,5-naphthyridin-4-yl)-1-(2-fluoroacryloyl)piperazin-2-yl)acetonitrile (1q) as an off-white solid: HPLC-MS (ES⁺) m/z [M+H⁺]=599, 601; ¹H NMR (300 MHz, CDCl₃) δ 8.69 (t, J=2.2 Hz, 1H), 8.04 (t, J=2.1 Hz, 1H), 7.96 (d, J=8.5 Hz, 1H), 7.89 (d, J=8.0 Hz, 1H), 7.59-7.50 (m, 2H), 7.47-7.37 (m, 2H), 6.46 (d, J=2.7 Hz, 1H), 5.41 (d, J=47.8 Hz, 1H), 5.24 (dd, J=3.9, 13.2 Hz, 1H), 4.60-4.47 (m, 1H), 4.45-4.33 (m, 1H), 3.87-3.72 (m, 1H), 3.50 (br s, 2H), 3.23-2.92 (m, 5H), 2.70-2.56 (m, 2H), 2.49 (s, 3H), 2.37-2.18 (m, 1H), 2.11-1.68 (m, 5H).

### Example 3

### 1-(8-(8-((1R,5S)-3,8-Diazabicyclo[3.2.1]octan-3-yl)-6-(((2R,7aS)-2-fluorotetrahydro-1H-pyrrolizin-7a(5H)-yl)methoxy)-1,5-naphthyridin-3-yl)-2-fluoro-6-hydroxynaphthalen-1-yl)ethan-1-one (2aw)

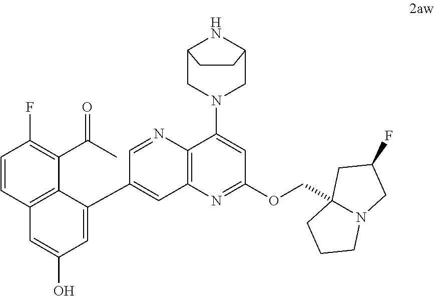 [SMILES: CC(=O)c1c(F)ccc2cc(O)cc(-c3cnc4c(N5CC6CCC(C5)N6)cc(OC[C@@]56CCCN5C[C@H](F)C6)nc4c3)c12]

### Example 3 (2aw) was Prepared as Shown Below in Scheme 4

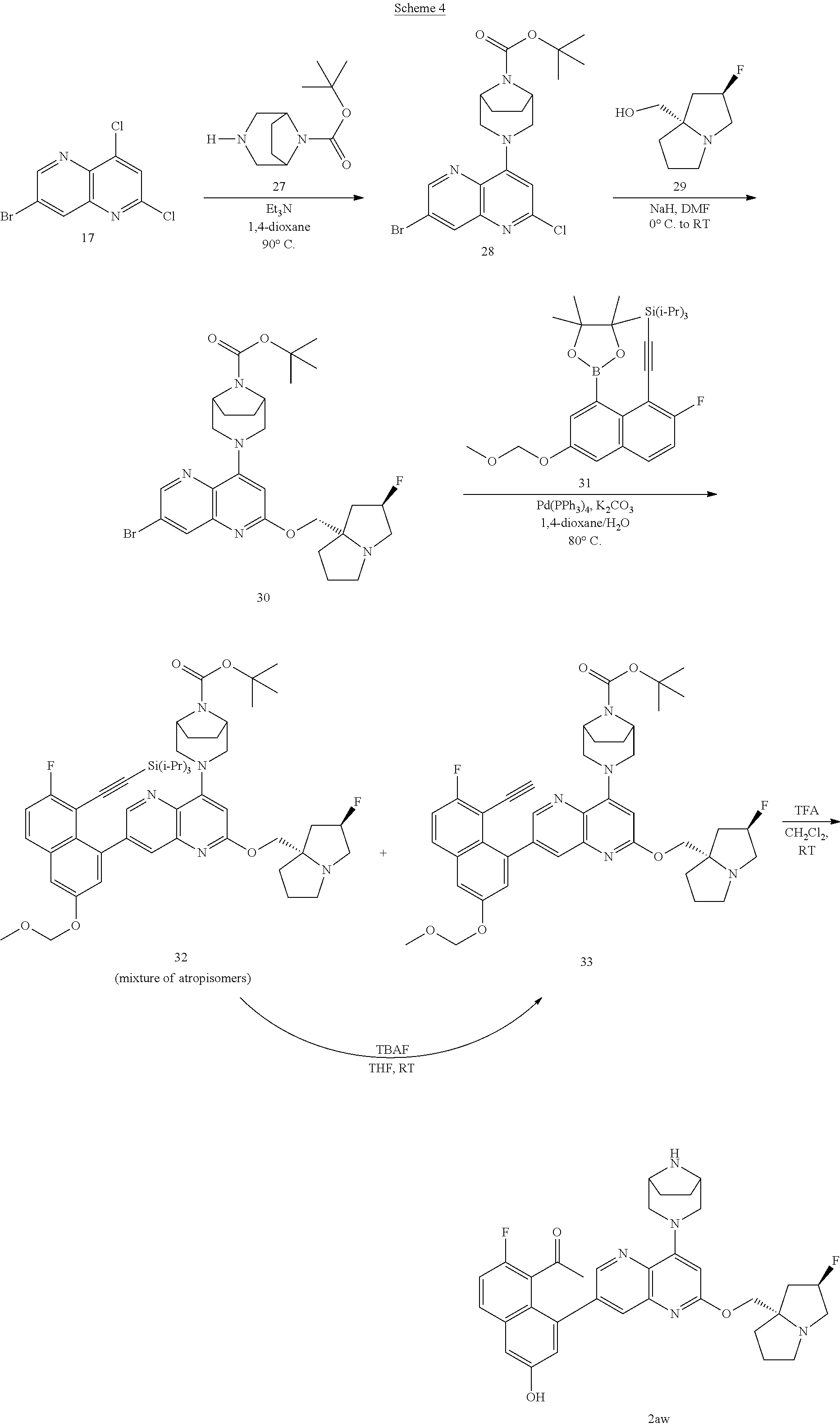

tert-Butyl (1R,5S)-3-(7-bromo-2-chloro-1,5-naphthyridin-4-yl)-3,8-diazabicyclo[3.2.1]octane-8-carboxylate (28). Triethylamine (1.80 mL, 12.5 mmol) was added to a suspension of 7-bromo-2,4-dichloro-1,5-naphthyridine (17; 1.15 g, 4.17 mmol) and tert-butyl 3,8-diazabicyclo[3.2.1]octane-8-carboxylate (27; CAS #149771-44-8; 0.97 g, 4.59 mmol) in anhydrous 1,4-dioxane (14 mL) and the mixture heated at 90° C. under a N₂ atmosphere. After 16 h, the mixture was cooled to RT, diluted with EtOAc, washed with satd. aq. NaCl (3×), dried (MgSO₄), filtered and concentrated in vacuo. The crude product was purified by silica gel column chromatography eluting with a gradient of 1% to 25% EtOAc in hexane to afford 520 mg (28%) of tert-butyl (1R,5S)-3-(7-bromo-2-chloro-1,5-naphthyridin-4-yl)-3,8-diazabicyclo[3.2.1]octane-8-carboxylate (28) as a white solid: HPLC-MS (ES⁺) m/z [M+H⁺]=453, 455, 457; ¹H NMR (300 MHz, CDCl₃) δ 8.75 (dd, J=1.1, 2.2 Hz, 1H), 8.18 (dd, J=1.1, 2.2 Hz, 1H), 6.71 (s, 1H), 4.39 (br s, 4H), 3.23 (br d, J=9.1 Hz, 1H), 2.14-1.94 (m, 5H), 1.50 (s, 9H).

tert-Butyl (1R,5S)-3-(7-bromo-2-(((2R,7aS)-2-fluorotetrahydro-1H-pyrrolizin-7a(5H)-yl)methoxy)-1,5-naphthyridin-4-yl)-3,8-diazabicyclo[3.2.1]octane-8-carboxylate (30). Sodium hydride (38 mg, 0.953 mmol, 60% mineral oil dispersion) was added to a solution of (2R,7aS)-2-fluorotetrahydro-1H-pyrrolizin-7a(5H)-yl)methanol (29; CAS #2097518-76-6; 151 mg, 0.953 mmol) in anhydrous DMF (6 mL) while cooling at 0° C. under a N₂ atmosphere. After 35 minutes, tert-butyl (1R,5S)-3-(7-bromo-2-chloro-1,5-naphthyridin-4-yl)-3,8-diazabicyclo[3.2.1]octane-8-carboxylate (28; 287 mg, 0.635 mmol) was added in one portion and the mixture warmed to RT. After 16 h, the mixture was diluted with EtOAc, washed with satd. aq. NaCl (4×), dried (MgSO₄), filtered and concentrated in vacuo. The crude product was purified by silica gel column chromatography eluting with a gradient of 0% to 10% MeOH in DCM to afford 170 mg (46%) of tert-butyl (1R,5S)-3-(7-bromo-2-(((2R,7aS)-2-fluorotetrahydro-1H-pyrrolizin-7a(5H)-yl)methoxy)-1,5-naphthyridin-4-yl)-3,8-diazabicyclo[3.2.1]octane-8-carboxylate (30) as a white foamy solid: HPLC-MS (ES⁺) m/z [M+H⁺]=576, 578; ¹H NMR (300 MHz, CDCl₃) δ 8.60 (d, J=2.2 Hz, 1H), 8.16 (d, J=2.2 Hz, 1H), 6.25 (s, 1H), 5.27 (d, J=54.1 Hz, 1H), 4.35 (br s, 2H), 4.25 (d, J=10.5 Hz, 1H), 4.13 (d, J=10.5 Hz, 1H), 3.34-3.22 (m, 2H), 3.19-2.93 (m, 4H), 2.24-1.80 (m, 10H), 1.68 (br s, 2H), 1.49 (s, 9H).

tert-Butyl (1R,5S)-3-(7-(7-fluoro-3-(methoxymethoxy)-8-((triisopropylsilyl)ethynyl)-naphthalen-1-yl)-2-(((2R,7aS)-2-fluorotetrahydro-1H-pyrrolizin-7a(5H)-yl)methoxy)-1,5-naphthyridin-4-yl)-3,8-diazabicyclo[3.2.1]octane-8-carboxylate (32). tert-Butyl (1R,5S)-3-(7-bromo-2-(((2R,7aS)-2-fluorotetrahydro-1H-pyrrolizin-7a(5H)-yl)methoxy)-1,5-naphthyridin-4-yl)-3,8-diazabicyclo[3.2.1]octane-8-carboxylate (30; 160 mg, 0.278 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (31; CAS #2621932-37-2; 168 mg, 0.328 mmol) and K₂CO₃ (156 mg, 1.14 mmol) were combined in 1,4-dioxane (3 mL) and water (0.5 mL) and the mixture was degassed by sparging with N₂ while stirring for 30 minutes. Tetrakis(triphenylphosphine)-palladium (0) (33.3 mg, 0.028 mmol) was added and the reaction mixture degassed by sparging with N₂ while stirring for an additional 20 minutes. The reaction mixture was heated at 80° C. with stirring under a N₂ atmosphere for 16 h, cooled to RT, diluted with EtOAc and filtered through Celite. The filtrate was washed with satd. aq. NaCl (2×), dried (MgSO₄), filtered and concentrated in vacuo. The crude product was a mixture of 32 and 33 that was purified by silica gel column chromatography eluting with a gradient of 30% to 100% EtOAc in hexane to afford 83 mg (34%) of tert-butyl (1R,5S)-3-(7-(7-fluoro-3-(methoxymethoxy)-8-((triisopropylsilyl)ethynyl)-naphthalen-1-yl)-2-(((2R,7aS)-2-fluorotetrahydro-1H-pyrrolizin-7a(5H)-yl)methoxy)-1,5-naphthyridin-4-yl)-3,8-diazabicyclo[3.2.1]octane-8-carboxylate (32) as an off-white solid mixture of atropisomers (HPLC-MS (ES⁺) m/z [M+H⁺]=882) and 67 mg (33%) of 33 as a yellow-orange foamy solid (HPLC-MS (ES⁺) m/z [M+H⁺]=726).

tert-Butyl (1R,5S)-3-(7-(8-ethynyl-7-fluoro-3-(methoxymethoxy) naphthalen-1-yl)-2-(((2R,7aS)-2-fluorotetrahydro-1H-pyrrolizin-7a(5H)-yl)methoxy)-1,5-naphthyridin-4-yl)-3,8-diazabicyclo[3.2.1]octane-8-carboxylate (33). Tetrabutylammonium fluoride (0.1 mL, 0.10 mmol, 1 M in THF) was added to a solution of tert-butyl (1R,5S)-3-(7-(7-fluoro-3-(methoxy-methoxy)-8-((triisopropylsilyl)ethynyl)naphthalen-1-yl)-2-(((2R,7aS)-2-fluorotetrahydro-1H-pyrrolizin-7a(5H)-yl)methoxy)-1,5-naphthyridin-4-yl)-3,8-diazabicyclo[3.2.1]octane-8-carboxylate (32; 65 mg, 0.074 mmol) in anhydrous THF (1 mL) and the mixture stirred at RT. After 1 h, the mixture was diluted with EtOAc, washed with satd. aq. NaCl (3×), dried (MgSO₄), filtered, and concentrated in vacuo to afford 53 mg of tert-butyl (1R,5S)-3-(7-(8-ethynyl-7-fluoro-3-(methoxymethoxy) naphthalen-1-yl)-2-(((2R,7aS)-2-fluorotetrahydro-1H-pyrrolizin-7a(5H)-yl)methoxy)-1,5-naphthyridin-4-yl)-3,8-diazabicyclo[3.2.1]octane-8-carboxylate (33) as a yellow-orange foamy solid: HPLC-MS (ES⁺) m/z [M+H⁺]=726; ¹H NMR (300 MHz, CDCl₃) δ 9.04 (d, J=2.2 Hz, 1H), 8.35 (d, J=2.2 Hz, 1H), 7.88 (br d, J=1.0 Hz, 1H), 7.78 (dd, J=4.1, 4.7 Hz, 1H), 7.29 (d, J=9.1 Hz, 1H), 6.29 (s, 1H), 5.35 (s, 2H), 5.30 (d, J=53.3 Hz, 1H), 4.41 (br s, 2H), 4.34 (d, J=10.5 Hz, 1H), 4.20 (d, J=10.5 Hz, 1H), 3.56 (s, 3H), 3.37-2.94 (m, 8H), 2.29-1.86 (m, 10H), 1.63 (br s, 2H), 1.50 (s, 9H).

1-(8-(8-((1R,5S)-3,8-diazabicyclo[3.2.1]octan-3-yl)-6-(((2R,7aS)-2-fluorotetrahydro-1H-pyrrolizin-7a(5H)-yl)methoxy)-1,5-naphthyridin-3-yl)-2-fluoro-6-hydroxynaphthalen-1-yl)ethan-1-one (2aw). Trifluoroacetic acid (1 mL) was added dropwise to a solution of tert-butyl (1R,5S)-3-(7-(8-ethynyl-7-fluoro-3-(methoxymethoxy) naphthalen-1-yl)-2-(((2R,7aS)-2-fluorotetrahydro-1H-pyrrolizin-7a(5H)-yl)methoxy)-1,5-naphthyridin-4-yl)-3,8-diazabicyclo[3.2.1]octane-8-carboxylate (33; 129 mg, 0.178 mmol) in dichloromethane (3 mL) and the mixture stirred at RT under a N₂ atmosphere. After 1 h, the mixture was diluted with DCM and slowly transferred by pipette into aqueous NH₄OH solution (25 mL) and stirred at RT. The phases were separated and the aqueous layer was extracted with EtOAc (2×) and the combined organic layers were dried (MgSO₄), filtered and concentrated in vacuo. The crude product was purified by silica gel column chromatography eluting with a gradient of 0% to 20% MeOH in DCM containing 5% NH₄OH (v/v)) to afford 16 mg (15%) of 1-(8-(8-((1R,5S)-3,8-diazabicyclo[3.2.1]octan-3-yl)-6-(((2R,7aS)-2-fluorotetrahydro-1H-pyrrolizin-7a(5H)-yl)methoxy)-1,5-naphthyridin-3-yl)-2-fluoro-6-hydroxynaphthalen-1-yl)ethan-1-one (2aw) as a tan solid: HPLC-MS (ES⁺) m/z [M+H⁺]=600; ¹H NMR (300 MHz, DMSO-d₆) δ 10.92 (br s, 1H), 10.26 (s, 1H), 9.29 (br s, 1H), 9.11 (br s, 1H), 8.62 (d, J=2.0 Hz, 1H), 8.02 (dd, J=4.6, 6.0 Hz, 1H), 7.91 (d, J=2.0 Hz, 1H), 7.46 (t, J=9.3 Hz, 1H), 7.40 (d, J=2.3 Hz, 1H), 7.13 (d, J=2.0 Hz, 1H), 6.62 (s, 1H), 5.59 (d, J=52.4 Hz, 1H), 4.71-4.45 (m, 3H), 4.36 (d, J=12.3 Hz, 1H), 4.25 (br s, 2H), 4.07-3.65 (m, 4H), 2.37-2.01 (m, 10H), 1.99 (s, 3H).

### Example 4

### 4-(8-((1R,5S)-3,8-Diazabicyclo[3.2.1]octan-3-yl)-6-((tetrahydro-1H-pyrrolizin-7a(5H)-yl)methoxy)-1,5-naphthyridin-3-yl)naphthalen-2-ol (2a)

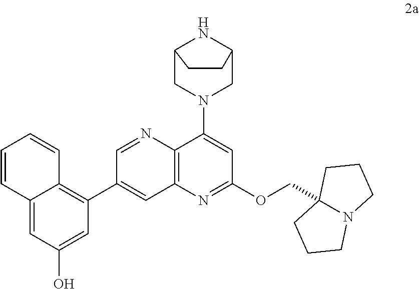

### Example 4 (2a) was Prepared as Shown Below in Scheme 5

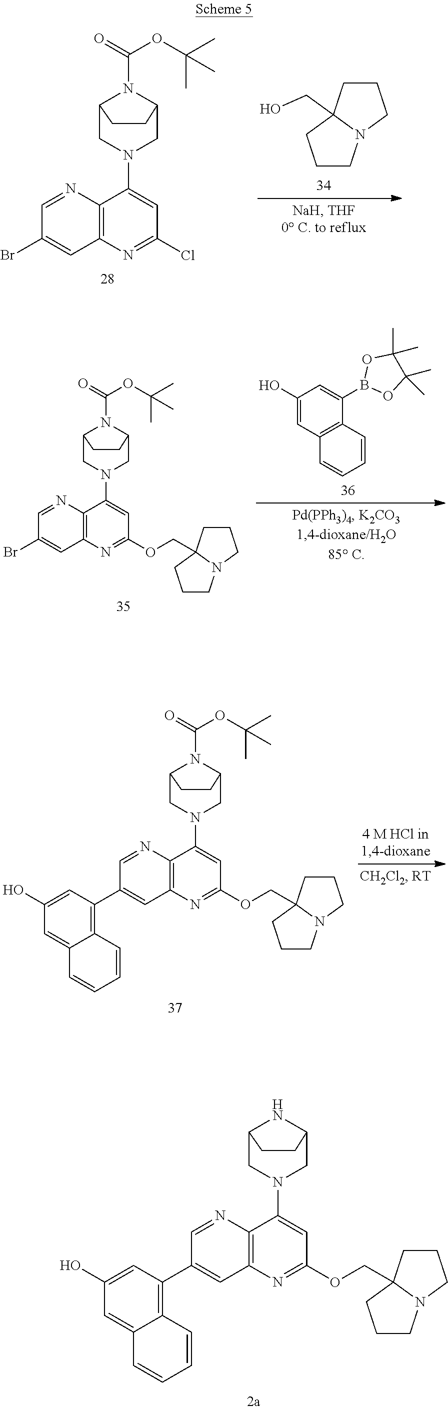 [SMILES: CC(C)(C)OC(=O)N1C2CCC1CN(c1cc(Cl)nc3cc(Br)cnc13)C2 ; OCC12CCCN1CCC2 ; CC(C)(C)OC(=O)N1C2CCC1CN(c1cc(OCC34CCCN3CCC4)nc3cc(Br)cnc13)C2 ; CC1(C)OB(c2cc(O)cc3ccccc23)OC1(C)C ; CC(C)(C)OC(=O)N1C2CCC1CN(c1cc(OCC34CCCN3CCC4)nc3cc(-c4cc(O)cc5ccccc45)cnc13)C2 ; Oc1cc(-c2cnc3c(N4CC5CCC(C4)N5)cc(OCC45CCCN4CCC5)nc3c2)c2ccccc2c1]

tert-Butyl (1R,5S)-3-(7-bromo-2-((tetrahydro-1H-pyrrolizin-7a(5H)-yl)methoxy)-1,5-naphthyridin-4-yl)-3,8-diazabicyclo[3.2.1]octane-8-carboxylate (35). Sodium hydride (70 mg, 1.72 mmol, 60% w/w dispersion in mineral oil) was added to a solution of hexahydro-1H-pyrrolizin-7a-ylmethanol (34; CAS #78449-72-6; 242 mg, 1.72 mmol) in anhydrous THF (8 mL) at 0° C. under a N₂ atmosphere. After 30 minutes, tert-butyl (1R,5S)-3-(7-bromo-2-chloro-1,5-naphthyridin-4-yl)-3,8-diazabicyclo[3.2.1]octane-8-carboxylate (28; 520 mg, 1.15 mmol) was added in one portion and the mixture heated at reflux. After 16 h, the mixture was cooled to RT, diluted with EtOAc, washed with satd. aq. NaCl (3×), dried (MgSO₄), filtered and concentrated in vacuo. The crude product was purified by silica gel column chromatography eluting with gradient of 0% to 10% MeOH in DCM containing 10% NH₄OH (v/v) to afford 300 mg (47%) of tert-butyl (1R,5S)-3-(7-bromo-2-((tetrahydro-1H-pyrrolizin-7a(5H)-yl)methoxy)-1,5-naphthyridin-4-yl)-3,8-diazabicyclo[3.2.1]octane-8-carboxylate (35) as a white solid: HPLC-MS (ES⁺) m/z [M+H⁺]=558, 560; ¹H NMR (300 MHz, CDCl₃) δ 8.59 (d, J=2.2 Hz, 1H), 8.17 (d, J=2.2 Hz, 1H), 6.28 (s, 1H), 4.35 (br s, 2H), 4.22 (br s, 2H), 4.19 (s, 2H), 3.18-2.97 (m, 4H), 2.73-2.59 (m, 2H), 2.20-2.08 (m, 2H), 2.03-1.76 (m, 8H), 1.68-1.56 (m, 2H), 1.48 (s, 9H).

tert-Butyl (1R,5S)-3-(7-(3-hydroxynaphthalen-1-yl)-2-((tetrahydro-1H-pyrrolizin-7a(5/I)-yl)methoxy)-1,5-naphthyridin-4-yl)-3,8-diazabicyclo[3.2.1]octane-8-carboxylate (37). A mixture of tert-butyl (1R,5S)-3-(7-bromo-2-((tetrahydro-1H-pyrrolizin-7a(5H)-yl)methoxy)-1,5-naphthyridin-4-yl)-3,8-diazabicyclo[3.2.1]octane-8-carboxylate (35; 290 mg, 0.520 mmol), 4-(4,4,5,5-tetramethyl-1,3,2-dioxaborolan-2-yl)naphthalen-2-ol (36; CAS #2043962-01-0; 281 mg, 1.04 mmol), K₂CO₃ (293 mg, 2.13 mmol) in 1,4-dioxane (5 mL) and water (1 mL) was degassed by sparging with N₂ for 30 minutes. Tetrakis(triphenylphosphine)-palladium (0) (60 mg, 0.052 mmol) was added and the reaction mixture degassed by sparging with N₂ for an additional 20 minutes. After sparging was complete, the reaction mixture was heated at 85° C. with stirring under a N₂ atmosphere for 16 h. The reaction mixture was cooled to RT, diluted with EtOAc and filtered through Celite. The organic layer was washed with satd. aq. NaCl (3×), dried (MgSO₄), filtered and concentrated in vacuo. The crude product was purified by silica gel column chromatography eluting with a gradient of 0% to 10% MeOH in DCM containing 10% NH₄OH (v/v) to afford 184 mg (57%) of tert-butyl (1R,5S)-3-(7-(3-hydroxynaphthalen-1-yl)-2-((tetrahydro-1H-pyrrolizin-7a(5H)-yl)methoxy)-1,5-naphthyridin-4-yl)-3,8-diazabicyclo[3.2.1]octane-8-carboxylate (37) as a tan solid: HPLC-MS (ES⁺) m/z [M+H⁺]=622; ¹H NMR (300 MHz, DMSO-d₆) δ 9.98 (br s, 1H), 8.72 (d, J=2.2 Hz, 1H), 8.02 (d, J=2.2 Hz, 1H), 7.81 (br d, J=8.1 Hz, 1H), 7.64 (br d, J=8.4 Hz, 1H), 7.45 (br t, J=5.0 Hz, 1H), 7.31-7.22 (m, 2H), 7.14 (d, J=2.4 Hz, 1H), 6.38 (s, 1H), 4.35 (br d, J=11.1 Hz, 2H), 4.27 (br s, 2H), 4.06 (s, 2H), 3.07 (br d, J=11.0 Hz, 2H), 3.00-2.86 (m, 2H), 2.60-2.50 (m, 1H), 2.08 (br d, J=7.3 Hz, 2H), 1.97-1.68 (m, 9H), 1.63-1.49 (m, 2H), 1.44 (s, 9H).

4-(8-((1R,5S)-3,8-diazabicyclo[3.2.1]octan-3-yl)-6-((tetrahydro-1H-pyrrolizin-7a(5H)-yl)methoxy)-1,5-naphthyridin-3-yl)naphthalen-2-ol (2a). A solution of tert-butyl (1R,5S)-3-(7-(3-hydroxynaphthalen-1-yl)-2-((tetrahydro-1H-pyrrolizin-7a(5H)-yl)methoxy)-1,5-naphthyridin-4-yl)-3,8-diazabicyclo[3.2.1]octane-8-carboxylate (37; 172 mg, 0.277 mmol) in DCM (3 mL) was treated slowly dropwise with 4 M HCl in 1,4-dioxane (3 mL) and the resulting orange suspension was stirred at RT under a N₂ atmosphere. After 2 h, the reaction was allowed to stand at RT. After 72 h, MeOH was added to the mixture, diluted with DCM, and the resulting solution slowly transferred by pipette into NH₄OH (aq) and stirred at RT. The phases were separated and the aqueous layer extracted once with EtOAc and once with DCM. The organic layers were combined and dried (MgSO₄), filtered and concentrated in vacuo. The resulting crude product was purified by silica gel column chromatography eluting with a gradient of 0% to 15% MeOH in DCM containing 10% NH₄OH (v/v) to afford 102 mg (71%) of 4-(8-((1R,5S)-3,8-diazabicyclo[3.2.1]octan-3-yl)-6-((tetrahydro-1H-pyrrolizin-7a(5H)-yl)methoxy)-1,5-naphthyridin-3-yl)naphthalen-2-ol (2a) as a tan solid: HPLC-MS (ES⁺) m/z [M+H⁺]=522; ¹H NMR (300 MHz, DMSO-d₆) δ 10.0 (br s, 1H), 8.70 (d, J=2.2 Hz, 1H), 8.00 (d, J=2.2 Hz, 1H), 7.81 (br d, J=8.1 Hz, 1H), 7.64 (br d, J=8.4 Hz, 1H), 7.45 (br t, J=5.0 Hz, 1H), 7.32-7.21 (m, 2H), 7.14 (d, J=2.4 Hz, 1H), 6.26 (s, 1H), 4.29 (br d, J=9.5 Hz, 2H), 4.05 (s, 2H), 3.51 (br s, 2H), 3.32 (br s, 2H), 3.01 (br d, J=10.7 Hz, 2H), 2.97-2.87 (m, 2H), 2.60-2.50 (m, 1H), 2.04-1.64 (m, 10H), 1.62-1.48 (m, 2H).

### Example 5

### (S)-2-(1-acryloyl-4-(7-(8-chloronaphthalen-1-yl)-2-((tetrahydro-1H-pyrrolizin-7a(5H)-yl)methoxy)-1,5-naphthyridin-4-yl)piperazin-2-yl)acetonitrile (1ae)

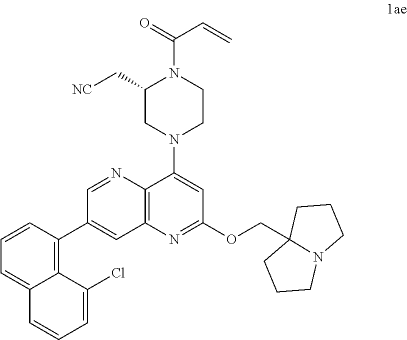 [SMILES: C=CC(=O)N1CCN(c2cc(OCC34CCCN3CCC4)nc3cc(-c4cccc5cccc(Cl)c45)cnc23)C[C@@H]1CC#N]

### Example 5 (1ae) was Prepared as Shown Below in Scheme 6

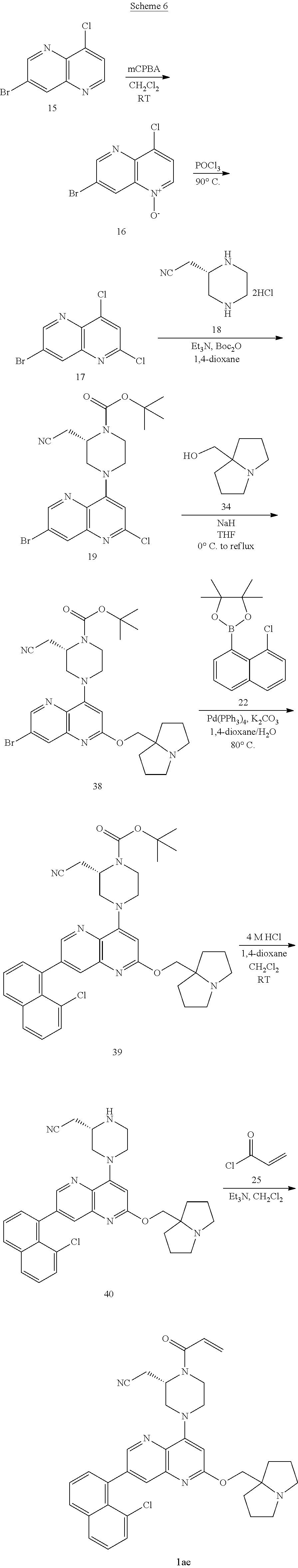

tert-butyl(S)-4-(7-bromo-2-((tetrahydro-1H-pyrrolizin-7a(5H)-yl)methoxy)-1,5-naphthyridin-4-yl)-2-(cyanomethyl)piperazine-1-carboxylate (38). Sodium hydride (142 mg, 3.54 mmol, 60% w/w dispersion in mineral oil) was added to a solution of hexahydro-1H-pyrrolizin-7a-ylmethanol (34; CAS #78449-72-6; 500 mg, 3.54 mmol) in anhydrous THF (20 mL) at 0° C. under a N₂ atmosphere. After 30 minutes, tert-butyl(S)-4-(7-bromo-2-chloro-1,5-naphthyridin-4-yl)-2-(cyanomethyl)piperazine-1-carboxylate (19; 1.10 g, 2.36 mmol) was added in one portion and the mixture heated at reflux. After 16 h, the mixture was cooled to RT, diluted with EtOAc, washed with satd. aq. NaCl (3×), dried (MgSO₄), filtered, and concentrated in vacuo. The crude product was purified by silica gel column chromatography eluting with gradient of 0% to 6% MeOH in DCM containing 10% NH₄OH (v/v) to afford 734 mg (54%) of tert-butyl(S)-4-(7-bromo-2-((tetrahydro-1H-pyrrolizin-7a(5H)-yl)methoxy)-1,5-naphthyridin-4-yl)-2-(cyanomethyl)piperazine-1-carboxylate (38) as a white foam: HPLC-MS (ES⁺) m/z [M+H⁺]=571, 573; ¹H NMR (300 MHz, CDCl₃) δ 8.71 (d, J=2.0 Hz, 1H), 8.20 (d, J=2.2 Hz, 1H), 6.40 (s, 1H), 4.72 (d, J=12.5 Hz, 1H), 4.61 (br s, 1H), 4.21 (d, J=3.6 Hz, 2H), 4.13 (br s, 1H), 3.62 (d, J=4.3 Hz, 1H), 3.43-3.18 (m, 2H), 3.17-3.05 (m, 2H), 3.04-2.91 (m, 2H), 2.86 (dd, J=5.7, 16.4 Hz, 1H), 2.74-2.60 (m, 2H), 2.08-1.77 (m, 6H), 1.71-1.57 (m, 2H), 1.53 (s, 9H).

tert-butyl(S)-4-(7-(8-chloronaphthalen-1-yl)-2-((tetrahydro-1H-pyrrolizin-7a(5H)-yl)methoxy)-1,5-naphthyridin-4-yl)-2-(cyanomethyl)piperazine-1-carboxylate (39). A mixture of tert-butyl(S)-4-(7-bromo-2-((tetrahydro-1H-pyrrolizin-7a(5H)-yl)methoxy)-1,5-naphthyridin-4-yl)-2-(cyanomethyl)piperazine-1-carboxylate (38; 722 mg, 1.27 mmol), 2-(8-chloronaphthalen-1-yl)-4,4,5,5-tetramethyl-1,3,2-dioxaborolane (22; CAS #: 2454397-84-1; 731 mg, 2.54 mmol), and K₂CO₃ (718 mg, 5.21 mmol) in dioxane (12 mL) and water (2.4 mL) was degassed by sparging with N₂ with stirring for 30 minutes.

Tetrakis(triphenylphosphine) palladium (0) (147 mg, 0.127 mmol) was added and the reaction mixture degassed by sparging with N₂ with stirring for an additional 20 minutes. The reaction mixture was heated at 80° C. with stirring under a N₂ atmosphere for 16 h. The reaction mixture was cooled to RT, diluted with EtOAc, and filtered through Celite. The filtrate was washed with satd. aq. NaCl (3×), dried (MgSO₄), filtered, and concentrated in vacuo. The crude product was purified by silica gel column chromatography (eluting with a gradient of 0% to 6% MeOH in DCM containing 10% NH₄OH (v/v) to afford 362 mg (44%) of tert-butyl(S)-4-(7-(8-chloronaphthalen-1-yl)-2-(((S)-1-methylpyrrolidin-2-yl)methoxy)-1,5-naphthyridin-4-yl)-2-(cyanomethyl)piperazine-1-carboxylate (39) as a white foam: HPLC-MS (ES⁺) m/z [M+H⁺]=653, 655; ¹H NMR (300 MHz, CDCl₃) δ 8.66 (t, J=2.2 Hz, 1H), 8.01 (t, J=2.0 Hz, 1H), 7.95 (d, J=8.1 Hz, 1H), 7.88 (d, J=8.1 Hz, 1H), 7.61-7.49 (m, 2H), 7.47-7.37 (m, 2H), 6.44 (d, J=3.2 Hz, 1H), 4.86 (dd, J=12.5, 40.0 Hz, 1H), 4.68 (br s, 1H), 4.36-3.96 (m, 2H), 3.87-3.56 (m, 1H), 3.49-3.20 (m, 2H), 3.18-2.83 (m, 6H), 2.77-2.54 (m, 2H), 2.10-1.77 (m, 6H), 1.71-1.57 (m, 2H), 1.53 (s, 9H).

(S)-2-(4-(7-(8-chloronaphthalen-1-yl)-2-((tetrahydro-1H-pyrrolizin-7a(5H)-yl)methoxy)-1,5-naphthyridin-4-yl)piperazin-2-yl)acetonitrile (40). A solution of tert-butyl(S)-4-(7-(8-chloronaphthalen-1-yl)-2-(((S)-1-methylpyrrolidin-2-yl)methoxy)-1,5-naphthyridin-4-yl)-2-(cyanomethyl)piperazine-1-carboxylate (39; 349 mg, 0.534 mmol) in CH₂Cl₂ (10 mL) was treated with a solution of 4 M HCl/dioxane (3.3 mL) slowly dropwise resulting in the formation of an orange gooey solid and the mixture stirred at RT. After 3 h, the solid was sampled and determined to be product by LC/MS. The DCM layer was decanted off and the gooey solid dissolved in MeOH. The solution was diluted with sat. aq. NaHCO₃, extracted with DCM (5×), dried (MgSO₄), filtered, and concentrated in vacuo. The crude product was purified by silica gel column chromatography eluting with gradient of 0% to 10% MeOH in DCM containing 10% NH₄OH (v/v) to afford 191 mg (65%) of(S)-2-(4-(7-(8-chloronaphthalen-1-yl)-2-((tetrahydro-1H-pyrrolizin-7a(5H)-yl)methoxy)-1,5-naphthyridin-4-yl)piperazin-2-yl)acetonitrile (40) as a white foam: HPLC-MS (ES⁺) m/z [M+H⁺]=554, 556; ¹H NMR (300 MHz, CDCl₃) δ 8.63 (d, J=2.1 Hz, 1H), 8.00 (d, J=2.0 Hz, 1H), 7.96 (d, J=8.1 Hz, 1H), 7.88 (d, J=8.1 Hz, 1H), 7.60-7.49 (m, 2H), 7.47-7.36 (m, 2H), 6.45 (s, 1H), 4.23 (d, J=4.8 Hz, 2H), 3.95 (dd, J=11.4, 33.2 Hz, 1H), 3.60-3.39 (m, 1H), 3.34-2.96 (m, 6H), 2.76-2.50 (m, 4H), 2.12-1.73 (m, 8H), 1.71-1.50 (m, 2H).

(S)-2-(1-acryloyl-4-(7-(8-chloronaphthalen-1-yl)-2-((tetrahydro-1H-pyrrolizin-7a(5H)-yl)methoxy)-1,5-naphthyridin-4-yl)piperazin-2-yl)acetonitrile (1ae). A solution of(S)-2-(4-(7-(8-chloronaphthalen-1-yl)-2-((tetrahydro-1H-pyrrolizin-7a(5H)-yl)methoxy)-1,5-naphthyridin-4-yl) piperazin-2-yl)acetonitrile (40; 83 mg, 0.15 mmol) in CH₂Cl₂ (12 mL) was treated with Et₃N (25 μL, 0.17 mmol). The mixture was cooled to 0° C., acryloyl chloride (25; CAS #814-68-6; 15 μL, 0.17 mmol) added, and the mixture stirred in the ice bath. After 1.5 h, the mixture was diluted with CH₂Cl₂, washed with H₂O (2×), dried (MgSO₄), filtered, and concentrated in vacuo. The crude product was purified by silica gel column chromatography (eluting with a gradient of 0% to 5% MeOH in DCM containing 10% NH₄OH (v/v)) to afford 45 mg (49%) of(S)-2-(1-acryloyl-4-(7-(8-chloronaphthalen-1-yl)-2-((tetrahydro-1H-pyrrolizin-7a(5H)-yl)methoxy)-1,5-naphthyridin-4-yl)piperazin-2-yl)acetonitrile (1ae) as an off-white powder: HPLC-MS (ES⁺) m/z [M+H⁺]=607, 609; ¹H NMR (300 MHz, CDCl₃) δ 8.68 (br t, J=1.9 Hz, 1H), 8.02 (t, J=1.9 Hz, 1H), 7.96 (d, J=8.2 Hz, 1H), 7.88 (d, J=8.0 Hz, 1H), 7.62-7.49 (m, 2H), 7.47-7.35 (m, 2H), 6.64 (br s, 1H), 6.45 (d, J=2.9 Hz, 1H), 6.40 (dd, J=1.4, 16.8 Hz, 1H), 5.82 (d, J=10.3 Hz, 1H), 5.36-4.42 (m, 2H), 4.36-4.14 (m, 2H), 4.12-3.26 (m, 3H), 3.24-2.81 (m, 6H), 2.75-2.52 (m, 2H), 2.10-1.52 (m, 8H).

### Example 6

### (S)-2-(4-(7-(8-chloronaphthalen-1-yl)-2-((tetrahydro-1H-pyrrolizin-7a(5H)-yl)methoxy)-1,5-naphthyridin-4-yl)-1-(2-fluoroacryloyl)piperazin-2-yl)acetonitrile (1af)

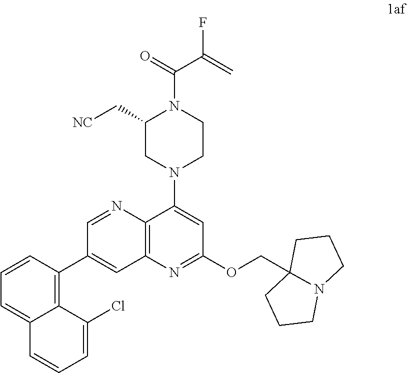 [SMILES: C=C(F)C(=O)N1CCN(c2cc(OCC34CCCN3CCC4)nc3cc(-c4cccc5cccc(Cl)c45)cnc23)C[C@@H]1CC#N]

### Example 6 (1af) was Prepared as Shown Below in Scheme 7

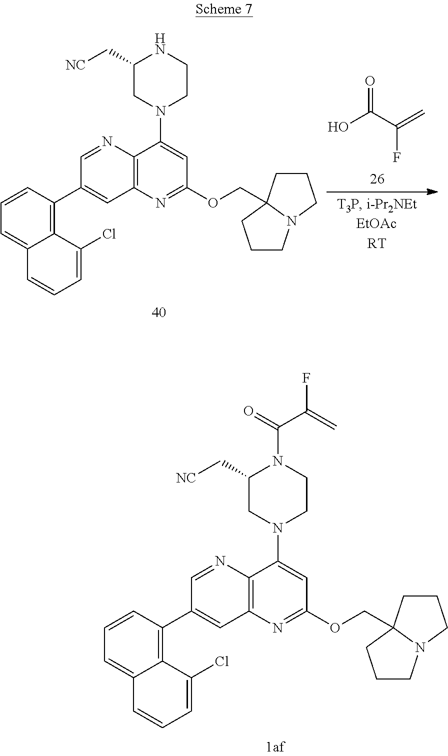

(S)-2-(4-(7-(8-chloronaphthalen-1-yl)-2-((tetrahydro-1H-pyrrolizin-7a(5H)-yl)methoxy)-1,5-naphthyridin-4-yl)-1-(2-fluoroacryloyl)piperazin-2-yl)acetonitrile (1af). 1-propanephosphonic anhydride solution (T₃P, 0.48 mL, 0.75 mmol, 50% in EtOAc) was added to a mixture of(S)-2-(4-(7-(8-chloronaphthalen-1-yl)-2-((tetrahydro-1H-pyrrolizin-7a(5H)-yl)methoxy)-1,5-naphthyridin-4-yl)piperazin-2-yl)acetonitrile (40; 83 mg, 0.15 mmol), 2-fluoroprop-2-enoic acid (26; CAS #430-99-9; 41 mg, 0.45 mmol) and diisopropylethylamine (0.27 mL, 1.50 mmol) in EtOAc (11 mL) under a N₂ atmosphere and the mixture stirred at RT. After 40 minutes, additional 1-propanephosphonic anhydride solution (T₃P, 0.3 mL) was added and the mixture stirred at RT. After 1.5 h, the reaction mixture was diluted with EtOAc, washed with 5% aq. NaHCO₃ (3×), dried (MgSO₄), filtered, and concentrated in vacuo. The crude product was purified by silica gel column chromatography (eluting with a gradient of 10% to 100% EtOAc containing 5% Et₃N (v/v)) to afford 25 mg (27%) of S)-2-(4-(7-(8-chloronaphthalen-1-yl)-2-((tetrahydro-1H-pyrrolizin-7a(5H)-yl)methoxy)-1,5-naphthyridin-4-yl)-1-(2-fluoroacryloyl)piperazin-2-yl)acetonitrile (1af) as an off-white powder: HPLC-MS (ES⁺) m/z [M+H⁺]=625, 627; ¹H NMR (300 MHz, CDCl₃) δ 8.68 (t, J=2.2 Hz, 1H), 8.02 (t, J=2.1 Hz, 1H), 7.96 (d, J=8.2 Hz, 1H), 7.88 (d, J=8.0 Hz, 1H), 7.61-7.49 (m, 2H), 7.47-7.36 (m, 2H), 6.45 (d, J=3.2 Hz, 1H), 5.41 (br d, J=48.4 Hz, 1H), 5.25 (dd, J=3.6, 16.9 Hz, 1H), 5.15-4.41 (m, 2H), 4.35-4.14 (m, 2H), 3.81 (br d, J=9.4 Hz, 1H), 3.72-3.29 (m, 2H), 3.21-2.90 (m, 5H), 2.77-2.55 (m, 2H), 2.09-1.78 (m, 7H), 1.72-1.57 (m, 2H).

### Example 7

### 2-((S)-1-acryloyl-4-(7-(8-chloronaphthalen-1-yl)-2-(((2R,7aS)-2-fluorotetrahydro-1H-pyrrolizin-7a(5H)-yl)methoxy)-1,5-naphthyridin-4-yl)piperazin-2-yl)acetonitrile (1ag)

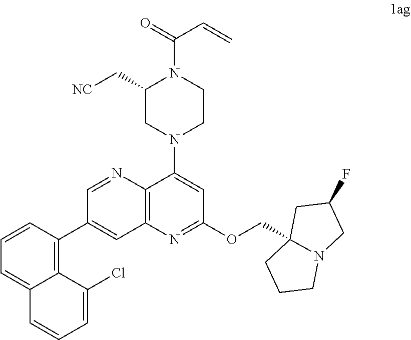 [SMILES: C=CC(=O)N1CCN(c2cc(OC[C@@]34CCCN3C[C@H](F)C4)nc3cc(-c4cccc5cccc(Cl)c45)cnc23)C[C@@H]1CC#N]

### Example 7 (1ag) was Prepared as Shown Below in Scheme 8

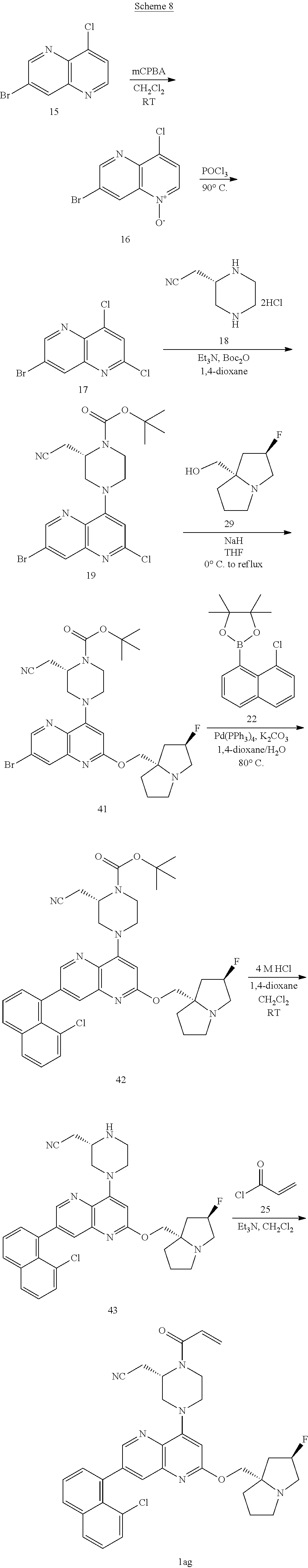

tert-butyl(S)-4-(7-bromo-2-(((2R,7aS)-2-fluorotetrahydro-1H-pyrrolizin-7a(5H)-yl)methoxy)-1,5-naphthyridin-4-yl)-2-(cyanomethyl)piperazine-1-carboxylate (41). Sodium hydride (142 mg, 3.54 mmol, 60% w/w dispersion in mineral oil) was added to a solution of (2R, 7aS)-2-fluorotetrahydro-1H-pyrrolizin-7a(5H)-yl)methanol (29; CAS #2097518-76-6; 563 mg, 3.54 mmol) in anhydrous THF (20 mL) at 0° C. under a N₂ atmosphere. After 30 minutes, tert-butyl(S)-4-(7-bromo-2-chloro-1,5-naphthyridin-4-yl)-2-(cyanomethyl)piperazine-1-carboxylate (19; 1.10 g, 2.36 mmol) was added in one portion, the cooling bath removed, and the mixture stirred at RT. After 30 min, the mixture was heated at reflux. After 16 h, the mixture was cooled to RT, diluted with EtOAc, washed with satd. aq. NaCl (3×), dried (MgSO₄), filtered, and concentrated in vacuo. The crude solid was suspended in Et₂O and stirred at RT. After 16 h, the solid was filtered, washed with Et₂O, and dried to afford 900 mg (65%) of tert-butyl(S)-4-(7-bromo-2-(((2R,7aS)-2-fluorotetrahydro-1H-pyrrolizin-7a(5H)-yl)methoxy)-1,5-naphthyridin-4-yl)-2-(cyanomethyl)piperazine-1-carboxylate (41) as an off-white solid: HPLC-MS (ES⁺) m/z [M+H⁺]=589, 591; ¹H NMR (300 MHz, CDCl₃) δ 8.72 (d, J=2.2 Hz, 1H), 8.19 (d, J=2.2 Hz, 1H), 6.38 (s, 1H), 5.28 (br d, J=54.1 Hz, 1H), 4.73 (d, J=12.7 Hz, 1H), 4.62 (br s, 1H), 4.29 (d, J=10.6 Hz, 1H), 4.14 (d, J=10.6 Hz, 2H), 3.62 (br d, J=11.3 Hz, 1H), 3.39-3.10 (m, 5H), 3.05-2.90 (m, 3H), 2.84 (dd, J=5.6, 16.4 Hz, 1H), 2.21 (br d, J=2.9 Hz, 1H), 2.14-1.79 (m, 5H), 1.52 (s, 9H).

tert-butyl(S)-4-(7-(8-chloronaphthalen-1-yl)-2-(((2R,7aS)-2-fluorotetrahydro-1H-pyrrolizin-7a(5H)-yl)methoxy)-1,5-naphthyridin-4-yl)-2-(cyanomethyl)piperazine-1-carboxylate (42). A mixture of tert-butyl(S)-4-(7-bromo-2-(((2R,7aS)-2-fluorotetrahydro-1H-pyrrolizin-7a(5H)-yl)methoxy)-1,5-naphthyridin-4-yl)-2-(cyanomethyl)piperazine-1-carboxylate (41; 800 mg, 1.36 mmol), 2-(8-chloronaphthalen-1-yl)-4,4,5,5-tetramethyl-1,3,2-dioxaborolane (22; CAS #: 2454397-84-1; 783 mg, 2.72 mmol), and K₂CO₃ (563 mg, 4.08 mmol) in dioxane (7 mL) and water (4 mL) was degassed by sparging with N₂ with stirring for 20 minutes. Tetrakis(triphenylphosphine) palladium (0) (78 mg, 0.068 mmol) was added and the reaction mixture degassed by sparging with N₂ with stirring for an additional 20 minutes. The reaction mixture was heated at 80° C. with stirring under a N₂ atmosphere for 16 h. The reaction mixture was cooled to RT, and diluted with EtOAc. The filtrate was washed with satd. aq. NaCl (3×), dried (MgSO₄), filtered, and concentrated in vacuo. The crude product was purified by silica gel column chromatography (eluting with a gradient of 0% to 5% MeOH in DCM) to afford 400 mg (44%) of tert-butyl(S)-4-(7-(8-chloronaphthalen-1-yl)-2-(((2R,7aS)-2-fluorotetrahydro-1H-pyrrolizin-7a(5H)-yl)methoxy)-1,5-naphthyridin-4-yl)-2-(cyanomethyl)piperazine-1-carboxylate (42) as a beige solid: HPLC-MS (ES⁺) m/z [M+H⁺]=671, 673; ¹H NMR (300 MHz, CDCl₃) δ 8.67 (t, J=2.4 Hz, 1H), 8.01 (dd, J=1.3, 2.0 Hz, 1H), 7.95 (d, J=8.2 Hz, 1H), 7.88 (dd, J=1.2, 8.1 Hz, 1H), 7.60-7.49 (m, 2H), 7.47-7.36 (m, 2H), 6.42 (d, J=3.7 Hz, 1H), 5.29 (br d, J=53.7 Hz, 1H), 4.86 (dd, J=12.3, 40.2 Hz, 1H), 4.68 (br s, 1H), 4.33 (dd, J=7.8, 10.6 Hz, 1H), 4.17 (t, J=9.7 Hz, 2H), 3.72 (br t, J=11.4 Hz, 1H), 3.49-3.14 (m, 5H), 3.10-2.83 (m, 4H), 2.32-1.64 (m, 6H), 1.52 (s, 9H).

2-((S)-4-(7-(8-chloronaphthalen-1-yl)-2-(((2R,7aS)-2-fluorotetrahydro-1H-pyrrolizin-7a(5H)-yl)methoxy)-1,5-naphthyridin-4-yl)piperazin-2-yl)acetonitrile (43). A solution of tert-butyl(S)-4-(7-(8-chloronaphthalen-1-yl)-2-(((2R,7aS)-2-fluorotetrahydro-1H-pyrrolizin-7a(5H)-yl)methoxy)-1,5-naphthyridin-4-yl)-2-(cyanomethyl)piperazine-1-carboxylate (42; 388 mg, 0.58 mmol) in CH₂Cl₂ (10 mL) was treated with a solution of 4 M HCl/dioxane (3.6 mL) under a N₂ atmosphere slowly dropwise resulting in the formation of a gooey solid and the mixture stirred at RT. After 2.5 h, the solid was sampled and determined to be product by LC/MS. The DCM layer was decanted off and the gooey solid dissolved in MeOH. The solution was diluted with sat. aq. NaHCO₃, extracted with DCM (3×), dried (MgSO₄), filtered, and concentrated in vacuo. The crude product was purified by silica gel column chromatography eluting with gradient of 0% to 10% MeOH in DCM containing 10% NH₄OH (v/v) to afford 198 mg (60%) of 2-((S)-4-(7-(8-chloronaphthalen-1-yl)-2-(((2R,7aS)-2-fluorotetrahydro-1H-pyrrolizin-7a(5H)-yl)methoxy)-1,5-naphthyridin-4-yl)piperazin-2-yl)acetonitrile (43) as an off-white solid: HPLC-MS (ES⁺) m/z [M+H⁺]=571, 573; ¹H NMR (300 MHz, DMSO-d₆) δ 8.58 (t, J=2.2 Hz, 1H), 8.16 (dd, J=1.1, 8.2 Hz, 1H), 8.10 (dd, J=1.2, 8.1 Hz, 1H), 7.93 (t, J=1.9 Hz, 1H), 7.73-7.62 (m, 2H), 7.61-7.50 (m, 2H), 6.42 (s, 1H), 5.29 (br d, J=54.1 Hz, 1H), 4.36-4.17 (m, 1H), 4.08 (dd, J=10.4, 26.5 Hz, 3H), 3.23-2.60 (m, 12H), 2.25-1.68 (m, 6H).

2-((S)-1-acryloyl-4-(7-(8-chloronaphthalen-1-yl)-2-(((2R,7aS)-2-fluorotetrahydro-1H-pyrrolizin-7a(5H)-yl)methoxy)-1,5-naphthyridin-4-yl)piperazin-2-yl)acetonitrile (1ag). A solution of 2-((S)-4-(7-(8-chloronaphthalen-1-yl)-2-(((2R,7aS)-2-fluorotetrahydro-1H-pyrrolizin-7a(5H)-yl)methoxy)-1,5-naphthyridin-4-yl)piperazin-2-yl)acetonitrile (43; 90 mg, 0.16 mmol) in CH₂Cl₂ (12 mL) was treated with Et₃N (25 μL, 0.17 mmol). The mixture was cooled to 0° C., acryloyl chloride (25; CAS #814-68-6; 15 μL, 0.17 mmol) was added, and the mixture stirred in the ice bath. After 1.5 h, the mixture was again cooled to 0° C. and an additional 6.8 μL of Et₃N and 3.8 μL of acryloyl chloride were added and stirred in the ice bath. After 1 h, the mixture was diluted with CH₂Cl₂, washed with H₂O (2×), dried (MgSO₄), filtered, and concentrated in vacuo. The crude product was purified by silica gel column chromatography (eluting with a gradient of 0% to 10% MeOH in DCM) to afford 73 mg (74%) of 2-((S)-1-acryloyl-4-(7-(8-chloronaphthalen-1-yl)-2-(((2R,7aS)-2-fluorotetrahydro-1H-pyrrolizin-7a(5H)-yl)methoxy)-1,5-naphthyridin-4-yl)piperazin-2-yl)acetonitrile (1ag) as an off-white solid: HPLC-MS (ES⁺) m/z [M+H⁺]=625, 627; ¹H NMR (300 MHz, CDCl₃) δ 8.69 (br t, J=2.1 Hz, 1H), 8.02 (dd, J=1.0, 2.1 Hz, 1H), 7.96 (dd, J=1.1, 8.2 Hz, 1H), 7.88 (dd, J=1.2, 8.1 Hz, 1H), 7.59-7.50 (m, 2H), 7.47-7.38 (m, 2H), 6.63 (br s, 1H), 6.46-6.33 (m, 2H), 5.82 (d, J=10.6 Hz, 1H), 5.29 (br d, J=53.2 Hz, 1H), 5.06-4.45 (m, 2H), 4.35 (dd, J=7.1, 10.4 Hz, 1H), 4.19 (dd, J=9.2, 10.4 Hz, 1H), 4.12-3.52 (m, 2H), 3.42-2.80 (m, 8H), 2.46-1.77 (m, 7H).

### Example 8

### 2-((S)-4-(7-(8-chloronaphthalen-1-yl)-2-(((2R,7aS)-2-fluorotetrahydro-1H-pyrrolizin-7a(5H)-yl)methoxy)-1,5-naphthyridin-4-yl)-1-(2-fluoroacryloyl)piperazin-2-yl)acetonitrile (1ah)

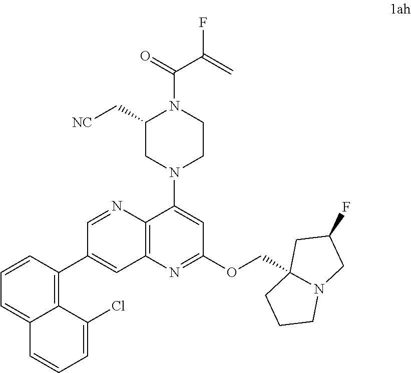 [SMILES: C=C(F)C(=O)N1CCN(c2cc(OC[C@@]34CCCN3C[C@H](F)C4)nc3cc(-c4cccc5cccc(Cl)c45)cnc23)C[C@@H]1CC#N]

### Example 8 (1ah) was Prepared as Shown Below in Scheme 9

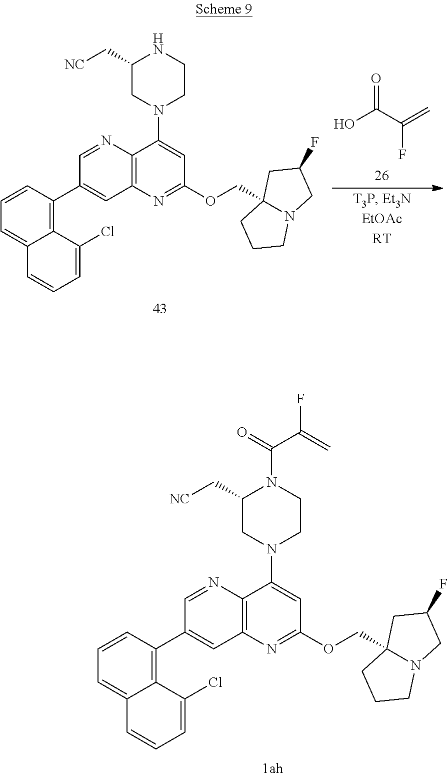

2-((S)-4-(7-(8-chloronaphthalen-1-yl)-2-(((2R,7aS)-2-fluorotetrahydro-1H-pyrrolizin-7a(5H)-yl)methoxy)-1,5-naphthyridin-4-yl)-1-(2-fluoroacryloyl)piperazin-2-yl)acetonitrile (1ah). 1-propanephosphonic anhydride solution (T₃P, 0.52 mL, 0.80 mmol, 50% in EtOAc) was added to a mixture of 2-((S)-4-(7-(8-chloronaphthalen-1-yl)-2-(((2R,7aS)-2-fluorotetrahydro-1H-pyrrolizin-7a(5H)-yl)methoxy)-1,5-naphthyridin-4-yl)piperazin-2-yl)acetonitrile (43; 83 mg, 0.15 mmol), 2-fluoroprop-2-enoic acid (26; CAS #430-99-9; 41 mg, 0.45 mmol) and Et₃N (0.12 mL, 0.80 mmol) in EtOAc (10 mL) under a N₂ atmosphere and the mixture stirred at RT forming a light pink reaction mixture. After 1 h, the light pink color had changed to a golden yellow. The mixture was stirred at RT, after 3 h, the reaction mixture was a light brown color. The mixture was diluted with EtOAc, washed with 5% aq. NaHCO₃ (3×), dried (MgSO₄), filtered, and concentrated in vacuo. The crude product was purified by silica gel column chromatography (eluting with a gradient of 0% to 10% MeOH in DCM) to afford 36 mg (39%) of 2-((S)-4-(7-(8-chloronaphthalen-1-yl)-2-(((2R,7aS)-2-fluorotetrahydro-1H-pyrrolizin-7a(5H)-yl)methoxy)-1,5-naphthyridin-4-yl)-1-(2-fluoroacryloyl)piperazin-2-yl)acetonitrile (1ah) as an off-white solid: HPLC-MS (ES⁺) m/z [M+H⁺]=643, 645; ¹H NMR (300 MHz, CDCl₃) δ 8.69 (t, J=2.2 Hz, 1H), 8.02 (dd, J=1.2, 1.9 Hz, 1H), 7.96 (dd, J=1.0, 8.1 Hz, 1H), 7.88 (dd, J=1.0, 8.1 Hz, 1H), 7.59-7.50 (m, 2H), 7.47-7.38 (m, 2H), 6.42 (d, J=3.7 Hz, 1H), 5.41 (br d, J=47.5 Hz, 1H), 5.29 (br d, J=53.2 Hz, 1H), 5.25 (dd, J=3.8, 17.0 Hz, 1H), 5.02 (br s, 1H), 4.87 (br s, 1H), 4.35 (dd, J=7.4, 10.6 Hz, 1H), 4.19 (dd, J=9.2, 10.3 Hz, 1H), 3.82 (br d, J=10.2 Hz, 1H), 3.54 (br s, 1H), 3.39-2.88 (m, 7H), 2.28-1.78 (m, 8H).

### Nucleotide Exchange Assays

The biological activity of the Examples was determined in a KRAS

G12D/SOS1 Nucleotide Exchange Assay that was performed by Reaction Biology Corporation (RBC), 1 Great Valley Parkway, Suite 2 Malvern, PA 19355, USA. The assay evaluates the SOS1-mediated Bodipy-GDP to GTP exchange observed with KRAS G12C and KRAS G12D.

The compounds were tested in 10 concentration IC₅₀ mode with 3-fold serial dilution at a starting concentration of 5 μM for ARS1620 and 10 UM for Examples 1-4, MRTX849 and MRTX1133. The compound pre-incubation time was 30 min at RT and the curve fits were performed when the activities at the highest concentration of compounds were less than 65%.

Reaction Buffer: 40 mM HEPES 7.4, 10 mM MgCl₂, 1 mM DTT 0.002% Triton X100, 0.5% DMSO.

Enzyme: SOS1 (RBC cat #MSC-11-502). Recombinant human SOS1 (Genbank accession #NM_033360.3; aa 564-1049, expressed in E. Coli with C-terminal StrepII).

KRAS G12C and KRAS G12D: Either recombinant human KRAS G12C or KRAS G12D (aa 2-169, expressed in E. coli with N-terminal TEV cleavable his-tag) was pre-loaded with a 5-fold excess of Bodipy™-GDP and the excess Bodipy-TMGDP was removed from loaded protein using a spin desalting column.

Final concentrations: KRAS-Bodipy™-GDP was 0.125 μM; SOS1 was 750 nM; and GTP was 25 μM.

### Reaction Procedure:

1. Deliver 10 μL of 1.5× KRAS solution in freshly prepared reaction buffer to reaction wells.
2. Deliver compounds in 100% DMSO into buffer using acoustic technology (Echo550; nanoliter range).
3. Incubate compounds with KRAS for 30 minutes at room temperature.
4. Prepare 3× (SOS1+GTP) solution in reaction buffer.
5. Deliver 5 μL of SOS1+GTP solution into reactions wells (deliver GTP only to column 1 for no SOS1 control).
6. Monitor reaction progress via decrease in fluorescence signal for 30 minutes at RT using a PHERAstar (BMG Labtech plate reader (Ex/Em=485/520).

Data Analysis: The fluorescence data was normalized using the equation below and fitted to “one phase exponential decay” equation using GraphPad prism software. The plateau was fixed to zero (use for non-covalent inhibitors) and rate ×1000 was used to calculate the IC₅₀ values.

$$Ynorm = 1 - \frac{( Yraw - Ao )}{( M - Ao )}$$

where Yraw is defined as fluorescence at time t, Ao is the average initial fluorescence with no SOS1, and Mis the minimum fluorescence at the end of the reaction at the maximum SOS1.

The background subtracted signals (no SOS1 protein wells were used as background) were converted to % activity relative to DMSO controls. Data was analyzed using GraphPad Prism 4 with “sigmoidal dose-response (variable slope)”; 4 parameters with Hill Slope. The constraints were bottom (constant equal to 0) and top (must be less than 120).

### Results:

KRAS G12C Activity

| Example | IC₅₀ (nM)* |
|---|---|
| ARS-1620 | 100 ± 17 (n = 8) |
| MRTX-849 | 28 ± 3 (n = 8) |
| Example 1 | 38 |
| (1b) | |
| Example 2 | 130 |
| (1q) | |
| Example 5 | 38 |
| (1ae) | |
| Example 6 | 59 |
| (1af) | |
| Example 7 | 31 |
| (1ag) | |
| Example 8 | 65 |
| (1ah) | |

*IC₅₀ values were calculated using the dRFU analysis method for covalent inhibitors with Bodipy-GDP/KRAS G12C as the substrate with 0.5% DMSO in the reaction. ARS-1620 and MRTX-849 are KRAS G12C reference standards.

KRAS G12D Activity

| Compound | IC₅₀ (nM)* |
|---|---|
| MRTX1133 | 11 ± 1 (n = 2) |
| Example 3 | 1,500 |
| (Compound 2aw) | |
| Example 4 | 1,000 |
| (Compound 2a) | |

*IC₅₀ values as analyzed by the rate constant method (plateau = 0) for reversible inhibitors with Bodipy-GDP/KRAS G12D as the substrate with 0.5% DMSO in the reaction. MRTX1133 is a KRAS G12D reference standard.

### KRAS G12C Cellular Assay

KRAS G12C cellular activity was determined in a target engagement cellular assay (NanoBRET™) in transiently transfected HEK293 cells by Reaction Biology Corporation (RBC), 1 Great Valley Parkway, Suite 2 Malvern, PA 19355, USA. HEK293 were cultivated to 70-80% confluence prior to the assay followed by trypsinizing and collection of the cells. BI-2852 was used as the KRAS G12C reference compound. Each test compound solution was delivered from a compound source plate to the wells of 384-well white non-binding surface plate by an Echo 550 prior to the assay.

A 10 μg/mL solution of DNA in Opti-MEM was prepared without serum that consisted of 1 μg LgBiT®-KRAS (G12C)-NanoLuc fusion vector, 1 μg SmBIT®-KRAS (G12C)-NanoLuc fusion vector and 8 μg transfection carrier DNA. This mixture was subsequently treated with 30 μL of FuGENE HD Transfection Reagent into each milliliter of DNA mixture to form a lipid: DNA complex. The resulting mixture was then gently mixed by inversion and incubated at ambient temperature for 20 minutes to allow complexes to form. A mixture of 1 part of lipid: DNA complex with 20 parts of suspended HEK293 cells was added to a sterile conical tube and mixed gently by inversion. The cells+lipid: DNA complex mixture was then added to a sterile tissue culture dish and incubated for 24 hours. The medium was removed from the dish via aspiration followed by trypsinizing and allowing the cells to dissociate from the tissue culture dish. The trypsin was subsequently neutralized by using medium containing serum and centrifugation at 200×g for 5 minutes to pellet the cells in the conical tube. The cell density was adjusted to 2×10⁵ cells/mL in Opti-MEM without phenol red. One part of Complete 20× NanoBRET™ RAS Tracer Reagent was dispensed to 20 parts of cells in the conical tube and mixed gently by inversion. The resulting cell suspension was dispensed into a white, 384-well NBS plate containing the test compounds (starting at 10 μM, 10-dose with 3-fold dilution) at 37° C., 5% CO₂ for 2 hours. The final concentration for RAS tracer K₂ was 1 μM. The NBS plate was removed from the incubator and allowed to equilibrate to room temperature for 15 minutes.

Freshly prepared substrate solution (3×) in the assay medium was added to each well of the 384-well NBS plate and incubated for 3 minutes at room temperature. The donor emission wavelength (460 nm) and acceptor emission wavelength (600 nm) were measured using an Envision 2104 plate reader. The raw BRET ratio values were generated by dividing the acceptor emission value (600 nm) by the donor emission value (460 nm) for each sample. In order to correct for the background, the BRET ratio in the absence of tracer (average of no-tracer control samples) was subtracted from the BRET ratio of each sample. The BRET ratio was calculated using the following equation: BRET Ratio= [(Acceptor sample÷Donor sample)−(Acceptor no-tracer control-Donor no-tracer control)]. The normalized BRET response (%) was calculated by the following equation: (BRET ratio of test compound/BRET ratio of DMSO control)*100%. The IC₅₀ curves were plotted and IC₅₀ values were calculated with GraphPad Prism 4 based on a sigmoidal dose-response equation.

### Results:

KRAS G12C Activity in Transiently Transfected HEK293 Cells*

| Compound | IC₅₀ (nM) |
|---|---|
| BI-2852 | 320 |
| Example 1 | 4.4 |
| (Compound 1b) | |
| Example 5 | <0.5 |
| (1ae) | |
| Example 6 | 334 |
| (1af) | |

*NanoBRET ™ target engagement cellular assay (KRAS G12C). BI-2852 is a KRAS G12C reference standard.

### CellTiter-Glo Viability Assay Protocol

### Materials:

The reference compound staurosporine was purchased from Sigma-Aldrich (Saint Louis, MI). CellTiter-Glo® 2.0 Luminescent cell viability assay reagent was purchased from Promega (Madison, WI). MIA PaCa-2 cell line was purchased from American Type Culture Collection (Manassas, VA). MIA PaCa-2 cells were cultured in DMEM containing 10% FBS, 2.5% horse serum, 100 μg/ml of penicillin and 100 μg/ml of streptomycin. Cultures were maintained at 37° C. in a humidified atmosphere of 5% CO₂ and 95% air.

### Procedure:

1. Test compounds and reference compound, staurosporine, were diluted in DMSO solution with 10-dose and 3-fold dilution in a source plate starting at 10 mM.
2. 25 nL of test compounds or 25 nL of staurosporine was delivered from the source plate to each well of the 384-well cell culture plate by Echo 550.
3. 25 μL of culture media containing 2000 cells was added to each of the wells of the cell culture plate in duplicate.
4. The cells were incubated with the compounds at 37° C., 5% CO₂ for 72 hours.
5. 25 μL of CellTiter-Glo 2.0 reagent was added to each well.
6. The contents were mixed on an orbital shaker for 2 min and incubated at room temperature for 15 min to stabilize luminescent signal.
7. Luminescence was recorded by Envision 2104 Multilabel Reader (PerkinElmer, Santa Clara, CA). The number of viable cells in culture was determined based on quantitation of the ATP present in each culture well.
8. The IC50 curves were plotted and IC50 values were calculated using the GraphPad Prism 4 program based on a sigmoidal dose-response equation

### Results:

CTG Assay IC₅₀ Values (M)

| Compound/Cell Line | MIA PaCa-2 |
|---|---|
| MRTX-849 | 1.52E−06 |
| Example 5 | 2.88E−06 |
| 1ae | |
| Staurosporine | 2.49E−07 |

PK Profile in male CD1 mice:

| Analyte | 1ae | |
|---|---|---|
| Group | 3 mg/kg, i.v., single | 30 mg/kg, p.o., single |
| | dose | dose |
| | (20% DMSO/30% | (1% Tween 80/2% |
| | PEG400/ 50% ddH2O) | HPMC/ 97% H2O) |
| Matrix | Mean Plasma | Mean Plasma |
| Dosage (mg/kg) | 3.00 | 30.0 |
| Rsq_adj | 0.955 | 0.993 |
| No. points used for T_{1/2} | 6.00 | 5.00 |
| C₀ (ng/mL) | 800 | — |
| Cₘₐₓ (ng/mL) | — | 249 |
| Tₘₐₓ (h) | — | 1.00 |
| T_{1/2} (h) | 1.42 | 2.85 |
| Vdss (L/kg) | 7.70 | — |
| Cl(mL/min/kg) | 72.4 | — |
| Tₗₐₛₜ (h) | 8.00 | 24.0 |
| AUC₀₋ₗₐₛₜ (ng · h/mL) | 673 | 1495 |
| AUC_{0-inf} (ng · h/mL) | 691 | 1499 |
| MRT₀₋ₗₐₛₜ (h) | 1.56 | 4.72 |
| MRT_{0-inf} (h) | 1.77 | 4.79 |
| AUC_{Extra} (%) | 2.52 | 0.315 |
| AUMC_{Extra} (%) | 14.3 | 1.85 |
| Bioavailability (%) | | 21.7 |

What is claimed is:

1. A compound of Formula 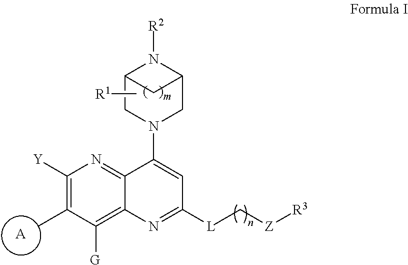
or a pharmaceutically acceptable salt, solvate, or prodrug thereof, wherein
A is chosen from aryl or heteroaryl optionally substituted with one or more of hydrogen, halogen, hydroxy, —C₁₋₆ alkyl, —C₂₋₆ alkenyl, —C₂₋₆ alkynyl, —C₀₋₃ alkyl(C₃₋₆ cycloalkyl), —C₁₋₆ alkyl(halo), —C₁₋₆ alkyl(OH), —O(C₁₋₄ alkyl), —C₁₋₃ alkyl(C₁₋₄ alkoxy), —CN, —CO₂R⁴, —CO₂N(R⁴)₂, —NO₂, —N(R⁴)₂, —P(O)(R⁵)₂, —SR₄, —S(O)R⁴, —SO₂R⁴ or a 5-6 membered heterocyclic ring;
Y and G may be the same or different and chosen from hydrogen, halogen, C₁₋₄ alkyl, C₁₋₄ perdeuteroalkyl, —(C₀₋₂ alkyl)alkenyl, —(C₀₋₂ alkyl)alkynyl, —(C₀₋₂ alkyl)cycloalkyl, —C₁₋₄ haloalkyl, —O(C₁₋₄ alkyl), —S(C₁₋₄ alkyl), —(C₀₋₂ alkyl) cyano, —O(C₁₋₄ haloalkyl) or —S(C₁₋₄ haloalkyl);
L is a bond, O, S, or NR⁴;
m is 0-2;
n is 0-2;
Z is C(R⁴)₂ or a cyclic compound chosen from C₃₋₇ cycloalkyl, a saturated or partially unsaturated 4- to 7-membered nitrogen-containing ring, a saturated or partially unsaturated 7- to 10-membered nitrogen-containing bridged bicyclic ring;
R¹ is chosen from hydrogen, hydroxy, halogen, —C₁₋₃ alkyl, —C₁₋₃ alkyl (OH), —C₁₋₃ alkyl(halo), —C₁₋₃ alkyl(C₁₋₃ alkoxy), —C₁₋₃ alkyl(CN) or —C₁₋₃ alkyl(P(O)R⁵₂);
R² is chosen from hydrogen, —C(O)CH═CH, —C(O)CF═CH or —C(O)CCl═CH with the proviso that when R² is hydrogen, then m is either 1 or 2;
R³ is chosen from hydrogen, halogen, hydroxy, —C₁₋₄ alkyl, —C₂₋₄ alkenyl, —C₂₋₄ alkynyl, —C₀₋₃ alkyl(C₃₋₆ cycloalkyl), —C₁₋₄ alkyl(halo), —C₁₋₄ alkyl(OH), —O(C₁₋₄ alkyl), —C₁₋₃ alkyl(C₁₋₃ alkoxy), —CN, —CO₂R⁴, —CO₂N(R⁴)₂, —NO₂, —N(R⁴)₂, —PO(R⁵)₂, —SR₄, —S(O)R⁴, —SO₂R⁴, or —(C₀₋₃ alkyl)R⁶;
R⁴ is chosen from is chosen from hydrogen, C₁₋₄ alkyl, aryl or heteroaryl;
R⁵ is chosen from hydrogen, hydroxy, C₁₋₄ alkyl, aryl, heteroaryl, C₁₋₄ alkoxy, aryloxy or heteroaryloxy;
R⁶ is chosen from N(R⁴)₂ or a 4- to 7-membered saturated or unsaturated heterocyclic ring containing one or more heteroatoms selected from the group N, O and S.

2. The compound of claim 1 wherein the compound of Formula I is selected from compounds 1a-1ah and 2a-2aw, including a pharmaceutically acceptable salt, solvate, or prodrug thereof:

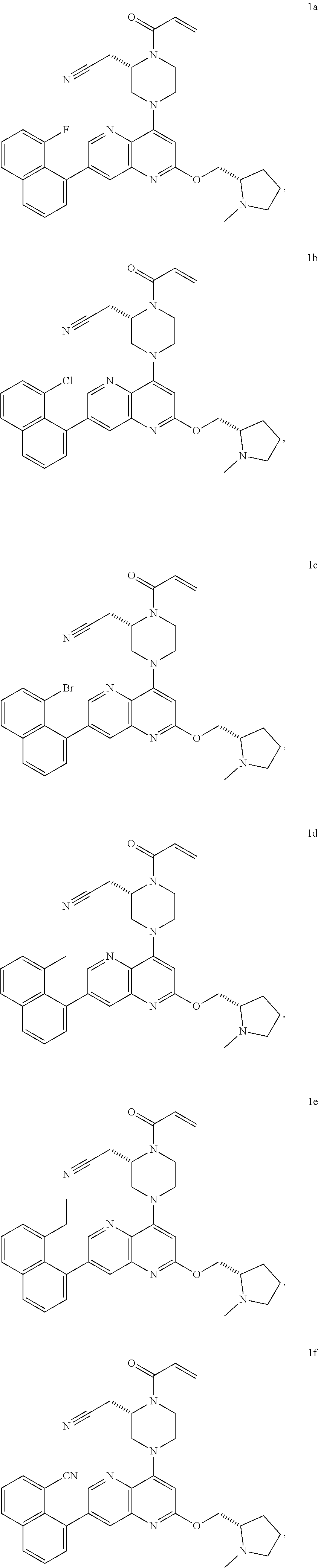 [SMILES: C=CC(=O)N1CCN(c2cc(OC[C@@H]3CCCN3C)nc3cc(-c4cccc5cccc(F)c45)cnc23)C[C@@H]1CC#N ; C=CC(=O)N1CCN(c2cc(OC[C@@H]3CCCN3C)nc3cc(-c4cccc5cccc(Cl)c45)cnc23)C[C@@H]1CC#N ; C=CC(=O)N1CCN(c2cc(OC[C@@H]3CCCN3C)nc3cc(-c4cccc5cccc(Br)c45)cnc23)C[C@@H]1CC#N ; C=CC(=O)N1CCN(c2cc(OC[C@@H]3CCCN3C)nc3cc(-c4cccc5cccc(C)c45)cnc23)C[C@@H]1CC#N ; C=CC(=O)N1CCN(c2cc(OC[C@@H]3CCCN3C)nc3cc(-c4cccc5cccc(CC)c45)cnc23)C[C@@H]1CC#N ; C=CC(=O)N1CCN(c2cc(OC[C@@H]3CCCN3C)nc3cc(-c4cccc5cccc(C#N)c45)cnc23)C[C@@H]1CC#N]

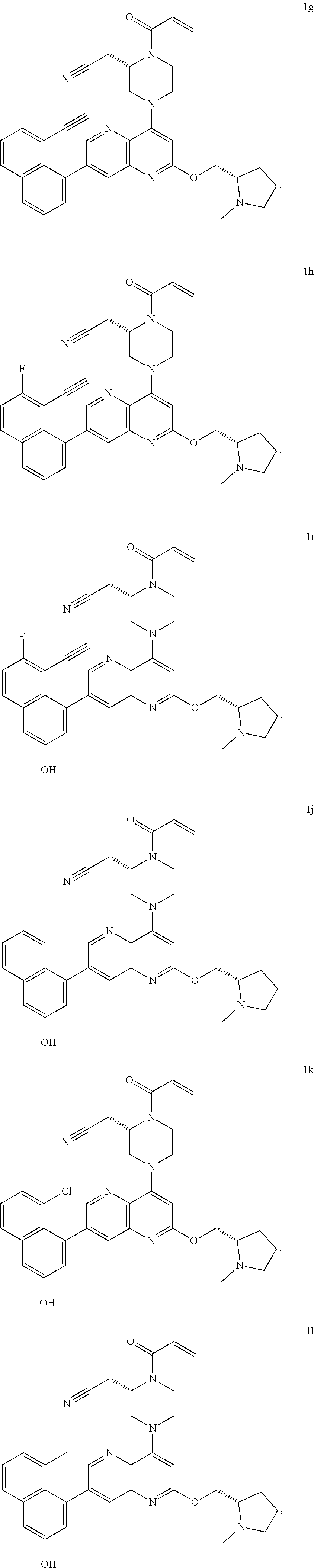

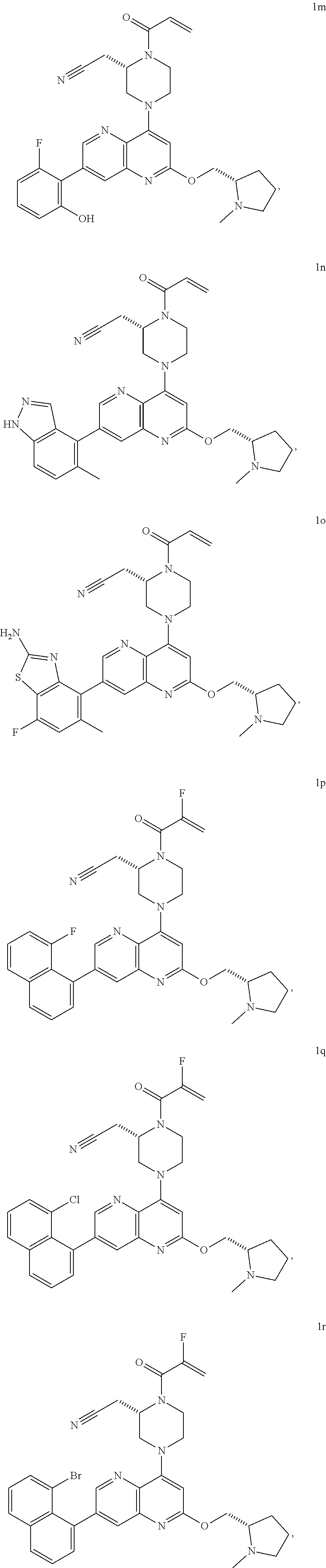

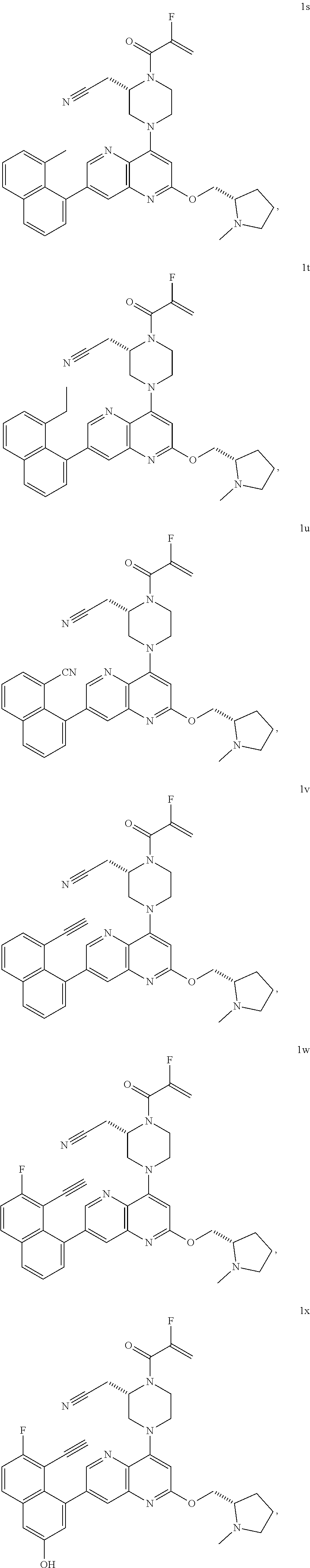

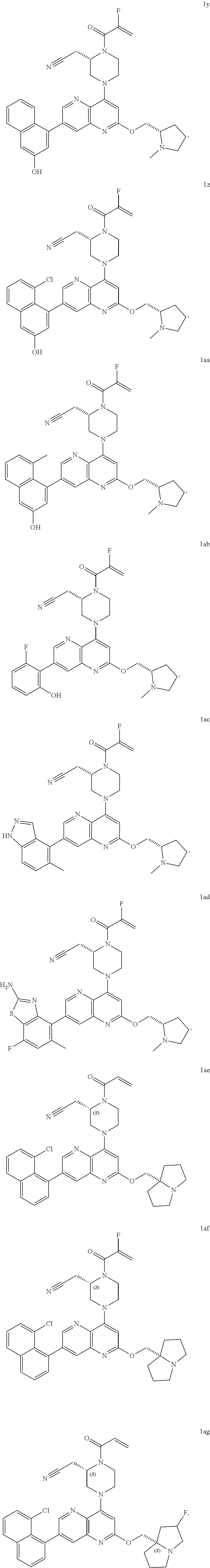

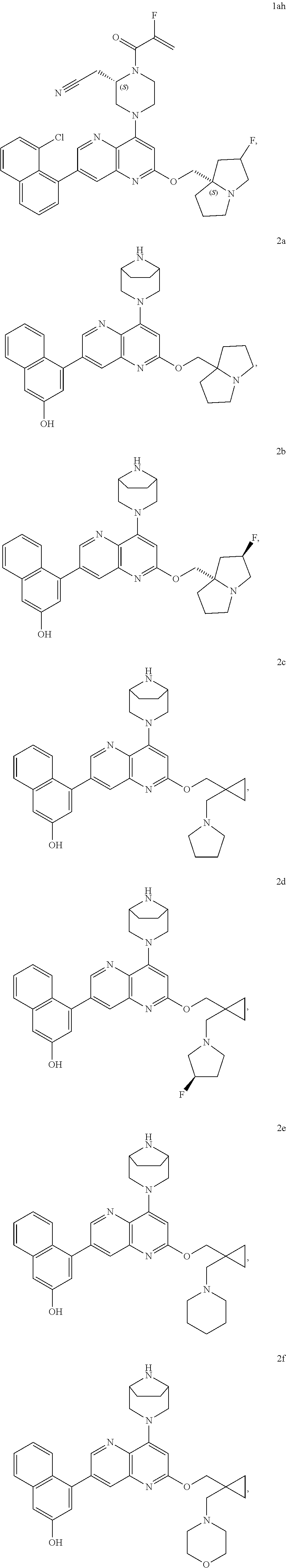

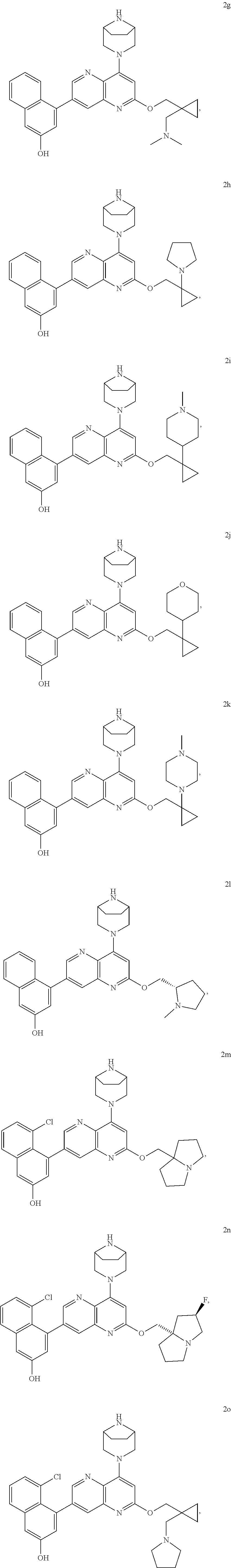

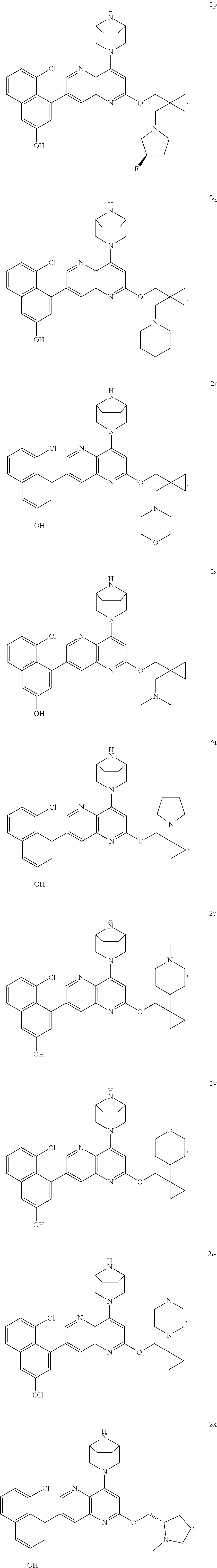

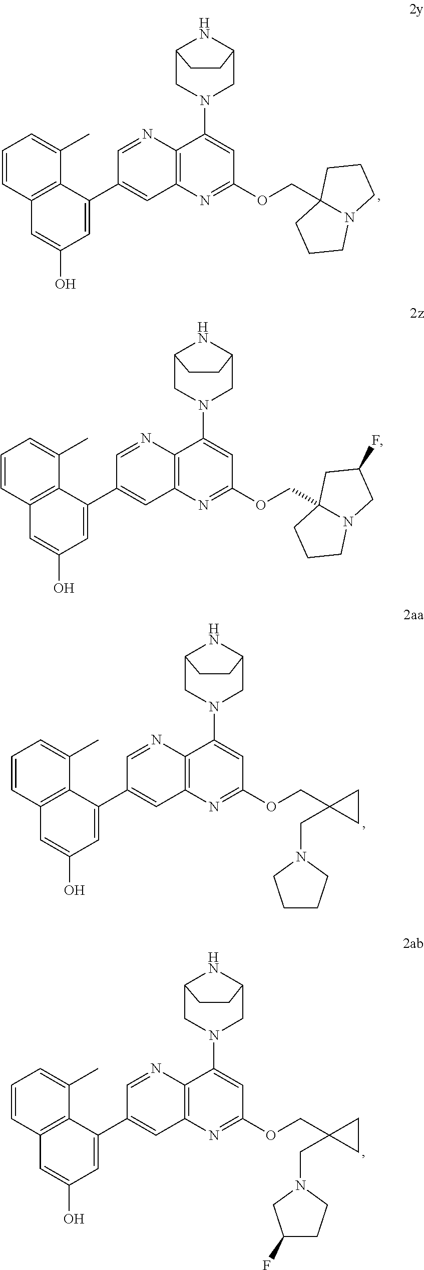 [SMILES: Cc1cccc2cc(O)cc(-c3cnc4c(N5CC6CCC(C5)N6)cc(OCC56CCCN5CCC6)nc4c3)c12 ; Cc1cccc2cc(O)cc(-c3cnc4c(N5CC6CCC(C5)N6)cc(OC[C@@]56CCCN5C[C@H](F)C6)nc4c3)c12 ; Cc1cccc2cc(O)cc(-c3cnc4c(N5CC6CCC(C5)N6)cc(OCC5(CN6CCCC6)CC5)nc4c3)c12 ; Cc1cccc2cc(O)cc(-c3cnc4c(N5CC6CCC(C5)N6)cc(OCC5(CN6CC[C@@H](F)C6)CC5)nc4c3)c12]

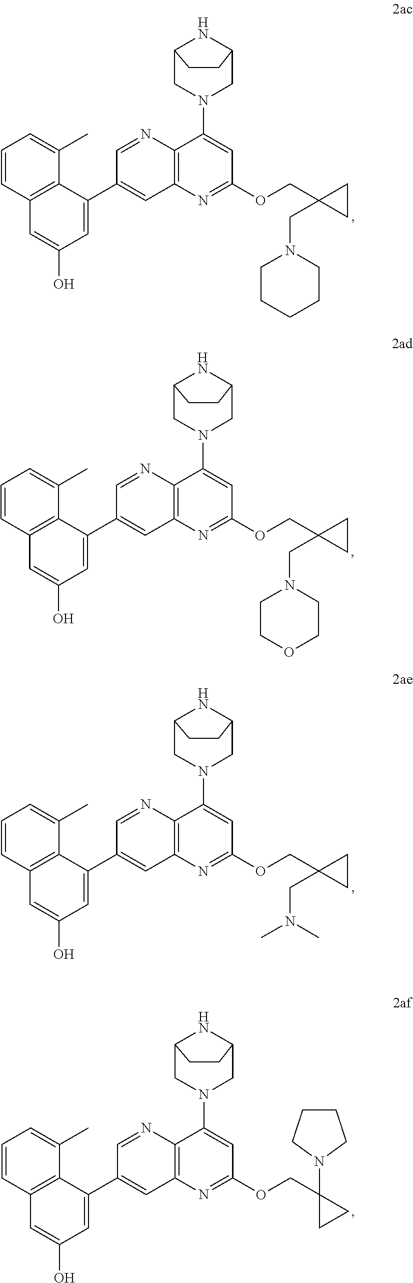 [SMILES: Cc1cccc2cc(O)cc(-c3cnc4c(N5CC6CCC(C5)N6)cc(OCC5(CN6CCCCC6)CC5)nc4c3)c12 ; Cc1cccc2cc(O)cc(-c3cnc4c(N5CC6CCC(C5)N6)cc(OCC5(CN6CCOCC6)CC5)nc4c3)c12 ; Cc1cccc2cc(O)cc(-c3cnc4c(N5CC6CCC(C5)N6)cc(OCC5(CN(C)C)CC5)nc4c3)c12 ; Cc1cccc2cc(O)cc(-c3cnc4c(N5CC6CCC(C5)N6)cc(OCC5(N6CCCC6)CC5)nc4c3)c12]

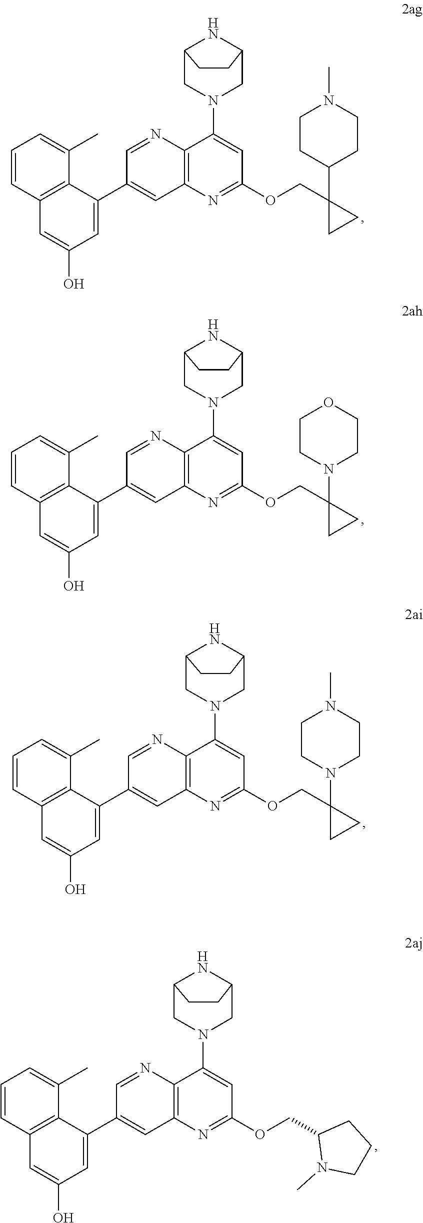 [SMILES: Cc1cccc2cc(O)cc(-c3cnc4c(N5CC6CCC(C5)N6)cc(OCC5(C6CCN(C)CC6)CC5)nc4c3)c12 ; Cc1cccc2cc(O)cc(-c3cnc4c(N5CC6CCC(C5)N6)cc(OCC5(N6CCOCC6)CC5)nc4c3)c12 ; Cc1cccc2cc(O)cc(-c3cnc4c(N5CC6CCC(C5)N6)cc(OCC5(N6CCN(C)CC6)CC5)nc4c3)c12 ; Cc1cccc2cc(O)cc(-c3cnc4c(N5CC6CCC(C5)N6)cc(OC[C@@H]5CCCN5C)nc4c3)c12]

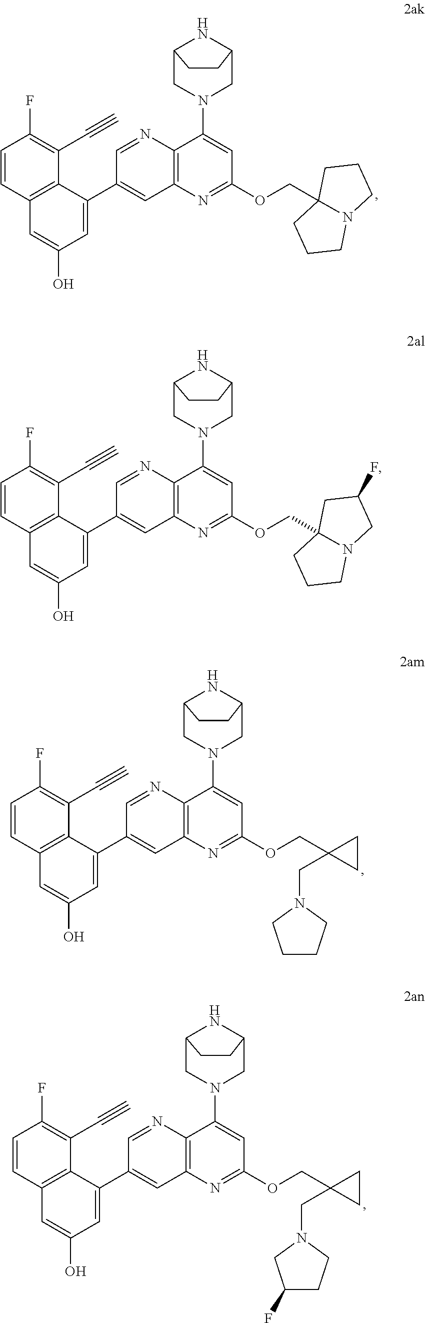 [SMILES: C#Cc1c(F)ccc2cc(O)cc(-c3cnc4c(N5CC6CCC(C5)N6)cc(OCC56CCCN5CCC6)nc4c3)c12 ; C#Cc1c(F)ccc2cc(O)cc(-c3cnc4c(N5CC6CCC(C5)N6)cc(OC[C@@]56CCCN5C[C@H](F)C6)nc4c3)c12 ; C#Cc1c(F)ccc2cc(O)cc(-c3cnc4c(N5CC6CCC(C5)N6)cc(OCC5(CN6CCCC6)CC5)nc4c3)c12 ; C#Cc1c(F)ccc2cc(O)cc(-c3cnc4c(N5CC6CCC(C5)N6)cc(OCC5(CN6CC[C@@H](F)C6)CC5)nc4c3)c12]

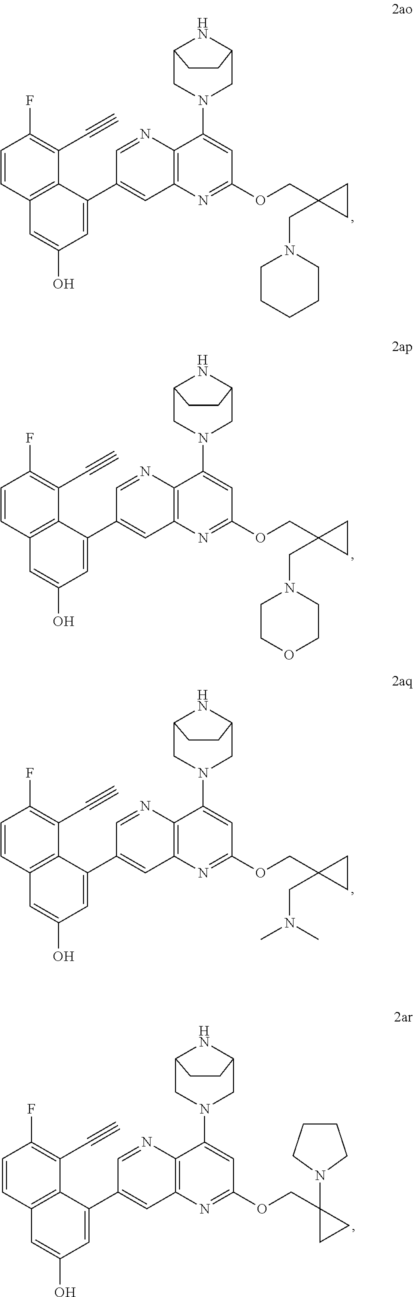 [SMILES: C#Cc1c(F)ccc2cc(O)cc(-c3cnc4c(N5CC6CCC(C5)N6)cc(OCC5(CN6CCCCC6)CC5)nc4c3)c12 ; C#Cc1c(F)ccc2cc(O)cc(-c3cnc4c(N5CC6CCC(C5)N6)cc(OCC5(CN6CCOCC6)CC5)nc4c3)c12 ; C#Cc1c(F)ccc2cc(O)cc(-c3cnc4c(N5CC6CCC(C5)N6)cc(OCC5(CN(C)C)CC5)nc4c3)c12 ; C#Cc1c(F)ccc2cc(O)cc(-c3cnc4c(N5CC6CCC(C5)N6)cc(OCC5(N6CCCC6)CC5)nc4c3)c12]

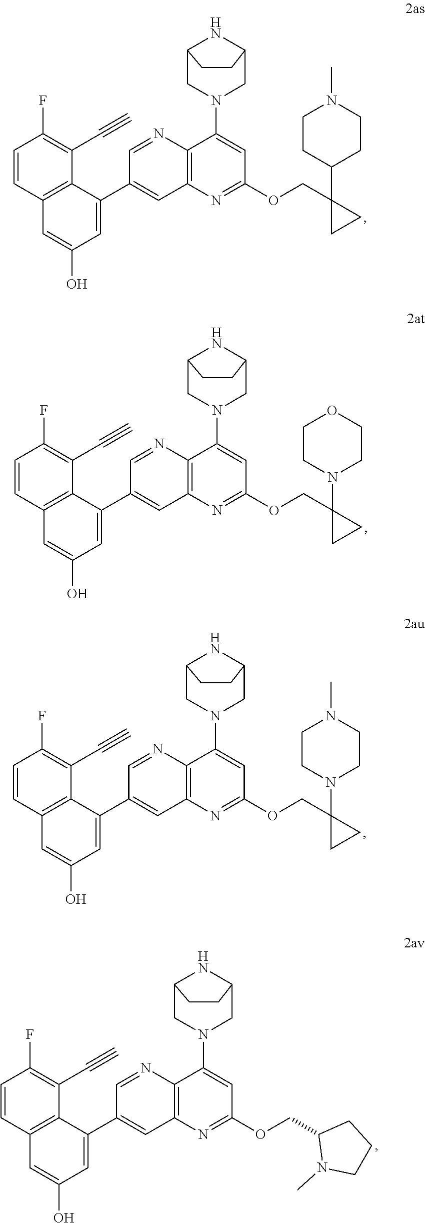 [SMILES: C#Cc1c(F)ccc2cc(O)cc(-c3cnc4c(N5CC6CCC(C5)N6)cc(OCC5(C6CCN(C)CC6)CC5)nc4c3)c12 ; C#Cc1c(F)ccc2cc(O)cc(-c3cnc4c(N5CC6CCC(C5)N6)cc(OCC5(N6CCOCC6)CC5)nc4c3)c12 ; C#Cc1c(F)ccc2cc(O)cc(-c3cnc4c(N5CC6CCC(C5)N6)cc(OCC5(N6CCN(C)CC6)CC5)nc4c3)c12 ; C#Cc1c(F)ccc2cc(O)cc(-c3cnc4c(N5CC6CCC(C5)N6)cc(OC[C@@H]5CCCN5C)nc4c3)c12]

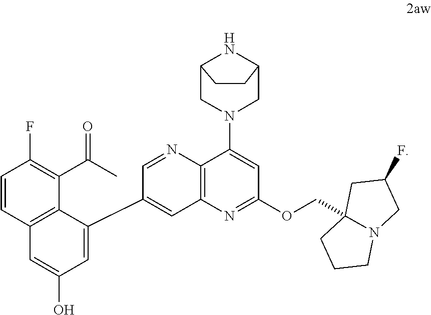 [SMILES: CC(=O)c1c(F)ccc2cc(O)cc(-c3cnc4c(N5CC6CCC(C5)N6)cc(OC[C@@]56CCCN5C[C@H](F)C6)nc4c3)c12]

3. A pharmaceutical composition comprising the compound of any one of claims 1 to 2, or a salt, solvate, or prodrug thereof, together with a pharmaceutically acceptable carrier.

4. A method of treating a disease, disorder, or medical condition in a patient, comprising the step of providing to a patient in need thereof a therapeutic agent, wherein the therapeutic agent is a compound of any one of claims 1 to 3, or a salt, solvate, or prodrug thereof.

5. The method of treating a disease, disorder, or medical condition of claim 4, wherein said disease includes various cancers.

6. The method of treating a disease, disorder, or medical condition of claim 5, wherein said disease, disorder, or medical condition is mediated through KRAS.

7. The method of treating a disease, disorder, or medical condition of claim 6, wherein said disease, disorder, or medical condition is mediated through the KRAS mutants G12C or G12D.

8. The method of treating a disease, disorder, or medical condition of claim 5, wherein the cancer is selected from glioma (glioblastoma), acute myelogenous leukemia, acute myeloid leukemia, myelodysplastic/myeloproliferative neoplasms, sarcoma, chronic myelomonocytic leukemia, non-Hodgkin lymphoma, astrocytoma, melanoma, non-small cell lung cancer, small cell lung cancer, cholangiocarcinomas, chondrosarcoma, colon cancer, colorectal cancer, rectal cancer or pancreatic cancer.

9. The method of any one of claims 4 to 8, further comprising administering to the patient in need thereof at least one additional therapeutic agent.

10. The method of claim 9 wherein the additional therapeutic agent is selected from doxorubicin, paclitaxel, docetaxel, cisplatin, camptothecin, temozolomide, avastin, Herceptin, Erbitux, EGFR inhibitors, osimertinib, rezivertinib, CDK 4/6 inhibitors, abemaciclib, palbociclib, ribociclib, c-MET inhibitors, capmatinib, volitinib, ALK inhibitors, crizotinib, alectinib, ceritinib, brigatinib, entrectinib, lorlatinib, PD-1 antagonists, PD-L1 antagonists, ipilimumab, embrolizumab, or nivolumab.

11. The compound of claim 2 wherein the compound of Formula I is selected from compounds 1ae-1ah, 1b, 1d, 1i, 1q, 1s, 1x, 2z, 2al, 2an, 2ap and 2aq or a salt, solvate, or prodrug thereof:

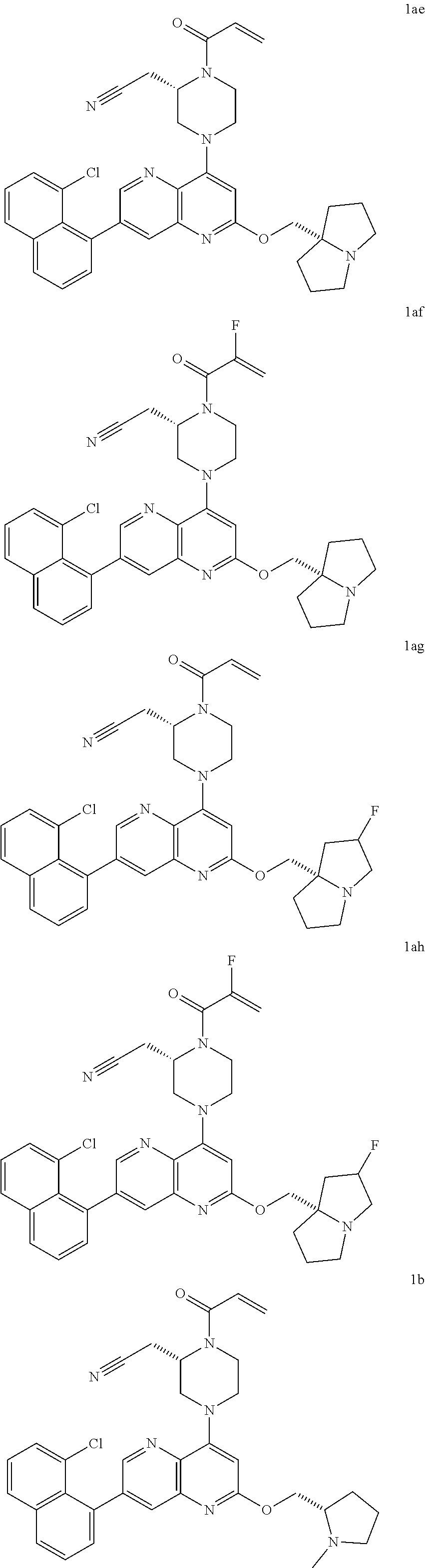

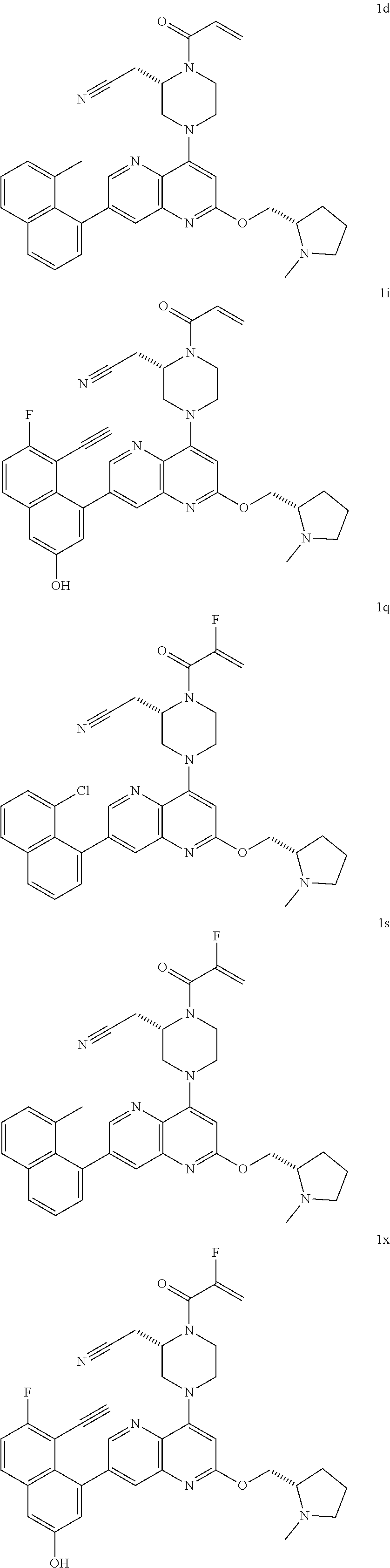

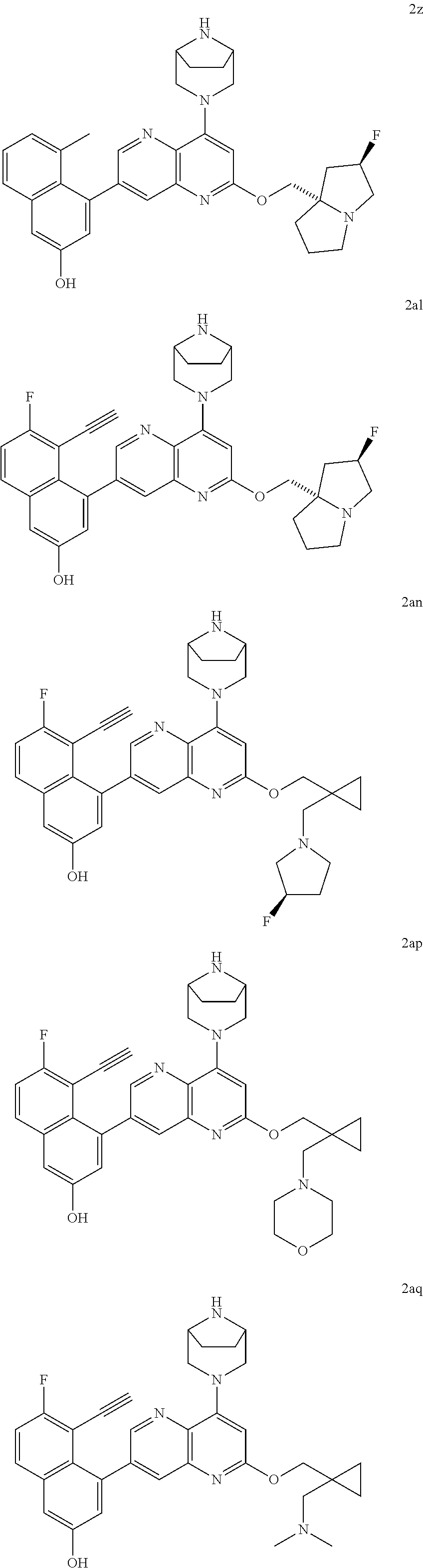

12. The pharmaceutical composition of claim 3, comprising compound either 1ae-1ah, 1b, 1q or 2z or a salt, solvate, or prodrug thereof together with a pharmaceutically acceptable carrier.

13. The method of claim 4, comprising the step of providing to a patient in need thereof a pharmaceutical composition of claim 12.

14. The method of claim 13, wherein said diseases include various cancers.

15. The method of claim 14, wherein said disease, disorder, or medical condition is mediated through KRAS.

16. The method of claim 15, wherein said disease, disorder, or medical condition is mediated through KRAS mutants G12C or G12D.

17. The method of claim 14, wherein the cancer is selected from glioma (glioblastoma), acute myelogenous leukemia, acute myeloid leukemia, myelodysplastic/myeloproliferative neoplasms, sarcoma, chronic myelomonocytic leukemia, non-Hodgkin lymphoma, astrocytoma, melanoma, non-small cell lung cancer, small cell lung cancer, cholangiocarcinomas, chondrosarcoma, colon cancer, colorectal cancer, rectal cancer or pancreatic cancer.
